(19) United States
(12) Patent Application Publication
(10) Pub. No.: US 2022/0195029 A1
(43) Pub. Date: Jun. 23, 2022

(54) METHOD OF PRODUCING A RECOMBINANT PROTEIN

(71) Applicant: Coherus Biosciences, Inc., Redwood City, CA (US)

(72) Inventors: Robin Modi, San Jose, CA (US); James Russell Grove, Redwood City, CA (US)

(73) Assignee: Coherus Biosciences, Inc., Redwood City, CA (US)

(21) Appl. No.: 17/608,204

(22) PCT Filed: May 1, 2020
(86) PCT No.: PCT/US2020/031088

§ 371 Date: Nov. 2, 2021

Related U.S. Application Data
(60) Provisional application No. 62/843,270, filed on May 3, 2019.

(51) Int. Cl. C07K 16/24; C12N 5/071

(52) U.S. Cl. CPC C07K 16/241; C12N 5/0682; C12N 2500/34; C12N 2500/60; C12N 2510/02

(57) ABSTRACT

Provided herein are methods of producing a recombinant protein that include fed-batch culturing a mammalian cell.

### CROSS-REFERENCE TO RELATED APPLICATIONS

This application claims priority to U.S. Provisional Application Ser. No. 62/843,270, filed on May 3, 2019, the entire contents of which is incorporated herein by reference.

### TECHNICAL FIELD

This invention relates to methods of molecular biology, cell culture process development, and the manufacture of recombinant proteins.

### BACKGROUND

Mammalian cells containing a nucleic acid that encodes a recombinant protein are often used to produce therapeutically or commercially important proteins.

### SUMMARY

The present invention is based, at least in part, on the discovery that culturing methods that include the use of three different chemically-defined animal component-free liquid culture media result in improved culturing characteristics such as product titer.

Additionally, the culturing methods produce a recombinant protein with desired characteristics (e.g. N-glycosylation profile). Thus provided herein are methods of producing a recombinant protein that include: (a) providing a cell culture comprising a mammalian cell (e.g., a CHO cell) in a liquid culture medium (e.g., a chemically-defined, animal component-free liquid culture medium), where the CHO cell includes a nucleic acid encoding a recombinant protein; (b) fed-batch culturing the cell culture of step (a) under conditions sufficient for the CHO cell to produce the recombinant protein, where: the fed-batch culturing includes adding a volume of a first feed culture medium (e.g., a chemically-defined, animal component-free liquid culture medium, e.g., about 0.8× to about 1.0× BalanCD™ CHO Feed 4) on about day 2 to about day 5 of the culture and adding a volume of a second feed culture medium (e.g., a chemically-defined, animal component-free liquid culture medium, e.g., about 0.9× to about 1.1× BalanCD™ CHO Feed 2) on about day 6 to about day 13; and (c) recovering the recombinant protein from the mammalian cell or the liquid culture medium. Also provided are recombinant proteins produced by these methods and methods of treating a subject in need thereof that include administering a therapeutically effective amount of a recombinant protein produced by any of the methods described herein to the subject.

Provided herein is a method of producing a recombinant protein, the method including (a) providing a cell culture comprising a CHO cell in a liquid culture medium, wherein the CHO cell comprises a nucleic acid encoding a recombinant protein, wherein the cell culture has a volume, (b) fed-batch culturing the cell culture of step (a) under conditions sufficient for the CHO cell to produce the recombinant protein, wherein the fed-batch culturing includes adding a volume of a first feed culture medium comprising 0.8× to 1.0× BalanCD™ CHO Feed 4 on about day 2 to about day 5 of the culture and adding a volume of a second feed culture medium comprising 0.9× to 1.1× BalanCD′ CHO Feed 2 on about day 6 to about day 13, and (c) recovering the recombinant protein from the CHO cell or the liquid culture medium.

Implementations can include one or more of the following features. One or more of the liquid culture medium, the first feed culture medium, and the second feed culture medium can further include a concentration of N-acetylglucosamine sufficient to maintain a concentration of N-acetylglucosamine in the cell culture of about 2 mM to about 8 mM relative to the volume of the cell culture in step (a). The fed-batch culturing can further include adding a volume of a supplement comprising a concentration of N-acetylglucosamine sufficient to maintain a concentration of N-acetylglucosamine in the cell culture of about 2 mM to about 8 mM. The volume of the first feed culture medium added on about day 2 to about day 5 can be about 4% to about 10% of the volume of the cell culture in step (a) per day. The volume of the first feed culture medium added on about day 2 to about day 5 can be about 5% of the volume of the cell culture in step (a) per day. The volume of the second feed culture medium added on about day 6 to about day 13 can be about 4% to about 10% of the volume of the cell culture in step (a) per day. The volume of the second feed culture medium added on about day 6 to about day 13 can be about 5% to about 7% of the volume of the cell culture in step (a) per day. The first feed culture medium can include about 0.8× BalanCD™ CHO Feed 4. The second feed culture medium can include about 1.0× BalanCD′ CHO Feed 2. The fed-batch culturing can further include adjusting the temperature of the culture on about day 7 to about day 8. The temperature of the culture can be adjusted from a first temperature of about 35-38° C. to a second temperature of about 28-34.9° C. The temperature of the culture can be adjusted from a first temperature of about 36.5° C. to a second temperature of about 34° C. The fed-batch culturing can further include maintaining the pH of the cell culture at about 6.7 to about 7.1. Upon the cell culture obtaining a pH of 6.9, the pH can be maintained at about 6.88 to about 6.92. The fed-batch culturing can further include maintaining the dO₂ of 40%. The fed-batch culturing can further include agitating the cell culture at about 10 RPM to about 500 RPM. The fed-batch culturing can further include agitating the cell culture at about 180 RPM to about 220 RPM. The fed-batch culturing can further include agitating the cell culture using an impeller tip speed of 0.4 m/s to about 4.0 m/s. The fed-batch culturing can further include agitating the cell culture using an impeller power consumption per volume of about 10 W/m³ to about 35 W/m³. The recovering in step (c) can occur on day 14. The cell culture can have a percent of cell viability, and the recovering in step (c) can occur when the percent of cell viability falls below a value selected from the group consisting of about 70%, about 60%, about 50%, about 40%, and about 30%. The CHO cell can be a DG44 cell. The first feed culture medium and the second feed culture medium can further include about 4 g/L glucose to about 6 g/L glucose. The first feed culture medium and the second feed culture medium can include about 5 g/L glucose. The recombinant protein can be a fusion protein, antibody, or antibody fragment. The method can further include generating the cell culture of step (a) including inoculating the liquid culture medium with a population of the CHO cells. The population of the CHO cells can have not been previously cultured in the liquid culture medium. The liquid culture medium can be HyClone™ ActiPro™. The liquid culture medium can be CD-C4. The method can further include purifying the recovered recombinant protein. The method can further include formulating the purified recombinant protein into a pharmaceutical composition.

Also provided herein is a recombinant protein produced by any of the methods described herein.

Also provided herein is a pharmaceutical composition produced by any of the methods described herein.

Also provided herein is a method of treating a subject in need thereof comprising administering to the subject a therapeutically effective amount of any of the recombinant proteins described herein or any of the pharmaceutical compositions described herein.

As used herein, the word “a” before a noun represents one or more of the particular noun. For example, the phrase “a mammalian cell” represents “one or more mammalian cells.”

The term “mammalian cell” means any cell from or derived from any mammal (e.g., a human, a hamster, a mouse, a green monkey, a rat, a pig, a cow, or a rabbit). In some embodiments, a mammalian cell can be an immortalized cell. In some embodiments, the mammalian cell is a differentiated cell. In some embodiments, the mammalian cell is an undifferentiated cell. In some embodiments, a mammalian cell can be a CHO cell (e.g., a DG44 cell). A variety of different commercially available CHO cells (including DG44 cells) are known in the art.

The term “day 0” means the time point at which a mammalian cell is seeded into the liquid culture medium.

The term “day 1” means a time period between day 0 and about 24 hours following the seeding of a mammalian cell into the liquid culture medium.

The term “day 2” means a time period of about 24 hours to about 48 hours following the seeding of a mammalian cell into the liquid culture medium.

The term “day 3” means a time period of about 48 hours to about 72 hours following the seeding of a mammalian cell into the liquid culture medium.

The term “day 4” means a time period of about 72 hours to about 96 hours following the seeding of a mammalian cell into the liquid culture medium. The term for each additional day (“day 5,” “day 6,” “day 7,” and so on) is meant a time period that ranges over an additional about 24-hour period from the end of the immediately preceding day.

The term “culturing” or “cell culturing” is meant the maintenance or growth of a mammalian cell under a controlled set of physical conditions.

The term “liquid culture medium” means a fluid that contains sufficient nutrients to allow a mammalian cell to grow in vitro. For example, a liquid culture medium can contain one or more of: amino acids (e.g., 20 amino acids), a purine (e.g., hypoxanthine), a pyrimidine (e.g., thymidine), choline, inositol, thiamine, folic acid, biotin, calcium, niacinamide, pyridoxine, riboflavin, thymidine, cyanocobalamin, pyruvate, lipoic acid, magnesium, glucose, sodium, potassium, iron sulfate, copper sulfate, zinc sulfate, and sodium bicarbonate. In some embodiments, a liquid culture medium can contain serum from a mammal. In some embodiments, a liquid culture medium does not contain serum or another extract from a mammal (a defined liquid culture medium). In some embodiments, a liquid culture medium can contain trace metals, a mammalian growth hormone, and/or a mammalian growth factor. Non-limiting examples of liquid culture medium are described herein. Additional examples of liquid culture medium are known in the art and are commercially available. A liquid culture medium can contain any density of mammalian cells. For example, as used herein, a first volume of the first culture medium removed from the container can be substantially free of mammalian cells.

The term “animal component free liquid culture medium” means a liquid culture medium that does not contain any components (e.g., proteins or serum) derived from a mammal.

The term “serum-free liquid culture medium” means a liquid culture medium that does not contain the serum of a mammal.

The term “chemically-defined liquid culture medium” means a liquid culture medium in which all of the chemical components are known. For example, a chemically-defined liquid culture medium does not contain fetal bovine serum, bovine serum albumin, or human serum albumin, as these preparations typically contain a complex mix of albumins and lipids.

The term “protein-free liquid culture medium” means a liquid culture medium that does not contain any protein (e.g., any detectable protein).

The term “agitation” means the movement of a container containing a liquid culture medium in order to increase the dissolved O₂ concentration in the liquid culture medium. Agitation can be performed using any art known method, e.g., an instrument that moves a vessel containing a cell culture in a circular or ellipsoidal motion, such as a rotary shaker. Alternatively or in addition, agitation can be performed by tilting the container or rolling a vessel containing a cell culture. In some embodiments, agitation of a cell culture can occur through the use of an impeller in a bioreactor containing the cell culture.

The term “recovering” means partially purifying or isolating (e.g., at least or about 5%, e.g., at least or about 10%, 15%, 20%, 25%, 30%, 40%, 45%, 50%, 55%, 60%, 65%, 70%, 75%, 80%, 85%, 90%, or at least or about 95% pure by weight) a recombinant protein from one or more other components present in the cell culture medium (e.g., mammalian cells or culture medium proteins) or one or more other components (e.g., DNA, RNA, or other proteins) present in a mammalian cell lysate. Non-limiting methods for recovering a protein from a liquid culture medium or from a mammalian cell lysate are described herein and others are known in the art.

The term “secreted protein” or “secreted recombinant protein” means a protein or a recombinant protein that originally contained at least one secretion signal sequence when it is translated within a mammalian cell, and through, at least in part, enzymatic cleavage of the secretion signal sequence in the mammalian cell, is released into the extracellular space (e.g., a liquid culture medium).

The term “fed-batch culture” or “fed-batch culturing” means the incremental or continuous addition of one or more feed liquid culture media to an initial cell culture without substantial or significant removal of liquid culture medium from the cell culture.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a diagram of culturing conditions used in Example 13.

FIG. 1B is a plot of viable cell density (VCD) vs. culture time of data in Example 13.

FIG. 1C is a plot of percent viability vs. culture time of data in Example 13.

FIG. 1D is a plot of glucose concentration (g/L) vs. culture time of data in Example 13.

FIG. 1E is a plot of lactate concentration (g/L) vs. culture time of data in Example 13.

FIG. 1F is a plot of osmolality (mOsm) vs. culture time of data in Example 13.

FIG. 2A is a diagram of culturing conditions used in Example 16.

FIG. 2B is a plot of viable cell density (VCD) vs. culture time of data in Example 16.

FIG. 2C is a plot of glucose concentration (g/L) vs. culture time of data in Example 16.

FIG. 2D is a plot of ammonium concentration (mmol/L) vs. culture time of data in Example 16.

FIG. 2E is a bar plot of product titer produced in a 0.8× Feed 4 bioreactor, a 1.0× Feed 4 bioreactor, and a control Feed 1 bioreactor over time.

FIG. 3A is a diagram of culturing conditions used in Example 17.

FIG. 3B is a plot of viable cell density (VCD) vs. culture time for all six conditions in Example 17.

FIG. 3C is a plot of cell viability vs. culture time for all six conditions in Example 17.

FIG. 3D is VCD vs. culture time for the bioreactor conditions in Example 17.

FIG. 3E is a plot of VCD vs. culture time for the shake flask conditions in Example 17.

FIG. 3F is a plot of lactate concentration (g/L) vs. culture time for the shake flask conditions in Example 17.

FIG. 3G is a bar plot of product titer produced in a HyClone™ Switch bioreactor, a HyClone™ Adapted bioreactor, and a control Feed 1 bioreactor over time.

FIG. 4A is a diagram of culturing conditions used in Example 18.

FIG. 4B is a plot of viable cell density (VCD) vs. culture time for all six conditions in Example 18.

FIG. 4C is a plot of percent viability vs. culture time for all six conditions in Example 18.

FIG. 4D is a plot of glucose concentration (g/L) vs. culture time for the bioreactor conditions in Example 18.

FIG. 4E is a plot of VCD vs. culture time for the bioreactor conditions in Example 18.

FIG. 4F is a plot of lactate concentration (g/L) vs. culture time for the bioreactor conditions in Example 18.

FIG. 4G is a plot of ammonium concentration (mmol/L) vs. culture time for the bioreactor conditions in Example 18.

FIG. 4H is a bar plot of product titer produced in an Eff B bioreactor, an Eff B 2 bioreactor, and a control Feed 1 bioreactor over time.

FIG. 5A is a plot of viable cell density (VCD) vs. culture time in Example 23.

FIG. 5B is a plot of percent viability vs. culture time in Example 23.

FIG. 5C is a plot of ammonium concentration (mmol/L) vs. culture time in Example 23.

FIG. 5D is a bar plot of product titer for 0.8× F4 (white), 1× F4 (black), and control CDC4 F1 (striped) cultures over time.

FIG. 6A is a plot of viable cell density (VCD) vs. culture time for all conditions in Example 24.

FIG. 6B is a plot of percent viability vs. culture time for all conditions in Example 24.

FIG. 6C is a plot of viable cell density (VCD) vs. culture time for conditions with Hyclone ActiPro or CD-C4 control media in Example 24.

FIG. 6D is a plot of percent viability vs. culture time for conditions with Hyclone ActiPro or CD-C4 control media in Example 24.

FIG. 6E is a plot of osmolality (mOsm) vs. culture time for conditions with Hyclone ActiPro or CD-C4 control media in Example 24.

FIG. 6F is a plot of ammonium concentration (mmol/L) vs. culture time for conditions with Hyclone ActiPro or CD-C4 control media in Example 24.

FIG. 6G is a plot of lactate concentration (g/L) vs. culture time for conditions with Hyclone ActiPro or CD-C4 control media in Example 24.

FIG. 6H is a bar plot of product titers over time in for conditions with Hyclone ActiPro or CD-C4 control media.

### DETAILED DESCRIPTION

Provided herein are methods of producing a recombinant protein that include: (a) providing a cell culture comprising a mammalian cell (e.g., a CHO cell) in a liquid culture medium (e.g., a chemically-defined, animal component-free liquid culture medium, e.g., HyClone™ ActiPro™ or CD-C4), where the CHO cell includes a nucleic acid encoding a recombinant protein; (b) fed-batch culturing the cell culture of step (a) under conditions sufficient for the CHO cell to produce the recombinant protein, where: the fed-batch culturing includes adding a volume of a first feed culture medium (e.g., a chemically-defined, animal component-free liquid culture medium, e.g., about 0.8× to about 1.0× BalanCD′ CHO Feed 4) on about day 2 to about day 5 of the culture and adding a volume of a second feed culture medium (e.g., a chemically-defined, animal component-free liquid culture medium, e.g., about 0.9× to about 1.1× BalanCD™ CHO Feed 2) on about day 6 to about day 13; and (c) recovering the recombinant protein from the mammalian cell or the liquid culture medium. In some embodiments of these methods, the liquid culture medium, the first feed culture medium, and the second feed culture medium are each different.

In some embodiments of these methods, the volume of the first feed culture medium added on about day 2 to about day 5 (e.g., about day 2 to about day 4, about day 2 to about day 3, about day 3 to about day 5, about day 3 to about day 4, or about day 4 to about day 5) is about 4.0% to about 10% (e.g., about 4.0% to about 9.5%, about 4.0% to about 9.0%, about 4.0% to about 8.5%, about 4.0% to about 8.0%, about 4.0% to about 7.5%, about 4.0% to about 7.5%, about 4.0% to about 7.0%, about 4.0% to about 6.5%, about 4.0% to about 6.0%, about 4.0% to about 5.5%, about 4.0% to about 5.0%, about 4.0% to about 4.5%, about 4.5% to about 10%, about 4.5% to about 9.5%, about 4.5% to about 9.0%, about 4.5% to about 8.5%, about 4.5% to about 8.0%, about 4.5% to about 7.5%, about 4.5% to about 7.0%, about 4.5% to about 6.5%, about 4.5% to about 6.0%, about 4.5% to about 5.5%, about 4.5% to about 5.0%, about 5.0% to about 10%, about 5.0% to about 9.5%, about 5.0% to about 9.0%, about 5.0% to about 8.5%, about 5.0% to about 8.0%, about 5.0% to about 7.5%, about 5.0% to about 7.0%, about 5.0% to about 6.5%, about 5.0% to about 6.0%, about 5.0% to about 5.5%, about 5.5% to about 10.0%, about 5.5% to about 9.5%, about 5.5% to about 9.0%, about 5.5% to about 8.5%, about 5.5% to about 8.0%, about 5.5% to about 7.5%, about 5.5% to about 7.0%, about 5.5% to about 6.5%, about 5.5% to about 6.0%, about 6.0% to about 10.0%, about 6.0% to about 9.5%, about 6.0% to about 9.0%, about 6.0% to about 8.5%, about 6.0% to about 8.0%, about 6.0% to about 7.5%, about 6.0% to about 7.0%, about 6.0% to about 6.5%, about 6.5% to about 10.0%, about 6.5% to about 9.5%, about 6.5% to about 9.0%, about 6.5% to about 8.5%, about 6.5% to about 8.0%, about 6.5% to about 7.5%, about 6.5% to about 7.0%, about 7.0% to about 10.0%, about 7.0% to about 9.5%, about 7.0% to about 9.0%, about 7.0% to about 8.5%, about 7.0% to about 8.0%, about 7.0% to about 7.5%, about 7.5% to about 10.0%, about 7.5% to about 9.5%, about 7.5% to about 9.0%, about 7.5% to about 8.5%, about 7.5% to about 8.0%, about 8.0% to about 10.0%, about 8.0% to about 9.5%, about 8.0% to about 9.0%, about 8.0% to about 8.5%, about 8.5% to about 10.0%, about 8.5% to about 9.5%, about 8.5% to about 9.0%, about 9.0% to about 10.0%, about 9.0% to about 9.5%, or about 9.5% to about 10.0%) of the volume of the cell culture in step (a) per day.

In some embodiments, the volume of the second feed culture medium added on about day 6 to about day 13 (e.g., about day 6 to about day 12, about day 6 to about day 11, about day 6 to about day 10, about day 6 to about day 9, about day 6 to about day 8, about day 6 to about day 7, about day 7 to about day 13, about day 7 to about day 12, about day 7 to about day 11, about day 7 to about day 10, about day 7 to about day 9, about day 7 to about day 8, about day 8 to about day 13, about day 8 to about day 12, about day 8 to about day 11, about day 8 to about day 10, about day 8 to about day 9, about day 9 to about day 13, about day 9 to about day 12, about day 9 to about day 11, about day 9 to about day 10, about day 10 to about day 13, about day 10 to about day 12, about day 10 to about day 11, about day 11 to about day 13, about day 11 to about day 12, or about day 12 to about day 13) is about 4.0% to about 10% (e.g., e.g., about 4.0% to about 9.5%, about 4.0% to about 9.0%, about 4.0% to about 8.5%, about 4.0% to about 8.0%, about 4.0% to about 7.5%, about 4.0% to about 7.5%, about 4.0% to about 7.0%, about 4.0% to about 6.5%, about 4.0% to about 6.0%, about 4.0% to about 5.5%, about 4.0% to about 5.0%, about 4.0% to about 4.5%, about 4.5% to about 10%, about 4.5% to about 9.5%, about 4.5% to about 9.0%, about 4.5% to about 8.5%, about 4.5% to about 8.0%, about 4.5% to about 7.5%, about 4.5% to about 7.0%, about 4.5% to about 6.5%, about 4.5% to about 6.0%, about 4.5% to about 5.5%, about 4.5% to about 5.0%, about 5.0% to about 10%, about 5.0% to about 9.5%, about 5.0% to about 9.0%, about 5.0% to about 8.5%, about 5.0% to about 8.0%, about 5.0% to about 7.5%, about 5.0% to about 7.0%, about 5.0% to about 6.5%, about 5.0% to about 6.0%, about 5.0% to about 5.5%, about 5.5% to about 10.0%, about 5.5% to about 9.5%, about 5.5% to about 9.0%, about 5.5% to about 8.5%, about 5.5% to about 8.0%, about 5.5% to about 7.5%, about 5.5% to about 7.0%, about 5.5% to about 6.5%, about 5.5% to about 6.0%, about 6.0% to about 10.0%, about 6.0% to about 9.5%, about 6.0% to about 9.0%, about 6.0% to about 8.5%, about 6.0% to about 8.0%, about 6.0% to about 7.5%, about 6.0% to about 7.0%, about 6.0% to about 6.5%, about 6.5% to about 10.0%, about 6.5% to about 9.5%, about 6.5% to about 9.0%, about 6.5% to about 8.5%, about 6.5% to about 8.0%, about 6.5% to about 7.5%, about 6.5% to about 7.0%, about 7.0% to about 10.0%, about 7.0% to about 9.5%, about 7.0% to about 9.0%, about 7.0% to about 8.5%, about 7.0% to about 8.0%, about 7.0% to about 7.5%, about 7.5% to about 10.0%, about 7.5% to about 9.5%, about 7.5% to about 9.0%, about 7.5% to about 8.5%, about 7.5% to about 8.0%, about 8.0% to about 10.0%, about 8.0% to about 9.5%, about 8.0% to about 9.0%, about 8.0% to about 8.5%, about 8.5% to about 10.0%, about 8.5% to about 9.5%, about 8.5% to about 9.0%, about 9.0% to about 10.0%, about 9.0% to about 9.5%, or about 9.5% to about 10.0%) of the volume of the cell culture of step (a) per day.

### Mammalian Cells

The methods provided herein can be used to culture a variety of different mammalian cells. Non-limiting examples of mammalian cells that can be cultured using any of the methods described herein include: Chinese hamster ovary (CHO) cells (e.g., CHO DG44 cells, CHO-K1s cells, C02.31 clonal cells, A14.13 clonal cells, C02.57 clonal cells, and F05.43 clonal cells), Sp2.0, myeloma cells (e.g., NS/0), B-cells, hybridoma cells, T-cells, human embryonic kidney (HEK) cells (e.g., HEK 293E and HEK 293F), African green monkey kidney epithelial cells (Vero) cells, and Madin-Darby Canine (Cocker Spaniel) kidney epithelial cells (MDCK) cells. Additional mammalian cells that can be cultured using the methods described herein are known in the art.

The mammalian cell can contain a recombinant nucleic acid (e.g., a nucleic acid stably integrated in the mammalian cell's genome) that encodes a recombinant protein (e.g., a fusion protein, an antibody or antibody fragment).

### Recombinant Protein

Non-limiting examples of recombinant proteins produced by the methods provided herein include immunoglobulins (including light and heavy chain immunoglobulins), antibodies, or antibody fragments (e.g., any of the antibody fragment described herein). Non-limiting examples of recombinant proteins that can be produced by the methods described herein include ranibizumab and bevacizumab.

In some embodiments, the recombinant protein in non-glycosylated. In some embodiments, the recombinant protein is an antibody or an antigen-binding antibody fragment. In some embodiments, the recombinant protein can be CroFab®, DigiFab®, Digibind®, ReoPro®, and Cimzia®. In some embodiments, the recombinant protein can be, e.g., TOB5-D4, LA13-IIE3, anti-MUC1, SH363-A9, SH365-C9, filgrastim (Neupogen), pegfilgrastim (Neulasta), insulin ((e.g. insulin glargine (Lantus), insulin aspart, insulin glulisine, insulin lispro (fast-acting insulin analog), insulin detemir (long-acting insulin), isophane insulin (intermediate-acting insulin)), insulin-like growth factor 1 (Mecasermin), insulin-like growth factor I and its binding protein IGFBP-3 (Mecasermin rinfabate), denileukin diftitox, endostatin, interleukin-2 (Aldesleukin), interleukin-1 (IL1) receptor antagonist, interleukin-11, interferon alpha-2a, interferon alpha-2b, interferon alpha-1b, interferon beta-1b, interferon gamma-1a, interferon gamma-1b, tasonermin, molgramostim, nartograstim, palifermin, sargramostim, salmon calcitonin, glucagon, glucagon like peptide 1 (Liraglutide), bacterial carboxypeptidase G2 (Glucarpidase), B-type natriuretic peptide, OspA (Outer surface protein A fragment from Borrelia burgdorferi), palifermin (truncade keratinocyte growth factor), parathyroid hormone, growth hormone, pegvisomant (modified GH; Somavert), reteplase (plasminogen activator; Rapilysi), somatropin (tasonermin; Humatrope), tasonermin (cytokine), urate oxidase, teriparatide (parathyroid hormone), albumin, Hepatitis B surface antigen, Hepatitis B surface antigen and hepatitis A virus inactivated, hirudine, HPV vaccine, HPV surface antigens, platelet derived growth factor-BB, rasburicase, sargramostim, cytochromes (e.g. P450 enzymes), interferon, leptin, and brolucizumab. In some embodiments, the recombinant protein can be an antibody or an antigen-binding antibody fragment selected from the group of: abciximab, abituzumab, abrezekimab, abrilumab, actoxumab, adalimumab, adecatumumab, atidortoxumab, aducanumab, afasevikumab, alacizumab pegol, alemtuzumab, alirocumab, amatuximab, andecaliximab, anetumab ravtansine, anifrolumab, anrukinzumab, apolizumab, aprutumab ixadotin, ascrinvacumab, aselizumab, atezolizumab, atinumab, atorolimumab, avelumab, azintuxizumab vedotin, bapineuzumab, basiliximab, bavituximab, BCD-100, belantamab mafodotin, belimumab, bemarituzumab, benralizumab, berlimatoxumab, bermekimab, bersanlimab, bertilimumab, bevacizumab, bezlotoxumab, bimagrumab, bimekizumab, birtamimab, bivatuzumab mertansine, bleselumab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, carlumab, carotuximab, cBR96, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, crenezumab, crizanlizumab, crotedumab, CR6261, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, dezamizumab, dinutuximab, diridavumab, domagrozumab, dostarlimab, drozitumab, DS-8201, duligotuzumab, dupilumab, durvalumab, dusigitumab, duvortuxizumab, ecromeximab, eculizumab, efalizumab, efungumab, eldelumab, elezanumab, elgemtumab, elotuzumab, emactuzumab, emapalumab, emibetuzumab, emicizumab, enapotamab vedotin, enavatuzumab, enfortumab vedotin, enoblituzumab, enokizumab, enoticumab, ensituximab, epratuzumab, eptinezumab, erenumab, erlizumab, etaracizumab, etigilimab, etrolizumab, evinacumab, evolocumab, exbivirumab, faricimab, farletuzumab, fasinumamb, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, fulranumab, futuximab, galcanezumab, galiximab, gancotamab, ganitumab, gantenerumab, gatipotuzumab, gedivumab, gemtuzumab ozogamicin, gevokizumab, gimsilumab, girentuximab, glembatumumab vedotin, golimumab, gomiliximab, gosuranemab, guselkumab, ianalumab, ibalizumab, IBI308, icrucumab, idarucizumab, ifabotuzumab, iladatuzumab vedotin, IMAB362, imalumab, imaprelimab, imgatuzumab, inclacumab, indatuximab ravtansine, indusatumab vedotin, inebilizumab, infliximab, intelumumab, inotuzumab ozogamicin, ipilimumab, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lendalizumab, lenvervimab, lenzilumab, lerdelimumab, leronlimab, lesolimab, letolizumab, lexatumumab, libivirumab, lifastuzumab vedotin, ligelizumab, loncastuximab tesirine, losatuxizumab vedotin, lintuzumab, lirilumab, lodelcizumab, lorvotuzumab mertansine, lucatumumab, lulizumab, lumiliximab, lumretuzumab, lupartumab amadotin, lutikizumab, mapatumumab, margetuximab, marstacimab, maslimomab, mavrilimumab, matuzumab, mepolizumab, metelimumab, milatuzumab, mirikizumab, mirvetuximab soravtansine, modotuximab, mogamulizumab, monalizumab, morolimumab, mosunetuzumab, motavizumab, namilumab, naratuximab emtansine, narnatumab, natalizumab, navicixizumab, navivumab, naxitamab, nebacumab, necitumumab, nemolizumab, NEOD001, nesvacumab, netakimab, nimotuzumab, nirsevimab, nivolumab, obiltoxaximab, obinutuzumab, ocaratuzumab, ocrelizumab, ofatumumab, olaratumab, oleclumab, olendalizumab, olokizumab, omalizumab, OMS721, onartuzumab, onartuzumab, ontuxizumab, onvatilimab, opicinumab, oportuzumab monatox, orticumab, otelixizumab, otilimab, otlertuzumab, oxelumab, ozanezumab, ozoralizumab, pagibaximab, palivizumab, pmrevlumab, panitumumab, pankomab, panobacumab, parsatuzumab, pascolizumab, pasotuxizumab, pateclizumab, patritumab, PDR001, pembrolizumab, perakizumab, pertuzumab, pexelizumab, pidilizumab, pinatuzumab vedotin, placulumab, plozalizumab, pogalizumab, polatuzumab vedotin, ponezumab, porgaviximab, prasinezumab, prezalizumab, priliximab, pritoxaximab, pritumumab, PRO 140, quilizumab, radretumab, rafivirumab, ralpancizumab, ramucirumab, ranibizumab, raxibacumab, ravagalimab, ravulizumab, refanezumab, regavirumab, relatlimab, remtolumab, reslizumab, rilotumumab, rinucumab, risankizumab, rituximab, rivabazumab pegol, robatumumab, Rmab, roledumab, romikimab, romosozumab, rontalizumab, rosmantuzumab, rovalptuzumab tesirine, rovelizumab, rozanolixizumab, ruplizumab, SA237, sacituzumab govitecan, samalizumab, samrotamab vedotin, sarilumab, satralizumab, secukinumab, selicrelumab, seribantumab, setoxaximab, setrusumab, sevirumab, sibrotuzumab, SGN-CD19A, SHP647, sifalimumab, siltuximab, simtuzumab, siplizumab, sirtratumab vedotin, sirukumab, sofituzumab vedotin, solanezumab, sonepcizumab, sontuzumab, spartalizumab, stamulumab, suptavumab, sutimlimab, suvizumab, suvratoxumab, tabalumab, tacatuzumab tetraxetan, tadocizumab, talizumab, tanezumab, tarextumab, tavolimab, tefibazumab, telisotuzumab vedotin, teneliximab, teplizumab, tepoditamab, teprotumumab, tesidolumab, tetulomab, tezepelumab, TGN1412, tibulizumab, tildrakizumab, tigatuzumab, timigutuzumab, timolumab, tiragotumab, tiselizumab, tisotumab vedotin, TNX-650, tocilizumab, tomuzotuximab, toralizumab, tosatoxumab, tositumomab, tovetumab, tralokinumab, trastuzumab, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandotuzumab vedotin, vantictumab, vanucizumab, vapaliximab, varisacumab, varlilumab, vatelizumab, vedolizumab, veltuzumab, vesencumab, visilizumab, vobarilizumab, volociximab, vonterolizumab, vopratelimab, vorsetuzumab, votumumab, vunakizumab, xentuzumab, XMAB-5574, zalutumumab, zanolimumab, zatuximab, zenocutuzumab, ziralimumab, and zolbetuximab, and an antigen-binding antibody fragment of any of these antibodies. In some embodiments, the recombinant protein is an antibody or antigen-binding antibody fragment that binds to vascular endothelial growth factor (VEGF). In some embodiments, the antigen-binding antibody fragment is ranibizumab.

In some embodiments, the recombinant protein has an amino acid sequence that differs from a reference protein. For example, the reference protein is ranibizumab and the recombinant protein has a conservative amino acid substitution for one or more of the amino acids of ranibizumab.

In some embodiments, the recombinant protein includes an amino acid sequence that is at least 80% (e.g., at least 81%, at least 82%, at least 83%, at least 84%, at least 85%, at least 86%, at least 88%, at least 90%, at least 92%, at least 94%, at least 95%, at least 96%, at least 97%, at least 98%, at least 99%, at least 99.5%, or 100%) identical to SEQ ID NO: 1. In some embodiments, the recombinant protein includes an amino acid sequence that is at least 80% (e.g., at least 81%, at least 82%, at least 83%, at least 84%, at least 85%, at least 86%, at least 88%, at least 90%, at least 92%, at least 94%, at least 95%, at least 96%, at least 97%, at least 98%, at least 99%, at least 99.5%, or 100%) identical to SEQ ID NO: 2.

SEQ ID NO: 1
DIQLTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTV
SEQ ID NO: 2
SGGGSGSGDFDYEKMANANKGAMTENADENALQSDAKGKLDSVATDYGA
AIDGFIGDVSGLANGNGATGDFAGSNSQMAQVGDGDNSPLMNNFRQYLP
SLPQSVECRPFVFSAGKPYEFSIDCDKINLFRGVFAFLLYVATFMYVFS
TFANILRNKES

In some embodiments, the recombinant protein includes a sequence that differs from the amino sequence of SEQ ID NO: 1 by one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30) amino acids. In some embodiments, the recombinant protein includes a sequence that differs from the amino sequence of SEQ ID NO: 2 by one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30) amino acids.

For example, where a recombinant protein includes a sequence that differs from the amino acid sequence of SEQ ID NO: 1 and/or SEQ ID NO: 2 by one or more amino acids, the amino acid present in SEQ ID NO: 1 and/or SEQ ID NO: 2 can be replaced by a similar amino acid. For example, a serine can be replaced by any of glycine, alanine, serine, threonine, or proline; arginine can be replaced by asparagine, lysine, glutamine, or histidine; leucine can be replaced by phenylalanine, isoleucine, valine, or methionine; proline can be replaced with glycine, alanine, serine, or threonine; alanine can be replaced with glycine, threonine, proline, or serine; valine can be replaced with methionine, phenylalanine, isoleucine, or leucine; glycine can be replaced with alanine, threonine, proline, or serine; isoleucine can be replaced with phenylalanine, valine, leucine, or methionine; phenylalanine can be replaced with tryptophan or tyrosine; tyrosine can be replaced with tryptophan or phenylalanine; cysteine can be replaced with serine or threonine; histidine can be replaced with asparagine, lysine, glutamine, or arginine; glutamine can be replaced with glutamic acid, asparagine, or aspartic acid; asparagine can be replaced with glutamic acid, aspartic acid, or glutamine; lysine can be replaced with asparagine, glutamine, arginine, or histidine; asparatic acid can be replaced with glutamic acid, asparagine, or glutamine; glutamic acid can be replaced by asparagine, aspartic acid, or glutamine; methionine can be replaced with phenylalanine, isoleucine, valine, or leucine; and tryptophan can be replaced with phenylalanine or tyrosine.

In some examples, a precursor form of the recombinant protein can include a signal sequence.

In some embodiments, a secreted recombinant protein is recovered and optionally purified from the recombinant protein production medium (e.g., using any of the exemplary methods known in the art)

In some embodiments, at least about 30% (e.g., at least about 40%, at least about 50%, at least about 55%, at least about 60%, at least about 65%, at least about 70%, at least about 75%, at least about 80%, at least about 85%, at least about 90%, at least about 95%, or at least about 99%) of the secreted recombinant protein is properly folded or unfolded in the recombinant protein production medium.

In some embodiments, less than about 30% (e.g., less than about 25%, less than about 20%, less than about 15%, less than about 10%, less than about 8%, less than about 6%, less than about 4%, less than about 2%, or less than about 1%) of the secreted recombinant protein is not properly folded or unfolded in the recombinant protein production medium. The term “immunoglobulin,” as used herein comprises a polypeptide containing an amino acid sequence of at least 15 amino acids (e.g., at least 20, 30, 40, 50, 60, 70, 80, 90, or 100 amino acids) of an immunoglobulin protein (e.g., a variable domain sequence, a framework sequence, or a constant domain sequence). In some embodiments, the immunoglobulin comprises at least 15 amino acids of a light chain immunoglobulin and at least 15 amino acids of a heavy chain immunoglobulin. In some embodiments, the immunoglobulin is an isolated antibody (e.g., an IgG, IgE, IgD, IgA, or IgM). In some embodiments, the immunoglobulin is a subclass of IgG (e.g., IgG1, IgG2, IgG3, or IgG4). In some embodiments, the immunoglobulin is a mouse, chimeric, humanized, or human antibody. In some embodiments, the immunoglobulin is an antibody fragment, e.g., a Fab fragment, a F(ab′)₂ fragment, or a scFv fragment. In some embodiments, the immunoglobulin is a bi-specific antibody or a tri-specific antibody, or a dimer, trimer, or multimer antibody, or a diabody, an Affibody®, or a Nanobody®. In some embodiments, the immunoglobulin is an engineered protein containing at least one immunoglobulin domain (e.g., a fusion protein). Non-limiting examples of immunoglobulins are described herein and additional examples of immunoglobulins are described in the art.

### Culture Media

Liquid culture media are known in the art. In some embodiments, the liquid culture medium, the first feed medium, and/or the second feed medium can be a chemically-defined liquid culture medium, an animal-derived component free liquid culture medium, a serum-free liquid culture medium, or a serum-containing liquid culture medium. In some examples, one or more (e.g., one, two, or three) of the liquid culture medium, the first feed medium, and the second feed medium are a chemically-defined, animal component-free liquid culture medium. In some examples, each of the liquid culture medium, the first feed medium, and the second feed medium are different chemically-defined animal component-free liquid culture medium. Non-limiting examples of chemically-defined liquid culture media, animal-derived component free liquid culture media, serum-free liquid culture media, and serum-containing liquid culture media are commercially available.

A liquid culture medium typically contains an energy source (e.g., a carbohydrate, such as glucose), essential amino acids (e.g., the basic set of twenty amino acids plus cysteine), vitamins and/or other organic compounds required at low concentrations, free fatty acids, and/or trace elements. The first and/or second liquid culture medium can, if desired, be supplemented with, e.g., a mammalian hormone or growth factor (e.g., insulin, transferrin, or epidermal growth factor), salts and buffers (e.g., calcium, magnesium, and phosphate salts), nucleosides and bases (e.g., adenosine, thymidine, and hypoxanthine), protein and tissue hydrolysates, and/or any combination of these additives.

Non-limiting examples of liquid culture media that are particularly useful in the presently described methods include, e.g., CD-C4, BalanCD™ CHO Feed 2, BalanCD™ CHO Feed 4, and HyClone™ ActiPro™.

CD-C4 (available for purchase from suppliers such as ThermoFisher Scientific) is a a chemically-defined, animal component-free liquid culture medium that is also serum-free, protein-free, lacking thymidine, hypoxanthine, glutamine, and phenol red. It has a low endotoxin level, and does not include any antibiotics. It includes, among other components, sodium bicarbonate and sodium pyruvate

BalanCD™ CHO Feed 4, (available for purchase from suppliers such as Fujifilm Irvine Scientific) is a a chemically-defined, animal component-free liquid culture medium described in, e.g., United States Food and Drug Administration (FDA) Drug Master File (DMF) DMF31306 (powder).

BalanCD™ CHO Feed 2, (available for purchase from suppliers such as Fujifilm Irvine Scientific) is a chemically-defined, animal component-free liquid culture medium described in FDA DMF26574 (liquid) or DMF26522 (powder)).

HyClone™ ActiPro™, (available for purchase from suppliers such as VWR) is a a chemically-defined, animal component-free liquid culture medium that does not contain growth factors such as insulin, peptides, hydrolysates, phenol red, or 2-mercaptoethanol).

In some embodiments, the liquid culture medium includes HyClone™ ActiPro™ or CD-C4.

In some embodiments, the first feed medium comprises about 0.7× to about 1.2× (e.g., about 0.7× to about 1.1×, about 0.7× to about 1.0×, about 0.7× to about 0.9×, about 0.7× to about 0.8×, about 0.8× to about 1.2×, about 0.8× to about 1.1×, about 0.8× to about 1.0×, about 0.8× to about 0.9×, about 0.9× to about 1.2×, about 0.9× to about 1.1×, about 0.9× to about 1.0×, about 1.0× to about 1.2×, about 1.0× to about 1.1×, or about 1.1× to about 1.2×) BalanCD™ CHO Feed 4. In some embodiments, the first feed culture medium comprises about 0.8× BalanCD™ CHO Feed 4.

In some embodiments, the second feed medium comprises about 0.7× to about 1.2× (e.g., about 0.7× to about 1.1×, about 0.7× to about 1.0×, about 0.7× to about 0.9×, about 0.7× to about 0.8×, about 0.8× to about 1.2×, about 0.8× to about 1.1×, about 0.8× to about 1.0×, about 0.8× to about 0.9×, about 0.9× to about 1.2×, about 0.9× to about 1.1×, about 0.9× to about 1.0×, about 1.0× to about 1.2×, about 1.0× to about 1.1×, or about 1.1× to about 1.2×) BalanCD™ CHO Feed 2. In some embodiments, the second feed culture medium comprises about 1.0× BalanCD™ CHO Feed 2.

In some embodiments, one or more (e.g., one, two, or three) of the first liquid culture medium, the first feed culture medium, and the second feed culture medium further include a concentration of N-acetylglucosamine sufficient to maintain a concentration of N-acetylglucosamine in the cell culture of about 2 mM to about 8 mM (e.g., about 2.0 mM to about 7.5 mM, about 2.0 mM to about 7.0 mM, about 2.0 mM to about 6.5 mM, about 2.0 mM to about 6.0 mM, about 2.0 mM to about 5.5 mM, about 2.0 mM to about 5.0 mM, about 2.0 mM to about 4.5 mM, about 2.0 mM to about 4.0 mM, about 2.0 mM to about 3.5 mM, about 2.0 mM to about 3.0 mM, about 2.0 mM to about 2.5 mM, about 2.5 mM to about 8.0 mM, about 2.5 mM to about 7.5 mM, about 2.5 mM to about 7.0 mM, about 2.5 mM to about 6.5 mM, about 2.5 mM to about 6.0 mM, about 2.5 mM to about 5.5 mM, about 2.5 mM to about 5.0 mM, about 2.5 mM to about 4.5 mM, about 2.5 mM to about 4.0 mM, about 2.5 mM to about 3.5 mM, about 2.5 mM to about 3.0 mM, about 3.0 mM to about 8.0 mM, about 3.0 mM to about 7.5 mM, about 3.0 mM to about 7.0 mM, about 3.0 mM to about 6.5 mM, about 3.0 mM to about 6.0 mM, about 3.0 mM to about 5.5 mM, about 3.0 mM to about 5.0 mM, about 3.0 mM to about 4.5 mM, about 3.0 mM to about 4.0 mM, about 3.0 mM to about 3.5 mM, about 3.5 mM to about 8.0 mM, about 3.5 mM to about 8.0 mM, about 3.5 mM to about 7.5 mM, about 3.5 mM to about 7.0 mM, about 3.5 mM to about 6.5 mM, about 3.5 mM to about 6.0 mM, about 3.5 mM to about 5.5 mM, about 3.5 mM to about 5.0 mM, about 3.5 mM to about 4.5 mM, about 3.5 mM to about 4.0 mM, about 4.0 mM to about 8.0 mM, about 4.0 mM to about 7.5 mM, about 4.0 mM to about 7.0 mM, about 4.0 to about 6.5 mM, about 4.0 mM to about 6.0 mM, about 4.0 mM to about 5.5 mM, about 4.0 mM to about 5.0 mM, about 4.0 mM to about 4.5 mM, about 4.5 mM to about 8.0 mM about 4.5 mM to about 7.5 mM, about 4.5 mM to about 7.0 mM, about 4.5 mM to about 6.5 mM, about 4.5 mM to about 6.0 mM, about 4.5 mM to about 5.5 mM, about 4.5 mM to about 5.0 mM, about 5.0 mM to about 8.0 mM, about 5.0 mM to about 7.5 mM, about 5.0 mM to about 7.0 mM, about 5.0 mM to about 6.5 mM, about 5.0 mM to about 6.0 mM, about 5.0 mM to about 5.5 mM, about 5.5 mM to about 8.0 mM, about 5.5 mM to about 7.5 mM, about 5.5 mM to about 7.0 mM, about 5.5 mM to about 6.5 mM, about 5.5 mM to about 6.0 mM, about 6.0 mM to about 8.0 mM, about 6.0 mM to about 7.5 mM, about 6.0 mM to about 7.0 mM, about 6.0 mM to about 6.5 mM, about 6.5 mM to about 8.0 mM, about 6.5 mM to about 7.5 mM, about 6.5 mM to about 7.0 mM, about 7.0 mM to about 8.0 mM, about 7.0 mM to about 7.5 mM, or about 7.5 mM to about 8.0 mM).

In some embodiments, the first feed culture medium and/or the second feed culture medium further include about 4 g/L to about 6 g/L (e.g., about 4.0 g/L to about 5.8 g/L, about 4.0 g/L to about 5.6 g/L, about 4.0 g/L to about 5.4 g/L, about 4.0 g/L to about 5.2 g/L, about 4.0 g/L to about 5.0 g/L, about 4.0 g/L to about 4.8 g/L, about 4.0 g/L to about 4.6 g/L, about 4.0 g/L to about 4.4 g/L, about 4.0 g/L to about 4.2 g/L, about 4.2 g/L to about 6.0 g/L, about 4.2 g/L to about 5.8 g/L, about 4.2 g/L to about 5.6 g/L, about 4.2 g/L to about 5.4 g/L, about 4.2 g/L to about 5.2 g/L, about 4.2 g/L to about 5.0 g/L, about 4.2 g/L to about 4.8 g/L, about 4.2 g/L to about 4.6 g/L, about 4.2 g/L to about 4.4 g/L, about 4.4 g/L to about 6.0 g/L, about 4.4 g/L to about 5.8 g/L, about 4.4 g/L to about 5.6 g/L, about 4.4 g/L to about 5.4 g/L, about 4.4 g/L to about 5.2 g/L, about 4.4 g/L to about 5.0 g/L, about 4.4 to about 4.8 g/L, about 4.4 g/L to about 4.6 g/L, about 4.6 g/L to about 6.0 g/L, about 4.6 g/L to about 5.8 g/L, about 4.6 g/L to about 5.6 g/L, about 4.6 g/L to about 5.4 g/L, about 4.6 g/L to about 5.2 g/L, about 4.6 g/L to about 5.0 g/L, about 4.6 g/L to about 4.8 g/L, about 4.8 g/L to about 6.0 g/L, about 4.8 g/L to about 5.8 g/L, about 4.8 g/L to about 5.6 g/L, about 4.8 g/L to about 5.4 g/L, about 4.8 g/L to about 5.2 g/L, about 4.8 g/L to about 5.0 g/L, about 5.0 g/L to about 6.0 g/L, about 5.0 g/L to about 5.8 g/L, about 5.0 g/L to about 5.6 g/L, about 5.0 g/L to about 5.4 g/L, about 5.0 g/L to about 5.2 g/L, about 5.2 g/L to about 6.0 g/L, about 5.2 g/L to about 5.8 g/L, about 5.2 g/L to about 5.6 g/L, about 5.2 g/L to about 5.4 g/L, about 5.4 g/L to about 6.0 g/L, about 5.4 g/L to about 5.8 g/L, about 5.4 g/L to about 5.6 g/L, about 5.6 g/L to about 6.0 g/L, about 5.6 g/L to about 5.8, or about 5.8 g/L to about 6.0 g/L) of glucose.

Additional examples of liquid tissue culture medium and medium components are known in the art.

Skilled practitioners will appreciate that the first liquid culture medium and the second liquid culture medium described herein can be the same type of media or different media.

Vessel

The cell culture can be disposed or contained in any suitable vessel known in the art. For example, the vessel can be a bioreactor having an internal volume of about 100 mL to about 15,000 L (e.g., about 100 mL to about 12,500 L, about 100 mL to about 10,000 L, about 100 mL to about 8,000 L, about 100 mL to about 6,000 L, about 100 mL to about 5,000 L, about 100 mL to about 4,500 L, about 100 mL to about 4,000 L, about 100 mL to about 3,500 L, about 100 mL to about 3,000 L, about 100 mL to about 2,500 L, about 100 mL to about 2,000 L, about 100 mL to about 1,500 L, about 100 mL to about 1,000 L, about 100 mL to about 500 L, about 100 mL to about 250 L, about 100 mL to about 200 L, about 100 mL to about 150 L, about 100 mL to about 100 L, about 100 mL to about 80 L, about 100 mL to about 60 L, about 100 mL to about 50 L, about 100 mL to about 40 L, about 100 mL to about 30 L, about 100 mL to about 20 L, about 100 mL to about 10 L, about 100 mL to about 5 L, about 100 mL to about 2 L, about 100 mL to about 1 L, about 100 mL to about 750 mL, about 100 mL to about 500 mL, about 100 mL to about 250 mL, about 250 mL to about 15,000 L, about 250 mL to about 12,500 L, about 250 mL to about 10,000 L, about 250 mL to about 8,000 L, about 250 mL to about 6,000 L, about 250 mL to about 5,000 L, about 250 mL to about 4,500 L, about 250 mL to about 4,000 L, about 250 mL to about 3,500 L, about 250 mL to about 3,000 L, about 250 mL to about 2,500 L, about 250 mL to about 2,000 L, about 250 mL to about 1,500 L, about 250 mL to about 1,000 L, about 250 mL to about 500 L, about 250 mL to about 250 L, about 250 mL to about 200 L, about 250 mL to about 150 L, about 250 mL to about 100 L, about 250 mL to about 80 L, about 250 mL to about 60 L, about 250 mL to about 50 L, about 250 mL to about 40 L, about 250 mL to about 30 L, about 250 mL to about 20 L, about 250 mL to about 10 L, about 250 mL to about 5 L, about 250 mL to about 2 L, about 250 mL to about 1 L, about 250 mL to about 750 mL, about 250 mL to about 500 mL, about 500 mL to about 15,000 L, about 500 mL to about 12,500 L, about 500 mL to about 10,000 L, about 500 mL to about 8,000 L, about 500 mL to about 6,000 L, about 500 mL to about 5,000 L, about 500 mL to about 4,500 L, about 500 mL to about 4,000 L, about 500 mL to about 3,500 L, about 500 mL to about 3,000 L, about 500 mL to about 2,500 L, about 500 mL to about 2,000 L, about 500 mL to about 1,500 L, about 500 mL to about 1,000 L, about 500 mL to about 500 L, about 500 mL to about 250 L, about 500 mL to about 200 L, about 500 mL to about 150 L, about 500 mL to about 100 L, about 500 mL to about 80 L, about 500 mL to about 60 L, about 500 mL to about 50 L, about 500 mL to about 40 L, about 500 mL to about 30 L, about 500 mL to about 20 L, about 500 mL to about 10 L, about 500 mL to about 5 L, about 500 mL to about 2 L, about 500 mL to about 1 L, about 500 mL to about 750 mL, about 750 mL to about 15,000 L, about 750 mL to about 12,500 L, about 750 mL to about 10,000 L, about 750 mL to about 8,000 L, about 750 mL to about 6,000 L, about 750 mL to about 5,000 L, about 750 mL to about 4,500 L, about 750 mL to about 4,000 L, about 750 mL to about 3,500 L, about 750 mL to about 3,000 L, about 750 mL to about 2,500 L, about 750 mL to about 2,000 L, about 750 mL to about 1,500 L, about 750 mL to about 1,000 L, about 750 mL to about 500 L, about 750 mL to about 250 L, about 750 mL to about 200 L, about 750 mL to about 150 L, about 750 mL to about 100 L, about 750 mL to about 80 L, about 750 mL to about 60 L, about 750 mL to about 50 L, about 750 mL to about 40 L, about 750 mL to about 30 L, about 750 mL to about 20 L, about 750 mL to about 10 L, about 750 mL to about 5 L, about 750 mL to about 2 L, about 750 mL to about 1 L, about 1 L to about 15,000 L, about 1 L to about 12,500 L, about 1 L to about 10,000 L, about 1 L to about 8,000 L, about 1 L to about 6,000 L, about 1 L to about 5,000 L, about 1 L to about 4,500 L, about 1 L to about 4,000 L, about 1 L to about 3,500 L, about 1 L to about 3,000 L, about 1 L to about 2,500 L, about 1 L to about 2,000 L, about 1 L to about 1,500 L, about 1 L to about 1,000 L, about 1 L to about 500 L, about 1 L to about 250 L, about 1 L to about 200 L, about 1 L to about 150 L, about 1 L to about 100 L, about 1 L to about 80 L, about 1 L to about 60 L, about 1 L to about 50 L, about 1 L to about 40 L, about 1 L to about 30 L, about 1 L to about 20 L, about 1 L to about 10 L, about 1 L to about 5 L, about 1 L to about 2 L, about 2 L to about 15,000 L, about 2 L to about 12,500 L, about 2 L to about 10,000 L, about 2 L to about 8,000 L, about 2 L to about 6,000 L, about 2 L to about 5,000 L, about 2 L to about 4,500 L, about 2 L to about 4,000 L, about 2 L to about 3,500 L, about 2 L to about 3,000 L, about 2 L to about 2,500 L, about 2 L to about 2,000 L, about 2 L to about 1,500 L, about 2 L to about 1,000 L, about 2 L to about 500 L, about 2 L to about 250 L, about 2 L to about 200 L, about 2 L to about 150 L, about 2 L to about 100 L, about 2 L to about 80 L, about 2 L to about 60 L, about 2 L to about 50 L, about 2 L to about 40 L, about 2 L to about 30 L, about 2 L to about 20 L, about 2 L to about 10 L, about 2 L to about 5 L, about 5 L to about 15,000 L, about 5 L to about 12,500 L, about 5 L to about 10,000 L, about 5 L to about 8,000 L, about 5 L to about 6,000 L, about 5 L to about 5,000 L, about 5 L to about 4,500 L, about 5 L to about 4,000 L, about 5 L to about 3,500 L, about 5 L to about 3,000 L, about 5 L to about 2,500 L, about 5 L to about 2,000 L, about 5 L to about 1,500 L, about 5 L to about 1,000 L, about 5 L to about 500 L, about 5 L to about 250 L, about 5 L to about 200 L, about 5 L to about 150 L, about 5 L to about 100 L, about 5 L to about 80 L, about 5 L to about 60 L, about 5 L to about 50 L, about 5 L to about 40 L, about 5 L to about 30 L, about 5 L to about 20 L, about 5 L to about 10 L, about 10 L to about 15,000 L, about 10 L to about 12,500 L, about 10 L to about 10,000 L, about 10 L to about 8,000 L, about 10 L to about 6,000 L, about 10 L to about 5,000 L, about 10 L to about 4,500 L, about 10 L to about 4,000 L, about 10 L to about 3,500 L, about 10 L to about 3,000 L, about 10 L to about 2,500 L, about 10 L to about 2,000 L, about 10 L to about 1,500 L, about 10 L to about 1,000 L, about 10 L to about 500 L, about 10 L to about 250 L, about 10 L to about 200 L, about 10 L to about 150 L, about 10 L to about 100 L, about 10 L to about 80 L, about 10 L to about 60 L, about 10 L to about 50 L, about 10 L to about 40 L, about 10 L to about 30 L, about 10 L to about 20 L, about 20 L to about 15,000 L, about 20 L to about 12,500 L, about 20 L to about 10,000 L, about 20 L to about 8,000 L, about 20 L to about 6,000 L, about 20 L to about 5,000 L, about 20 L to about 4,500 L, about 20 L to about 4,000 L, about 20 L to about 3,500 L, about 20 L to about 3,000 L, about 20 L to about 2,500 L, about 20 L to about 2,000 L, about 20 L to about 1,500 L, about 20 L to about 1,000 L, about 20 L to about 500 L, about 20 L to about 250 L, about 20 L to about 200 L, about 20 L to about 150 L, about 20 L to about 100 L, about 20 L to about 80 L, about 20 L to about 60 L, about 20 L to about 50 L, about 20 L to about 40 L, about 20 L to about 30 L, about 30 L to about 15,000 L, about 30 L to about 12,500 L, about 30 L to about 10,000 L, about 30 L to about 8,000 L, about 30 L to about 6,000 L, about 30 L to about 5,000 L, about 30 L to about 4,500 L, about 30 L to about 4,000 L, about 30 L to about 3,500 L, about 30 L to about 3,000 L, about 30 L to about 2,500 L, about 30 L to about 2,000 L, about 30 L to about 1,500 L, about 30 L to about 1,000 L, about 30 L to about 500 L, about 30 L to about 250 L, about 30 L to about 200 L, about 30 L to about 150 L, about 30 L to about 100 L, about 30 L to about 80 L, about 30 L to about 60 L, about 30 L to about 50 L, about 30 L to about 40 L, about 40 L to about 15,000 L, about 40 L to about 12,500 L, about 40 L to about 10,000 L, about 40 L to about 8,000 L, about 40 L to about 6,000 L, about 40 L to about 5,000 L, about 40 L to about 4,500 L, about 40 L to about 4,000 L, about 40 L to about 3,500 L, about 40 L to about 3,000 L, about 40 L to about 2,500 L, about 40 L to about 2,000 L, about 40 L to about 1,500 L, about 40 L to about 1,000 L, about 40 L to about 500 L, about 40 L to about 250 L, about 40 L to about 200 L, about 40 L to about 150 L, about 40 L to about 100 L, about 40 L to about 80 L, about 40 L to about 60 L, about 40 L to about 50 L, about 50 L to about 15,000 L, about 50 L to about 12,500 L, about 50 L to about 10,000 L, about 50 L to about 8,000 L, about 50 L to about 6,000 L, about 50 L to about 5,000 L, about 50 L to about 4,500 L, about 50 L to about 4,000 L, about 50 L to about 3,500 L, about 50 L to about 3,000 L, about 50 L to about 2,500 L, about 50 L to about 2,000 L, about 50 L to about 1,500 L, about 50 L to about 1,000 L, about 50 L to about 500 L, about 50 L to about 250 L, about 50 L to about 200 L, about 50 L to about 150 L, about 50 L to about 100 L, about 50 L to about 80 L, about 50 L to about 60 L, about 60 L to about 15,000 L, about 60 L to about 12,500 L, about 60 L to about 10,000 L, about 60 L to about 8,000 L, about 60 L to about 6,000 L, about 60 L to about 5,000 L, about 60 L to about 4,500 L, about 60 L to about 4,000 L, about 60 L to about 3,500 L, about 60 L to about 3,000 L, about 60 L to about 2,500 L, about 60 L to about 2,000 L, about 60 L to about 1,500 L, about 60 L to about 1,000 L, about 60 L to about 500 L, about 60 L to about 250 L, about 60 L to about 200 L, about 60 L to about 150 L, about 60 L to about 100 L, about 60 L to about 80 L, about 80 L to about 15,000 L, about 80 L to about 12,500 L, about 80 L to about 10,000 L, about 80 L to about 8,000 L, about 80 L to about 6,000 L, about 80 L to about 5,000 L, about 80 L to about 4,500 L, about 80 L to about 4,000 L, about 80 L to about 3,500 L, about 80 L to about 3,000 L, about 80 L to about 2,500 L, about 80 L to about 2,000 L, about 80 L to about 1,500 L, about 80 L to about 1,000 L, about 80 L to about 500 L, about 80 L to about 250 L, about 80 L to about 200 L, about 80 L to about 150 L, about 80 L to about 100 L, about 100 L to about 15,000 L, about 100 L to about 12,500 L, about 100 L to about 10,000 L, about 100 L to about 8,000 L, about 100 L to about 6,000 L, about 100 L to about 5,000 L, about 100 L to about 4,500 L, about 100 L to about 4,000 L, about 100 L to about 3,500 L, about 100 L to about 3,000 L, about 100 L to about 2,500 L, about 100 L to about 2,000 L, about 100 L to about 1,500 L, about 100 L to about 1,000 L, about 100 L to about 500 L, about 100 L to about 250 L, about 100 L to about 200 L, about 100 L to about 150 L, about 150 L to about 15,000 L, about 150 L to about 12,500 L, about 150 L to about 10,000 L, about 150 L to about 8,000 L, about 150 L to about 6,000 L, about 150 L to about 5,000 L, about 150 L to about 4,500 L, about 150 L to about 4,000 L, about 150 L to about 3,500 L, about 150 L to about 3,000 L, about 150 L to about 2,500 L, about 150 L to about 2,000 L, about 150 L to about 1,500 L, about 150 L to about 1,000 L, about 150 L to about 500 L, about 150 L to about 250 L, about 150 L to about 200 L, about 200 L to about 15,000 L, about 200 L to about 12,500 L, about 200 L to about 10,000 L, about 200 L to about 8,000 L, about 200 L to about 6,000 L, about 200 L to about 5,000 L, about 200 L to about 4,500 L, about 200 L to about 4,000 L, about 200 L to about 3,500 L, about 200 L to about 3,000 L, about 200 L to about 2,500 L, about 200 L to about 2,000 L, about 200 L to about 1,500 L, about 200 L to about 1,000 L, about 200 L to about 500 L, about 200 L to about 250 L, about 250 L to about 15,000 L, about 250 L to about 12,500 L, about 250 L to about 10,000 L, about 250 L to about 8,000 L, about 250 L to about 6,000 L, about 250 L to about 5,000 L, about 250 L to about 4,500 L, about 250 L to about 4,000 L, about 250 L to about 3,500 L, about 250 L to about 3,000 L, about 250 L to about 2,500 L, about 250 L to about 2,000 L, about 250 L to about 1,500 L, about 250 L to about 1,000 L, about 250 L to about 500 L, about 500 L to about 15,000 L, about 500 L to about 12,500 L, about 500 L to about 10,000 L, about 500 L to about 8,000 L, about 500 L to about 6,000 L, about 500 L to about 5,000 L, about 500 L to about 4,500 L, about 500 L to about 4,000 L, about 500 L to about 3,500 L, about 500 L to about 3,000 L, about 500 L to about 2,500 L, about 500 L to about 2,000 L, about 500 L to about 1,500 L, about 500 L to about 1,000 L, about 1,000 L to about 15,000 L, about 1,000 L to about 12,500 L, about 1,000 L to about 10,000 L, about 1,000 L to about 8,000 L, about 1,000 L to about 6,000 L, about 1,000 L to about 5,000 L, about 1,000 L to about 4,500 L, about 1,000 L to about 4,000 L, about 1,000 L to about 3,500 L, about 1,000 L to about 3,000 L, about 1,000 L to about 2,500 L, about 1,000 L to about 2,000 L, about 1,000 L to about 1,500 L, about 1,500 L to about 15,000 L, about 1,500 L to about 12,500 L, about 1,500 L to about 10,000 L, about 1,500 L to about 8,000 L, about 1,500 L to about 6,000 L, about 1,500 L to about 5,000 L, about 1,500 L to about 4,500 L, about 1,500 L to about 4,000 L, about 1,500 L to about 3,500 L, about 1,500 L to about 3,000 L, about 1,500 L to about 2,500 L, about 1,500 L to about 2,000 L, about 2,000 L to about 15,000 L, about 2,000 L to about 12,500 L, about 2,000 L to about 10,000 L, about 2,000 L to about 8,000 L, about 2,000 L to about 6,000 L, about 2,000 L to about 5,000 L, about 2,000 L to about 4,500 L, about 2,000 L to about 4,000 L, about 2,000 L to about 3,500 L, about 2,000 L to about 3,000 L, about 2,000 L to about 2,500 L, about 2,500 L to about 15,000 L, about 2,500 L to about 12,500 L, about 2,500 L to about 10,000 L, about 2,500 L to about 8,000 L, about 2,500 L to about 6,000 L, about 2,500 L to about 5,000 L, about 2,500 L to about 4,500 L, about 2,500 L to about 4,000 L, about 2,500 L to about 3,500 L, about 2,500 L to about 3,000 L, about 3,000 L to about 15,000 L, about 3,000 L to about 12,500 L, about 3,000 L to about 10,000 L, about 3,000 L to about 8,000 L, about 3,000 L to about 6,000 L, about 3,000 L to about 5,000 L, about 3,000 L to about 4,500 L, about 3,000 L to about 4,000 L, about 3,000 L to about 3,500 L, about 3,500 L to about 15,000 L, about 3,500 L to about 12,500 L, about 3,500 L to about 10,000 L, about 3,500 L to about 8,000 L, about 3,500 L to about 6,000 L, about 3,500 L to about 5,000 L, about 3,500 L to about 4,500 L, about 3,500 L to about 4,000 L, about 4,000 L to about 15,000 L, about 4,000 L to about 12,500 L, about 4,000 L to about 10,000 L, about 4,000 L to about 8,000 L, about 4,000 L to about 6,000 L, about 4,000 L to about 5,000 L, about 4,000 L to about 4,500 L, about 4,500 L to about 15,000 L, about 4,500 L to about 12,500 L, about 4,500 L to about 10,000 L, about 4,500 L to about 8,000 L, about 4,500 L to about 6,000 L, about 4,500 L to about 5,000 L, about 5,000 L to about 15,000 L, about 5,000 L to about 12,500 L, about 5,000 L to about 10,000 L, about 5,000 L to about 8,000 L, about 5,000 L to about 6,000 L, about 6,000 L to about 15,000 L, about 6,000 L to about 12,500 L, about 6,000 L to about 10,000 L, about 6,000 L to about 8,000 L, about 8,000 L to about 15,000 L, about 8,000 L to about 12,500 L, about 8,000 L to about 10,000 L, about 10,000 L to about 15,000 L, about 10,000 L to about 12,500 L, or about 12,500 L to about 15,000 L).

Agitation

In some embodiments, the fed-batch culturing further includes agitating the cell culture. In some embodiments, agitating the cell culture can include agitating at about 10 RPM to about 500 RPM (e.g., about 10 RPM to about 500 RPM, about 10 RPM to about 450 RPM, about 10 RPM to about 400 RPM, about 10 RPM to about 350 RPM, about 10 RPM to about 300 RPM, about 10 RPM to about 280 RPM, about 10 RPM to about 260 RPM, about 10 RPM to about 240 RPM, about 10 RPM to about 220 RPM, about 10 RPM to about 200 RPM, about 10 RPM to about 180 RPM, about 10 RPM to about 160 RPM, about 10 RPM to about 140 RPM, about 10 RPM to about 120 RPM, about 10 RPM to about 100 RPM, about 10 RPM to about 90 RPM, about 10 RPM to about 80 RPM, about 10 RPM to about 70 RPM, about 10 RPM to about 60 RPM, about 10 RPM to about 55 RPM, about 10 RPM to about 50 RPM, about 10 RPM to about 45 RPM, about 10 RPM to about 40 RPM, about 10 RPM to about 35 RPM, about 10 RPM to about 30 RPM, about 10 RPM to about 25 RPM, about 10 RPM to about 20 RPM, about 10 RPM to about 15 RPM, about 15 RPM to about 500 RPM, about 15 RPM to about 450 RPM, about 15 RPM to about 400 RPM, about 15 RPM to about 350 RPM, about 15 RPM to about 300 RPM, about 15 RPM to about 280 RPM, about 15 RPM to about 260 RPM, about 15 RPM to about 240 RPM, about 15 RPM to about 220 RPM, about 15 RPM to about 200 RPM, about 15 RPM to about 180 RPM, about 15 RPM to about 160 RPM, about 15 RPM to about 140 RPM, about 15 RPM to about 120 RPM, about 15 RPM to about 100 RPM, about 15 RPM to about 90 RPM, about 15 RPM to about 80 RPM, about 15 RPM to about 70 RPM, about 15 RPM to about 60 RPM, about 15 RPM to about 55 RPM, about 15 RPM to about 50 RPM, about 15 RPM to about 45 RPM, about 15 RPM to about 40 RPM, about 15 RPM to about 35 RPM, about 15 RPM to about 30 RPM, about 15 RPM to about 25 RPM, about 15 RPM to about 20 RPM, about 20 RPM to about 500 RPM, about 20 RPM to about 450 RPM, about 20 RPM to about 400 RPM, about 20 RPM to about 350 RPM, about 20 RPM to about 300 RPM, about 20 RPM to about 280 RPM, about 20 RPM to about 260 RPM, about 20 RPM to about 240 RPM, about 20 RPM to about 220 RPM, about 20 RPM to about 200 RPM, about 20 RPM to about 180 RPM, about 20 RPM to about 160 RPM, about 20 RPM to about 140 RPM, about 20 RPM to about 120 RPM, about 20 RPM to about 100 RPM, about 20 RPM to about 90 RPM, about 20 RPM to about 80 RPM, about 20 RPM to about 70 RPM, about 20 RPM to about 60 RPM, about 20 RPM to about 55 RPM, about 20 RPM to about 50 RPM, about 20 RPM to about 45 RPM, about 20 RPM to about 40 RPM, about 20 RPM to about 35 RPM, about 20 RPM to about 30 RPM, about 20 RPM to about 25 RPM, about 25 RPM to about 500 RPM, about 25 RPM to about 450 RPM, about 25 RPM to about 400 RPM, about 25 RPM to about 350 RPM, about 25 RPM to about 300 RPM, about 25 RPM to about 280 RPM, about 25 RPM to about 260 RPM, about 25 RPM to about 240 RPM, about 25 RPM to about 220 RPM, about 25 RPM to about 200 RPM, about 25 RPM to about 180 RPM, about 25 RPM to about 160 RPM, about 25 RPM to about 140 RPM, about 25 RPM to about 120 RPM, about 25 RPM to about 100 RPM, about 25 RPM to about 90 RPM, about 25 RPM to about 80 RPM, about 25 RPM to about 70 RPM, about 25 RPM to about 60 RPM, about 25 RPM to about 55 RPM, about 25 RPM to about 50 RPM, about 25 RPM to about 45 RPM, about 25 RPM to about 40 RPM, about 25 RPM to about 35 RPM, about 25 RPM to about 30 RPM, about 30 RPM to about 500 RPM, about 30 RPM to about 450 RPM, about 30 RPM to about 400 RPM, about 30 RPM to about 350 RPM, about 30 RPM to about 300 RPM, about 30 RPM to about 280 RPM, about 30 RPM to about 260 RPM, about 30 RPM to about 240 RPM, about 30 RPM to about 220 RPM, about 30 RPM to about 200 RPM, about 30 RPM to about 180 RPM, about 30 RPM to about 160 RPM, about 30 RPM to about 140 RPM, about 30 RPM to about 120 RPM, about 30 RPM to about 100 RPM, about 30 RPM to about 90 RPM, about 30 RPM to about 80 RPM, about 30 RPM to about 70 RPM, about 30 RPM to about 60 RPM, about 30 RPM to about 55 RPM, about 30 RPM to about 50 RPM, about 30 RPM to about 45 RPM, about 30 RPM to about 40 RPM, about 30 RPM to about 35 RPM about 35 RPM to about 500 RPM, about 35 RPM to about 450 RPM, about 35 RPM to about 400 RPM, about 35 RPM to about 350 RPM, about 35 RPM to about 300 RPM, about 35 RPM to about 280 RPM, about 35 RPM to about 260 RPM, about 35 RPM to about 240 RPM, about 35 RPM to about 220 RPM, about 35 RPM to about 200 RPM, about 35 RPM to about 180 RPM, about 35 RPM to about 160 RPM, about 35 RPM to about 140 RPM, about 35 RPM to about 120 RPM, about 35 RPM to about 100 RPM, about 35 RPM to about 90 RPM, about 35 RPM to about 80 RPM, about 35 RPM to about 70 RPM, about 35 RPM to about 60 RPM, about 35 RPM to about 55 RPM, about 35 RPM to about 50 RPM, about 35 RPM to about 45 RPM, about 35 RPM to about 40 RPM, about 40 RPM to about 500 RPM, about 40 RPM to about 450 RPM, about 40 RPM to about 400 RPM, about 40 RPM to about 350 RPM, about 40 RPM to about 300 RPM, about 40 RPM to about 280 RPM, about 40 RPM to about 260 RPM, about 40 RPM to about 240 RPM, about 40 RPM to about 220 RPM, about 40 RPM to about 200 RPM, about 40 RPM to about 180 RPM, about 40 RPM to about 160 RPM, about 40 RPM to about 140 RPM, about 40 RPM to about 120 RPM, about 40 RPM to about 100 RPM, about 40 RPM to about 90 RPM, about 40 RPM to about 80 RPM, about 40 RPM to about 70 RPM, about 40 RPM to about 60 RPM, about 40 RPM to about 55 RPM, about 40 RPM to about 50 RPM, about 40 RPM to about 45 RPM, about 45 RPM to about 500 RPM, about 45 RPM to about 450 RPM, about 45 RPM to about 400 RPM, about 45 RPM to about 350 RPM, about 45 RPM to about 300 RPM, about 45 RPM to about 280 RPM, about 45 RPM to about 260 RPM, about 45 RPM to about 240 RPM, about 45 RPM to about 220 RPM, about 45 RPM to about 200 RPM, about 45 RPM to about 180 RPM, about 45 RPM to about 160 RPM, about 45 RPM to about 140 RPM, about 45 RPM to about 120 RPM, about 45 RPM to about 100 RPM, about 45 RPM to about 90 RPM, about 45 RPM to about 80 RPM, about 45 RPM to about 70 RPM, about 45 RPM to about 60 RPM, about 45 RPM to about 55 RPM, about 45 RPM to about 50 RPM, about 50 RPM to about 500 RPM, about 50 RPM to about 450 RPM, about 50 RPM to about 400 RPM, about 50 RPM to about 350 RPM, about 50 RPM to about 300 RPM, about 50 RPM to about 280 RPM, about 50 RPM to about 260 RPM, about 50 RPM to about 240 RPM, about 50 RPM to about 220 RPM, about 50 RPM to about 200 RPM, about 50 RPM to about 180 RPM, about 50 RPM to about 160 RPM, about 50 RPM to about 140 RPM, about 50 RPM to about 120 RPM, about 50 RPM to about 100 RPM, about 50 RPM to about 90 RPM, about 50 RPM to about 80 RPM, about 50 RPM to about 70 RPM, about 50 RPM to about 60 RPM, about 50 RPM to about 55 RPM, about 55 RPM to about 500 RPM, about 55 RPM to about 450 RPM, about 55 RPM to about 400 RPM, about 55 RPM to about 350 RPM, about 55 RPM to about 300 RPM, about 55 RPM to about 280 RPM, about 55 RPM to about 260 RPM, about 55 RPM to about 240 RPM, about 55 RPM to about 220 RPM, about 55 RPM to about 200 RPM, about 55 RPM to about 180 RPM, about 55 RPM to about 160 RPM, about 55 RPM to about 140 RPM, about 55 RPM to about 120 RPM, about 55 RPM to about 100 RPM, about 55 RPM to about 90 RPM, about 55 RPM to about 80 RPM, about 55 RPM to about 70 RPM, about 55 RPM to about 60 RPM, about 60 RPM to about 500 RPM, about 60 RPM to about 450 RPM, about 60 RPM to about 400 RPM, about 60 RPM to about 350 RPM, about 60 RPM to about 300 RPM, about 60 RPM to about 280 RPM, about 60 RPM to about 260 RPM, about 60 RPM to about 240 RPM, about 60 RPM to about 220 RPM, about 60 RPM to about 200 RPM, about 60 RPM to about 180 RPM, about 60 RPM to about 160 RPM, about 60 RPM to about 140 RPM, about 60 RPM to about 120 RPM, about 60 RPM to about 100 RPM, about 60 RPM to about 90 RPM, about 60 RPM to about 80 RPM, about 60 RPM to about 70 RPM, about 70 RPM to about 500 RPM, about 70 RPM to about 450 RPM, about 70 RPM to about 400 RPM, about 70 RPM to about 350 RPM, about 70 RPM to about 300 RPM, about 70 RPM to about 280 RPM, about 70 RPM to about 260 RPM, about 70 RPM to about 240 RPM, about 70 RPM to about 220 RPM, about 70 RPM to about 200 RPM, about 70 RPM to about 180 RPM, about 70 RPM to about 160 RPM, about 70 RPM to about 140 RPM, about 70 RPM to about 120 RPM, about 70 RPM to about 100 RPM, about 70 RPM to about 90 RPM, about 70 RPM to about 80 RPM, about 80 RPM to about 500 RPM, about 80 RPM to about 450 RPM, about 80 RPM to about 400 RPM, about 80 RPM to about 350 RPM, about 80 RPM to about 300 RPM, about 80 RPM to about 280 RPM, about 80 RPM to about 260 RPM, about 80 RPM to about 240 RPM, about 80 RPM to about 220 RPM, about 80 RPM to about 200 RPM, about 80 RPM to about 180 RPM, about 80 RPM to about 160 RPM, about 80 RPM to about 140 RPM, about 80 RPM to about 120 RPM, about 80 RPM to about 100 RPM, about 80 RPM to about 90 RPM, about 90 RPM to about 500 RPM, about 90 RPM to about 450 RPM, about 90 RPM to about 400 RPM, about 90 RPM to about 350 RPM, about 90 RPM to about 300 RPM, about 90 RPM to about 280 RPM, about 90 RPM to about 260 RPM, about 90 RPM to about 240 RPM, about 90 RPM to about 220 RPM, about 90 RPM to about 200 RPM, about 90 RPM to about 180 RPM, about 90 RPM to about 160 RPM, about 90 RPM to about 140 RPM, about 90 RPM to about 120 RPM, about 90 RPM to about 100 RPM, about 100 RPM to about 500 RPM, about 100 RPM to about 450 RPM, about 100 RPM to about 400 RPM, about 100 RPM to about 350 RPM, about 100 RPM to about 300 RPM, about 100 RPM to about 280 RPM, about 100 RPM to about 260 RPM, about 100 RPM to about 240 RPM, about 100 RPM to about 220 RPM, about 100 RPM to about 200 RPM, about 100 RPM to about 180 RPM, about 100 RPM to about 160 RPM, about 100 RPM to about 140 RPM, about 100 RPM to about 120 RPM, about 120 RPM to about 500 RPM, about 120 RPM to about 450 RPM, about 120 RPM to about 400 RPM, about 120 RPM to about 350 RPM, about 120 RPM to about 300 RPM, about 120 RPM to about 280 RPM, about 120 RPM to about 260 RPM, about 120 RPM to about 240 RPM, about 120 RPM to about 220 RPM, about 120 RPM to about 200 RPM, about 120 RPM to about 180 RPM, about 120 RPM to about 160 RPM, about 120 RPM to about 140 RPM, about 140 RPM to about 500 RPM, about 140 RPM to about 450 RPM, about 140 RPM to about 400 RPM, about 140 RPM to about 350 RPM, about 140 RPM to about 300 RPM, about 140 RPM to about 280 RPM, about 140 RPM to about 260 RPM, about 140 RPM to about 240 RPM, about 140 RPM to about 220 RPM, about 140 RPM to about 200 RPM, about 140 RPM to about 180 RPM, about 140 RPM to about 160 RPM, about 160 RPM to about 500 RPM, about 160 RPM to about 450 RPM, about 160 RPM to about 400 RPM, about 160 RPM to about 350 RPM, about 160 RPM to about 300 RPM, about 160 RPM to about 280 RPM, about 160 RPM to about 260 RPM, about 160 RPM to about 240 RPM, about 160 RPM to about 220 RPM, about 160 RPM to about 200 RPM, about 160 RPM to about 180 RPM, about 180 RPM to about 500 RPM, about 180 RPM to about 450 RPM, about 180 RPM to about 400 RPM, about 180 RPM to about 350 RPM, about 180 RPM to about 300 RPM, about 180 RPM to about 280 RPM, about 180 RPM to about 260 RPM, about 180 RPM to about 240 RPM, about 180 RPM to about 220 RPM, about 180 RPM to about 200 RPM, about 200 RPM to about 500 RPM, about 200 RPM to about 450 RPM, about 200 RPM to about 400 RPM, about 200 RPM to about 350 RPM, about 200 RPM to about 300 RPM, about 200 RPM to about 280 RPM, about 200 RPM to about 260 RPM, about 200 RPM to about 240 RPM, about 200 RPM to about 220 RPM, about 220 RPM to about 500 RPM, about 220 RPM to about 450 RPM, about 220 RPM to about 400 RPM, about 220 RPM to about 350 RPM, about 220 RPM to about 300 RPM, about 220 RPM to about 280 RPM, about 220 RPM to about 260 RPM, about 220 RPM to about 240 RPM, about 240 RPM to about 500 RPM, about 240 RPM to about 450 RPM, about 240 RPM to about 400 RPM, about 240 RPM to about 350 RPM, about 240 RPM to about 300 RPM, about 240 RPM to about 280 RPM, about 240 RPM to about 260 RPM, about 260 RPM to about 500 RPM, about 260 RPM to about 450 RPM, about 260 RPM to about 400 RPM, about 260 RPM to about 350 RPM, about 260 RPM to about 300 RPM, about 260 RPM to about 280 RPM, about 280 RPM to about 500 RPM, about 280 RPM to about 450 RPM, about 280 RPM to about 400 RPM, about 280 RPM to about 350 RPM, about 280 RPM to about 300 RPM, about 300 RPM to about 500 RPM, about 300 RPM to about 450 RPM, about 300 RPM to about 400 RPM, about 300 RPM to about 350 RPM, about 350 RPM to about 500 RPM, about 350 RPM to about 450 RPM, about 350 RPM to about 400 RPM, about 400 RPM to about 500 RPM, about 400 RPM to about 450 RPM, about 450 RPM to about 500 RPM). It will be appreciated that the size of the cell culture can influence the choice of RPM for agitation.

In some embodiments, agitating the cell culture can include agitation using an impeller tip speed of about 0.4 m/s to about 4.0 m/s (e.g., about 0.4 m/s to about 3.5 m/s, about 0.4 m/s to about 3.0 m/s, about 0.4 m/s to about 2.5 m/s, about 0.4 m/s to about 2.0 m/s, about 0.4 m/s to about 1.5 m/s, about 0.4 m/s to about 1.0 m/s, about 0.4 m/s to about 0.5 m/s, about 0.5 m/s to about 4.0 m/s, about 0.5 m/s to about 3.5 m/s, about 0.5 m/s to about 3.0 m/s, about 0.5 m/s to about 2.5 m/s, about 0.5 m/s to about 3.0 m/s, about 0.5 m/s to about 2.5 m/s, about 0.5 m/s to about 2.0 m/s, about 0.5 m/s to about 1.5 m/s, about 0.5 m/s to about 1.0 m/s, about 1.0 m/s to about 4.0 m/s, about 1.0 m/s to about 3.5 m/s, about 1.0 m/s to about 3.0 m/s, about 1.0 m/s to about 2.5 m/s, about 1.0 m/s to about 2.0 m/s, about 1.0 m/s to about 1.5 m/s, about 1.5 m/s to about 4.0 m/s, about 1.5 m/s to about 3.5 m/s, about 1.5 m/s to about 3.0 m/s, about 1.5 m/s to about 2.5 m/s, about 1.5 m/s to about 2.0 m/s, about 2.0 m/s to about 4.0 m/s, about 2.0 m/s to about 3.5 m/s, about 2.0 m/s to about 3.0 m/s, about 2.0 m/s to about 2.5 m/s, about 2.5 m/s to about 4.0 m/s, about 2.5 m/s to about 3.5 m/s, about 2.5 m/s to about 3.0 m/s, about 3.0 m/s to about 4.0 m/s, about 3.0 m/s to about 3.5 m/s, or about 3.5 m/s to about 4.0 m/s).

In some embodiments, agitating the cell culture can include agitation using an impeller power consumption per volume (P/V) of about 10 W/m³ to about 35 W/m³ (e.g., about 10 W/m³ to about 30 W/m³, about 10 W/m³ to about 25 W/m³, about 10 W/m³ to about 20 W/m³, about 10 W/m³ to about 15 W/m³, about 15 W/m³ to about 35 W/m³, about 15 W/m³ to about 30 W/m³, about 15 W/m³ to about 25 W/m³, about 15 W/m³ to about 20 W/m³, about 20 W/m³ to about 35 W/m³, about 20 W/m³ to about 30 W/m³, about 20 W/m³ to about 25 W/m³, about 25 W/m³ to about 35 W/m³, about 25 W/m³ to about 30 W/m³, or about 30 W/m³ to about 35 W/m³).

The agitation can be performed using a humidified atmosphere controlled atmosphere (e.g., at a humidity of greater than 20%, 30%, 40%, 50%, 60%, 70%, 75%, 80%, 85%, 90%, or 95%, or a humidity of 100%).

Temperature

The culturing methods described herein can be performed at a temperature of about 31° C. to about 40° C. Skilled practitioners will appreciate that the temperature can be changed at specific time point(s) in the culturing method, e.g., day 6, day 7, day 8, day 9, day 10, or day 11 of the culture. In some embodiments, the temperature of the culture is adjusted from a first temperature of about 35.0° C. to about 38.0° C. (e.g., about 35.0° C. to about 37.5° C., about 35.0° C. to about 37.0° C., about 35.0° C. to about 36.5° C., about 35.0° C. to about 36.0° C., about 35.0° C. to about 35.5° C., about 35.5° C. to about 38.0° C., about 35.5° C. to about 37.5° C., about 35.5° C. to about 37.0° C., about 35.5° C. to about 36.5° C., about 35.5° C. to about 36.0° C., about 36.0° C. to about 38.0° C., about 36.0° C. to about 37.5° C., about 36.0° C. to about 37.0° C., about 36.0° C. to about 36.5° C., about 36.5° C. to about 38.0° C., about 36.5° C. to about 37.5° C., about 36.5° C. to about 37.0° C., about 37.0° C. to about 38.0° C., about 37.0° C. to about 37.5° C., or about 37.5° C. to about 38.0° C.) to a second temperature about 28.0° C. to about 34.9° C. (e.g., about 28.0° C. to about 34.5° C., about 28.0° C. to about 34.0° C., about 28.0° C. to about 33.5° C., about 28.0° C. to about 33.0° C., about 28.0° C. to about 32.5° C., about 28.0° C. to about 32.0° C., about 28.0° C. to about 31.5° C., about 28.0° C. to about 31.0° C., about 28.0° C. to about 30.5° C., about 28.0° C. to about 30.0° C., about 28.0° C. to about 29.5° C., about 28.0° C. to about 29.0° C., about 28.0° C. to about 28.5° C., about 28.5° C. to about 34.9° C., about 28.5° C. to about 34.5° C., about 28.5° C. to about 34.0° C., about 28.5° C. to about 33.5° C., about 28.5° C. to about 33.0° C., about 28.5° C. to about 32.5° C., about 28.5° C. to about 32.0° C., about 28.5° C. to about 31.5° C., about 28.5° C. to about 31.0° C., about 28.5° C. to about 30.5° C., about 28.5° C. to about 30.0° C., about 28.5° C. to about 29.5° C., about 28.5° C. to about 29.0° C., about 29.0° C. to about 34.9° C., about 29.0° C. to about 34.5° C., about 29.0° C. to about 34.0° C., about 29.0° C. to about 33.5° C., about 29.0° C. to about 33.0° C., about 29.0° C. to about 32.5° C., about 29.0° C. to about 32.0° C., about 29.0° C. to about 31.5° C., about 29.0° C. to about 31.0° C., about 29.0° C. to about 30.5° C., about 29.0° C. to about 30.0° C., about 29.0° C. to about 29.5° C., about 29.5° C. to about 34.9° C., about 29.5° C. to about 34.5° C., about 29.5° C. to about 34.0° C., about 29.5° C. to about 33.5° C., about 29.5° C. to about 33.0° C., about 29.5° C. to about 32.5° C., about 29.5° C. to about 32.0° C., about 29.5° C. to about 31.5° C., about 29.5° C. to about 31.0° C., about 29.5° C. to about 30.5° C., about 29.5° C. to about 30.0° C., about 30.0° C. to about 34.9° C., about 30.0° C. to about 34.5° C., about 30.0° C. to about 34.0° C., about 30.0° C. to about 33.5° C., about 30.0° C. to about 33.0° C., about 30.0° C. to about 32.5° C., about 30.0° C. to about 32.0° C., about 30.0° C. to about 31.5° C., about 30.0° C. to about 31.0° C., about 30.0° C. to about 30.5° C., about 30.5° C. to about 34.9° C., about 30.5° C. to about 34.5° C., about 30.5° C. to about 34.0° C., about 30.5° C. to about 33.5° C., about 30.5° C. to about 33.0° C., about 30.5° C. to about 32.5° C., about 30.5° C. to about 32.0° C., about 30.5° C. to about 31.5° C., about 30.5° C. to about 31.0° C., about 31.0° C. to about 34.9° C., about 31.0° C. to about 34.5° C., about 31.0° C. to about 34.0° C., about 31.0° C. to about 33.5° C., about 31.0° C. to about 33.0° C., about 31.0° C. to about 32.5° C., about 31.0° C. to about 32.0° C., about 31.0° C. to about 31.5° C., about 31.5° C. to about 34.9° C., about 31.5° C. to about 34.5° C., about 31.5° C. to about 34.0° C., about 31.5° C. to about 33.5° C., about 31.5° C. to about 33.0° C., about 31.5° C. to about 32.5° C., about 31.5° C. to about 32.0° C., about 32.0° C. to about 34.9° C., about 32.0° C. to about 34.5° C., about 32.0° C. to about 34.0° C., about 32.0° C. to about 33.5° C., about 32.0° C. to about 33.0° C., about 32.0° C. to about 32.5° C., about 32.5° C. to about 34.9° C., about 32.5° C. to about 34.5° C., about 32.5° C. to about 34.0° C., about 32.5° C. to about 33.5° C., about 32.5° C. to about 33.0° C., about 33.0° C. to about 34.9° C., about 33.0° C. to about 34.5° C., about 33.0° C. to about 34.0° C., about 33.0° C. to about 33.5° C., about 33.5° C. to about 34.9° C., about 33.5° C. to about 34.5° C., about 33.5° C. to about 34.0° C., about 34.0° C. to about 34.9° C., about 34.0° C. to about 34.5° C., or about 34.5° C. to about 34.9° C.) at, e.g., day 6, day 7, day 8, day 9, day 10, or day 11 of the culture.

pH

In some embodiments, the fed-batch culturing further includes maintaining the pH of the cell culture at about 6.5 to about 7.3 (e.g., about 6.5 to about 7.2, about 6.5 to about 7.1, about 6.5 to about 7.0, about 6.5 to about 6.9, about 6.5 to about 6.8, about 6.5 to about 6.7, about 6.5 to about 6.6, about 6.6 to about 7.3, about 6.6 to about 7.2, about 6.6 to about 7.1, about 6.6 to about 7.0, about 6.6 to about 6.9, about 6.6 to about 6.8, about 6.6 to about 6.7, about 6.7 to about 7.3, about 6.7 to about 7.2, about 6.7 to about 7.1 about 6.7 to about 7.0, about 6.7 to about 6.9, about 6.7 to about 6.8, about 6.8 to about 7.3, about 6.8 to about 7.2, about 6.8 to about 7.1, about 6.8 to about 7.0, about 6.8 to about 6.9, about 6.9 to about 7.3, about 6.9 to about 7.2, about 6.9 to about 7.1, about 6.9 to about 7.0, about 7.0 to about 7.3, about 7.0 to about 7.2, about 7.0 to about 7.1, about 7.1 to about 7.3, about 7.1 to about 7.2, or about 7.2 to about 7.3). In some embodiments, the fed-batch culturing further includes maintaining the pH at about 6.80 to about 7.00 (e.g., about 6.80 to about 6.98, about 6.80 to about 6.96, about 6.80 to about 6.94, about 6.80 to about 6.92, about 6.80 to about 6.90, about 6.80 to about 6.88, about 6.80 to about 6.86, about 6.80 to about 6.84, about 6.80 to about 6.82, about 6.82 to about 7.00, about 6.82 to about 6.98, about 6.82 to about 6.96, about 6.82 to about 6.94, about 6.82 to about 6.92, about 6.82 to about 6.90, about 6.82 to about 6.88, about 6.82 to about 6.86, about 6.82 to about 6.84, about 6.84 to about 7.00, about 6.84 to about 6.98, about 6.84 to about 6.96, about 6.84 to about 6.94, about 6.84 to about 6.92, about 6.84 to about 6.90, about 6.84 to about 6.88, about 6.84 to about 6.86, about 6.86 to about 7.00, about 6.86 to about 6.98, about 6.86 to about 6.96, about 6.86 to about 6.94, about 6.86 to about 6.92, about 6.86 to about 6.90, about 6.86 to about 6.88, about 6.88 to about 7.00, about 6.88 to about 6.98, about 6.88 to about 6.96, about 6.88 to about 6.94, about 6.88 to about 6.92, about 6.88 to about 6.90, about 6.90 to about 7.00, about 6.90 to about 6.98, about 6.90 to about 6.96, about 6.90 to about 6.94, about 6.90 to about 6.92, about 6.92 to about 7.00, about 6.92 to about 6.98, about 6.92 to about 6.96, about 6.92 to about 6.94, about 6.94 to about 7.00, about 6.94 to about 6.98, about 6.94 to about 6.96, about 6.96 to about 7.00, about 6.96 to about 6.98, or about 6.98 to about 7.00).

Feed Medium Addition

The first feed medium and the second feed medium can be added to the liquid culture medium, e.g., by perfusion pump. The first feed medium and the second feed medium can be added to the liquid culture medium manually (e.g., by pipetting the first feed medium directly onto the liquid culture medium) or in an automated fashion.

CO₂

The methods described herein can further include incubating the cell culture in an atmosphere containing at most or about 15% CO₂ (e.g., at most or about 14% CO₂, 12% CO₂, 10% CO₂, 8% CO₂, 6% CO₂, 5% CO₂, 4% CO₂, 3% CO₂, 2% CO₂, or at most or about 1% CO₂). Moreover, any of the methods described herein can include incubating the cell culture in a humidified atmosphere (e.g., at least or about 20%, 30%, 40%, 50%, 60%, 70%, 85%, 80%, 85%, 90%, or at least or about 95% humidity, or about 100% humidity).

dO₂

The methods described herein the fed-batch culturing further includes maintaining the dO₂ of the cell culture at about 35% to about 45% (e.g., about 35% to about 44%, about 35% to about 43%, about 35% to about 42%, about 35% to about 41%, about 35% to about 40%, about 35% to about 39%, about 35% to about 38%, about 35% to about 37%, about 35% to about 36%, about 36% to about 45%, about 36% to about 44%, about 36% to about 43%, about 36% to about 42%, about 36% to about 41%, about 36% to about 40%, about 36% to about 39%, about 36% to about 38%, about 36% to about 37%, about 37% to about 45%, about 37% to about 44%, about 37% to about 43%, about 37% to about 42%, about 37% to about 41%, about 37% to about 40%, about 37% to about 39%, about 37% to about 38%, about 38% to about 45%, about 38% to about 44%, about 38% to about 43%, about 38% to about 42%, about 38% to about 41%, about 38% to about 40%, about 38% to about 39%, about 39% to about 45%, about 39% to about 44%, about 39% to about 43%, about 39% to about 42%, about 39% to about 41%, about 39% to about 40%, about 40% to about 45%, about 40% to about 44%, about 40% to about 43%, about 40% to about 42%, about 40% to about 41%, about 41% to about 45%, about 41% to about 44%, about 41% to about 43%, about 41% to about 42%, about 42% to about 45%, about 42% to about 44%, about 42% to about 43%, about 43% to about 45%, about 43% to about 44%, or about 44% to about 45%).

### Protein Recovery

The methods described herein can further comprise recovering a recombinant protein from the cell culture. In some embodiments, a recombinant protein can be recovered from the cell culture after about 10 days to about 18 days of culture (e.g. days, about 10 days to about 11 days, about 10 days to about 12 days, about 10 days to about 13 days, about 10 days to about 14 days, about 10 days to about 15 days, about 10 days to about 16 days, about 10 days to about 17 days, about 11 days to about 12 days, about 11 days to about 13 days, about 11 days to about 14 days, about 11 days to about 15 days, about 11 days to about 16 days, about 11 days to about 18 days, about 12 days to about 13 days, about 12 days to about 14 days, about 12 days to about 15 days, about 12 days to about 16 days, about 12 days to about 17 days, about 12 days to about 18 days, about 13 days to about 14 days, about 13 days to about 15 days, about 13 days to about 16 days, about 13 days to about 17 days, about 13 days to about 18 days, about 14 days to about 15 days, about 14 days to about 16 days, about 14 days to about 17 days, about 14 days to about 18 days, about 15 days to about 16 days, about 15 days to about 17 days, about 15 days to about 18 days, about 16 days to about 17 days, about 16 days to about 18 days, about 17 days to about 18 days, about 10 days, about 11 days, about 12 days, about 13 days, about 14 days, about 15 days, about 16 days, about 17 days, or about 18 days). In some embodiments, a recombinant protein can be recovered from the cell culture when the cell viability of the cell culture falls below about 80% (e.g., below about 75%, 70%, 65%, 60%, 55%, 50%, 45%, 40%, 35%, 30%, 25%, or 20%).

### Mammalian Cells Including a Nucleic Acid the Encodes a Recombinant Protein

A nucleic acid encoding a recombinant protein can be introduced into a mammalian cell using a wide variety of methods known in molecular biology and molecular genetics. Non-limiting examples include transfection (e.g., lipofection), transduction (e.g., lentivirus, adenovirus, or retrovirus infection), and electroporation. In some instances, the nucleic acid that encodes a recombinant protein is not stably integrated into a chromosome of the mammalian cell (transient transfection), while in others the nucleic acid is integrated. Alternatively or in addition, the nucleic acid encoding a recombinant protein can be present in a plasmid and/or in a mammalian artificial chromosome (e.g., a human artificial chromosome). Alternatively or in addition, the nucleic acid can be introduced into the cell using a viral vector (e.g., a lentivirus, retrovirus, or adenovirus vector). The nucleic acid can be operably linked to a promoter sequence (e.g., a strong promoter, such as a β-actin promoter and CMV promoter, or an inducible promoter). A vector containing the nucleic acid can, if desired, also contain a selectable marker (e.g., a gene that confers hygromycin, puromycin, or neomycin resistance to the mammalian cell).

In some instances, the recombinant protein is a secreted protein and is released by the mammalian cell into the extracellular medium (e.g., the first and/or second liquid culture medium). For example, a nucleic acid sequence encoding a soluble recombinant protein can contain a sequence that encodes a secretion signal peptide at the N- or C-terminus of the recombinant protein, which is cleaved by an enzyme present in the mammalian cell, and subsequently released into the extracellular medium (e.g., the first and/or second liquid culture medium). In other instances, the recombinant protein is a soluble protein that is not secreted, and the recombinant protein is recovered from within the mammalian cell.

Non-limiting examples of recombinant proteins that can be produced by the methods provided herein include fusion proteins, antibodies, and antibody fragments.

A secreted, soluble recombinant protein can be recovered from the liquid culture medium (e.g., the first and/or second liquid culture medium) by removing or otherwise physically separating the liquid culture medium from the mammalian cells. A variety of different methods for removing liquid culture medium from mammalian cells are known in the art, including, for example, centrifugation, filtration, pipetting, and/or aspiration. The secreted recombinant protein can then be recovered and further purified from the liquid culture medium using a variety of biochemical techniques including various types of chromatography (e.g., affinity chromatography, molecular sieve chromatography, cation exchange chromatography, or anion exchange chromatography) and/or filtration (e.g., molecular weight cut-off filtration).

To recover an intracellular recombinant protein, the mammalian cell can be lysed. A wide variety of methods for lysing mammalian cells are known in the art, including, for example, sonication and/or detergent, enzymatic, and/or chemical lysis. A recombinant protein can be purified from a mammalian cell lysate using a variety of biochemical methods known in the art, typically starting with a step of centrifugation to remove the cellular debris, and then one or more additional steps (e.g., one or more types of chromatography (e.g., affinity chromatography, molecular sieve chromatography, cation exchange chromatography, or anion exchange chromatography) and/or filtration (e.g., molecular weight cut-off filtration)).

In some embodiments, the recovered recombinant protein is at least or about 50% pure by weight, e.g., at least or about 55% pure by weight, at least 60% pure by weight, at least 65% pure by weight, at least 70% pure by weight, at least 75% pure by weight, at least 80% pure by weight, at least 85% pure by weight, at least 90% pure by weight, at least 95% pure by weight, at least 96% pure by weight, at least 97% pure by weight, at least 98% pure by weight, or at least or about 99% pure by weight, or greater than 99% pure by weight.

In some embodiments, the recovering in step (c) occurs on day 14 of the culture. Some embodiments of any of the methods described herein can further include formulating the purified recombinant protein into a pharmaceutical composition.

Also provided are recombinant proteins produced by any of the methods described herein. Also provided are pharmaceutical compositions produced by any of the methods described herein.

Also provided are methods of treating a subject in need thereof that include administering to the subject a therapeutically effective amount of any of the recombinant proteins produced using any of the methods described herein or any of the pharmaceutical compositions produced using any of the methods described herein.

### ADDITIONAL EXEMPLARY ASPECTS

In some embodiments, the method further include generating the cell culture of step (a) by inoculating the liquid culture medium with a population of CHO cells. In some examples, the population of the CHO cells has not been previously cultured in the liquid culture medium.

### EXAMPLES

The invention is further described in the following examples, which do not limit the scope of the invention described in the claims.

### Example 1—Methodology Overview

A goal was to conduct baseline experiments in order to replicate and obtain the baseline data for fed-batch experiments. Generally, fed-batch experiments were initiated from thawing of a monoclonal antibody (e.g. adalimumab) cell line followed by seed train expansion of the inoculum using shake flasks (30 mL, 60 mL, 125 mL, or 250 mL). Typically, two consecutive passages were performed every three days before the official start of the 14-day fed-batch run following standard parameters. In addition, adaptation experiments were initiated from thawing of monoclonal antibody cell line to culturing of cells into shake flasks until the doubling time was consistent throughout three consecutive passages in a batch process. Throughout the duration of process development studies, data were consistently monitored using a Beckmann Coulter Vi-Cell™ Analyzer, a Nova BIOPROFILE® 400, and a freezing-point depression osmometer. Data was generated and analyzed to find the optimal feeding strategies and conditions. Some conditions were eventually scaled up to bench-scale bioreactor experiments to further understand cell growth performance and product quality. Similarly, throughout bench-scale bioreactor experiments, data were generated and analyzed while bioreactor samples were collected for downstream titer and product quality studies.

Criteria evaluated include characteristics of the cells (e.g. viable cell density, cell viability, cell diameter, cell circumference), characteristics of the culture environment (e.g. pH, pO₂, dO₂, CO₂, osmolality, energy source (e.g. glucose), glutamate, glutamine, lactate, ammonium, sodium, potassium), and product characteristics (e.g. titer, product quality (e.g. proper folding, aggregates, fragments, glycoprofile, and activity).

### Example 2—Shake Flask Conditions

Small-scale process development studies were performed using CORNING® non-baffled shake flasks. The initial working volume (wv) was set at 50 mL using CD-C4 media for fed-batch studies and EX-CELL® and HyClone™ ActiPro™ for adaptation experiments. Feed varied throughout study arms testing for different conditions and feeding strategies. N-Acetyl-D-glucosamine was added on days 0 and 6 at a volume of 1.05% and 1.55% of working volume, respectively. In addition, shake flasks were incubated at a temperature of 37° C. and agitated at 130 RPM at 5% CO₂ level.

### Example 3—Bioreactor Conditions

Bioreactor experiments were performed using CD-C4 media followed by inoculation of cell culture derived from the optimal conditions investigated during shake flask studies. Therefore, the type of feed was different throughout the bioreactor study arms. All bioreactor experiments were performed following standard internal experimental parameters and set-points. This included addition of N-Acetyl-D-glucosamine on days 0 and 6 at 1.05% and 1.55% of working volume, respectively. Dissolved oxygen (DO) was maintained at 40%±10% along with agitation of 200 RPM throughout the duration of the experiment. The pH was maintained at 6.9±0.2 on days 0 through 4, followed by a pH set-point and control deadband shift at 6.88±0.02 on days 4 until harvest. Temperature was closely maintained at 36.5° C. from days 0 to 8, followed by a shift at 34° C. from days 8 until harvest.

### Example 4—Monoclonal Antibody Baseline Process

Monoclonal antibody (adalimumab) research cell bank cryovials (Passage 2, day 3, 93% viability, 3×10⁷ cells/mL) were used. These cryovials were derived from a monoclonal antibody (adalimumab) working cell bank. The present data was generated from several control conditions used for experiments performed in shake flasks (n=24) and bioreactor (n=2) runs. Collectively, this data was compiled to assess the baseline process characteristics.

In shake flask control conditions, peak VCD (viable cell density; 5.69×10⁶ cells/ml) was reached at day 7, and cell viability began to drop below 90% until harvest, where a 43% cell viability was observed. The presence of glutamine diminished as the culture progressed. Glucose concentrations increased in the culture, 4.5 mmol/L to 1.5 mmol/L and 5.8 g/L to 10.7 g/L, respectively. Byproduct accumulation was observed for ammonium and lactate, increasing from 0.85 mmol/L to 2.5 mmol/L and 0.47 g/L to 1.15 g/L, respectively. Among the culture conditions, osmolality (300-350 mOsmo) remained relatively constant, while pH decreased as the culture continued, with an initial pH of 7.5 to 6.84 at harvest. With a greater control on culture conditions, bioreactor runs generally displayed superior process characteristics.

In the baseline bioreactor runs, a peak VCD (10.74×10⁶ cells/mL) was reached at day 9, followed by cell viability beginning to decline below 90% by day 11. Like shake flask conditions, glutamine gradually decreased (4.5 to 2 mmol/L) and glucose gradually increased (5.8 to 6.57 g/L). The accumulation of ammonium (1.25 to 3.59 mmol/L) and lactate (0.9 to 1.5 g/L) byproducts were observed throughout the 14-day culture period. Culture conditions were similar to that of shake flasks; however, pH remained controlled according to centerpoint conditions.

When comparing shake flask and bioreactor baseline control runs, shake flasks typically demonstrated a faster doubling time, while bioreactor runs observed a greater IVCD (integrated viable cell density) and peak VCD. The differences observed between conditions can be attributed, in some cases, to a tighter control of culture conditions within a bioreactor. The following Examples compare control conditions that were run in parallel to experimental conditions, evaluating process and product characteristics of several feeding strategies and medium adaptations.

### Feed Investigation

The following Examples investigated potential feeds and feeding strategies that could potentially improve a monoclonal antibody (e.g. adalimumab) fed-batch process. From the results of this feed investigation phase, lab scale bioreactor runs were conducted as a performance assessment. The feed investigation studies included several feeds and feeding strategies. Each experimental condition was run in triplicate and conducted in 250 mL CORNING® non-baffled shake flasks (50 mL working volume (wv)). The following Examples will provide a table of parameters and relevant results from each experiment, followed by a brief discussion of results.

### Example 5—Altered BalanCD® CHO Feed 1 Study

An experiment utilizing BalanCD® CHO Feed 1 with an increased carbon source availability was performed. In preliminary studies, the carbon sources glucose and glutamine were found to be limiting factors in prolonging the growth profiles and viable cell densities.

### Methodology/Experimental Design

This study was conducted to evaluate an altered concentration of the BalanCD® CHO Feed 1 with the established feeding strategy (Table 1). The concentration of glutamine and glucose was doubled generating a 2× Gln & Gluc Feed 1 (“2× Feed 1”) for the experimental condition with the baseline BalanCD® CHO Feed 1 (“CHO Feed 1”) functioning as the control. The feeding strategy remained constant with a 5% working volume feed on days 3 through 8 and 7% working volume feed on days 9 through 13 (Table 1). The initial medium utilized for both conditions was CD-C4 with a working volume of 50 ml in 250 ml shake flasks, performed in triplicate. The standard fed-batch process was conducted over a 14-day period.

TABLE 1
Experimental conditions for altered Feed 1 experiment utilizing a 2X
glucose & glutamine BalanCD ® CHO Feed 1 solution.

| Condition | CHO Feed 1 | 2X Feed 1 |
|---|---|---|
| Feed Type | Feed 1 | 2X Feed 1 |
| Volume (wv) % | 1) 5% | 1) 5% |
| | 2) 7% | 2) 7% |
| Schedule (days) | 1) 3-7 | 1) 3-7 |
| | 2) 8-13 | 2) 8-13 |

### Results & Discussion

Triplicates of shake flasks for both conditions were sampled and fed accordingly to experiment completion on day 14. The VCD values were utilized to generate the IVCD values and the doubling times depicted in Table 2. The doubling times for CHO Feed 1 and the 2× Feed 1 were 32.1 hrs and 35.5 hours respectively. The IVCD for the CHO Feed 1 and the experimental 2× Feed 1 were 70.67 and 57.93 respectively.

TABLE 2
Growth characteristics for altered BalanCD ® Feed 1 conditions.
Despite an increase in concentrations of glucose and glutamine,
baseline conditions outperformed the experimental condition.

| Condition | CHO Feed 1 | 2X Feed 1 |
|---|---|---|
| Peak VCD (×10⁶ | 8.07 | 7.38 |
| cells/mL) | | |
| IVCD | 70.67 | 57.93 |
| (×10⁶ cells × hr/mL) | | |
| Doubling Time (hours) | 32.1 | 35.5 |

This Example was used to observe the growth profile of the adalimumab cell line in a BalanCD® CHO Feed 1 with an increased 2× concentration of glutamine and glucose in comparison to the baseline CHO Feed 1 shake following the same feeding regimen. Overall, the 2× Feed 1 condition did not significantly alter the growth profile of the cell line; on day 6, the growth profile of the 2× Feed 1 condition began to plateau, then decrease while the CHO Feed 1 condition reached a higher VCD. This is further supported by the higher integral viable cell density generated by the CHO Feed 1 shake flasks in comparison to the 2× Feed 1 condition as well as the shorter doubling time exhibited by the CHO Feed 1 as shown in Table 2. According to the average specific net growth profiles, the 2× Feed 1 growth profile did not achieve the growth of adalimumab exhibited in CHO Feed 1, potentially demonstrating that the increased carbon source does not improve the growth profile of the adalimumab cell line. Waste products (e.g., lactate, glutamine, ammonium) were generated in similar trends in the CHO Feed 1 and 2× Feed 1 conditions, demonstrating the similar growth profile of the 2× Feed 1 in comparison to the CHO Feed 1 shake flasks. The stationary phase and the plateau of the growth in VCD correlates with the depletion of glutamine, indicating glutamine to be a possible limiting factor for prolonged growth of the adalimumab cell line (investigated further in Example 6). These results indicate that a 2× increase in concentration of glutamine does not demonstrate an improvement on the viable cell density of the adalimumab cell line. The increased glucose profile exhibited by 2× Feed 1 potentially correlates with the increase in osmolality due to the increase concentration of ions and particles in the medium which subsequently resulted in the lower peak density in the 2× Feed 1 shake flasks.

The assessment of the 2× Feed 1 experimental condition generated results that do not surpass that of the established CHO Feed 1 control.

### Example 6—Feed as Needed (FAN)

To determine if the regulation of glucose and glutamine levels within the cell culture can improve cell growth characteristics, 32 mmol/L glutamine and 32 g/L glucose in CDC4 media was fed separately as needed to the adalimumab cell line to maintain glutamine and glucose levels above 3 mmol/L and 3 g/L respectively.

### Methodology/Experimental Design

For this experiment, there were three conditions in base medium of CD-C4. For the first condition, the control, a standard feeding strategy was used with BalanCD® CHO Feed 1 (“SF CHO F1”). For the second condition, a standard feeding strategy was used with BalanCD® CHO Feed 2. For the third condition, a stock solution of 32 mmol/L glutamine and 32 g/L glucose in CD-C4 medium were added separately to maintain glutamine and glucose levels above 3 mmol/L and 3 g/L, respectively, at all times throughout the culture duration. In each condition, GlcNAc was supplemented to each of the flasks on Days 0 and 6 of approximately 1% of the working volume.

TABLE 3
Experimental conditions for the feed as needed experiment,
utilizing feed 1 strategy and feed 2 as baselines. The
experimental condition (FAN) was maintained at 2 g/L
and 2 mmol/L of glucose and glutamine, respectively.

| Condition | SF CHO F1 | SF CHO F2 | SF FAN |
|---|---|---|---|
| Feed Type | Feed 1 | Feed 2 | 32 g/L glucose and |
| | | | 32 mmol/L glutamine |
| Volume (wv) % | 1) 5% | 1) 5% | Variable |
| | 2) 7% | 2) 7% | |
| Schedule (days) | 1) 3-7 | 1) 3-7 | Variable |
| | 2) 8-13 | 2) 8-13 | |

### Results & Discussion

In the shake flask control condition (SF CHO F1), peak VCD (3.87×10⁶ cells/mL) was reached at day 7 along with cell viability beginning to drop below 90% following Day 6. Glucose concentrations accumulated in the culture following day 3, whereas glutamine diminished and plateaued at around 2 mmol/L following day 8. Byproduct accumulation was observed for lactate and ammonium, increasing from 0.73 g/L to 2.27 g/L and 0.6 mmol/L to 4.81 mmol/L, respectively. Among the culture conditions, osmolality (291-359 mOsmo) remained relatively constant while pH decreased as the culture continued, with an initial pH of 7.6 to 6.82 at harvest. The next condition (SF CHO F2) had similar process characteristics.

In the second affirmatory shake flask condition (SF CHO F2), a peak VCD (4.29×10⁶ cells/mL) was reached at day 8, followed by cell viability beginning to decline below 90% by day 6. Like the SF CHO F1 conditions, glucose gradually increased (6.16 to 9.92 g/L) and glutamine gradually decreased (4.65 to 2.03 mmol/L). The accumulation of lactate (0.73 to 1.22 g/L) and ammonium (0.58 to 2.24 mmol/L) by products were observed throughout the 14-day culture period. Osmolality remained relatively constant between 295 and 340 mOsmo, while pH decreased from 7.6 to 6.8.

In the experimental shake flask condition (SF FAN), a peak VCD (4.02×10⁶ cells/mL) was reached at day 8, followed by cell viability beginning to decline below 90% by day 6. Glucose and glutamine can be seen to be maintained above 3 g/L and 3 mmol/L respectively with variable feeding amounts and times. The accumulation of lactate (0.78 to 1.66 g/L) and ammonium (0.6 to 9.17 mmol/L) was observed. Osmolality remained relatively constant between 295 and 355 mOsmo, while pH decreased from 7.6 to 6.82.

When comparing these conditions, SF CHO F2 (BalanCD® CHO Feed 2) observed a faster doubling time, greater IVCD, and peak VCD (Table 4). It was observed that the ammonium concentration of the SF FAN condition tends be significantly higher than the other conditions from day 3 onwards. Given this, alternative feeds are investigated to be able to supply nutrients more effectively without producing large amounts of detrimental byproducts.

TABLE 4
Feed as Needed (FAN) Study Peak VCD, IVCD, and Doubling
Times in SF CHO F1 (n = 3), SF CHO F2 (n =
3), and SF FAN (n = 3). Among these conditions,
Feed 2 demonstrated favorable growth characteristics
compared to the baseline Feed 1 conditior and experimental
FAN condition.

| Condition | SF CHO F1 | SF CHO F2 | SF FAN |
|---|---|---|---|
| Peak VCD (×10⁶ | 3.87 | 4.29 | 4.02 |
| cells/mL) | | | |
| IVCD | 28.90 | 34.29 | 28.40 |
| (×10⁶ cells × hr/mL) | | | |
| Doubling Time | 31.41 | 31.10 | 40.50 |
| (hours) | | | |

### Example 7—Efficient B Studies

Another feed that was studied was CHO CD Efficient Feed™ B Liquid Nutrient Supplement. Chemically-defined and animal origin-free, the supplement is made by Gibco® and sold by Thermo Fisher Scientific and has no hydrolysates, proteins, or components of incompletely defined composition.

### Methodology/Experimental Design

For this 12-day experiment, there were four conditions with different feeding strategies, all with a base medium of CD-C4. For the first condition, which is the control, a standard feeding strategy was used with BalanCD® CHO Feed 1 (“CHO Feed1”). For the second condition, Efficient Feed B was fed at the same interval and percentage as the control (“CHO EffB”). For the CHO Feed1 and CHO EffB conditions, GlcNAc was supplemented to each of the flasks on Days 0 and 6 of approximately 1% of the working volume. For the third condition and fourth condition, feeding strategies were determined by manufacturer recommendation. In the third condition, Efficient Feed B was fed at 10% of the working volume on Days 2, 4, 6, 8, 10, and 12 (“E2D EffB”). Finally, for the fourth condition, Efficient Feed B was fed at 10% of the working volume on Days 3, 6, 9, and 12 (“E3D EffB”) (Table 5).

TABLE 5
Experimental conditions for an Efficient B study utilizing a
standard feeding strategy and recommended feeding strategies.

| Condition | CHO_Feed1 | CHO_EffB | E2D_EffB | E3D_EffB |
|---|---|---|---|---|
| Feed Type | Feed 1 | Efficient B | Efficient B | Efficient B |
| Volume | 1) 5% | 1) 5% | 10% | 10% |
| (wv) % | 2) 7% | 2) 7% | | |
| Schedule | 1) 3-7 | 1) 3-7 | 2, 4, 6, | 3, 6, 9, 12 |
| (days) | 2) 8-13 | 2) 8-13 | 8, 10, 12 | |

### Results & Discussion

In all conditions, a peak VCD was reached between about day 7 and about day 9 coinciding with a drop in cell viability below 90%. While three conditions achieved about a peak VCD of 5.5×10⁶ cells/mL, the E3D condition had a higher VCD after day 7 with a peak VCD of 6.19×10⁶ cells/mL. Glucose concentrations increased in the culture for the first two conditions (CHO Feed 1 and CHO EffB), whereas the glutamine concentration diminished as the culture progressed for the first two conditions with an average of 5.8 g/L to 8.0 g/L and 4.8 mmol/L to 1.0 mmol/L, respectively. For the third (E2D EffB) condition, there was a much higher and fluctuating glucose trend, whereas the fourth (E3D EffB) condition had a lower and fluctuating glucose trend. Like all the other conditions, glutamine for the E3D EffB condition gradually decreased; however, it was slightly lower after day 7. For each of these conditions, byproduct accumulation was observed for lactate and ammonium, increasing from about 0.9 g/L to 1.2 g/L and 0.7 mmol/L to 4.3 mmol/L, respectively. Also, for each of these conditions, osmolality (390-325 mOsmo) remained relatively constant while pH decreased as the culture continued, from 7.6 to 6.7.

When comparing these conditions, E3D EffB observed a faster doubling time, greater IVCD, and peak VCD (Table 6). It was observed that the glutamine concentration of the E3D condition tended be lower than the other conditions from day 7 onwards. Given this, Example 8 was performed to determine if doubling the glutamine concentration in Efficient Feed B could yield in better IVCD values and doubling times.

TABLE 6
Efficient B Study Peak VCD, IVCD, and Doubling Times in
CHO Feed 1 (n = 3), CHO EffB (n = 3), E2D EffB
(n = 3) and E3D EffB (n = 3). E3D conditions
produced favorable growth characteristics, outperforming
the other conditions in all 3 growth measurements.

| Condition | CHO_Feed1 | E3D_EffB | CHO_EffB | E2D_EffB |
|---|---|---|---|---|
| Peak VCD | 5.46 | 6.19 | 5.59 | 5.51 |
| (×10⁶ | | | | |
| cells/mL) | | | | |
| IVCD | 43.92 | 45.76 | 41.71 | 41.62 |
| (×10⁶ | | | | |
| cells × hr/mL) | | | | |
| Doubling Time | 33.0 | 31.4 | 33.4 | 33.2 |
| (hours) | | | | |

### Example 8—Further Efficient B Studies

Based on the results of Example 7, another study was conducted for a total of 9 days to assess if doubling the glutamine concentration in the feed would yield better results. For this experiment, there were two experimental conditions, both of which were fed at a 10% working volume every third day. The first experimental condition was fed with Efficient Feed B (“E3D EffB”), and the second experimental condition was fed with Efficient Feed B containing double the glutamine concentration (“E3D EffB2×Gln”).

TABLE 7
Experimental conditions for an Efficient B study further evaluating
every 3rd day feeding in addition to an Efficient Feed B
condition containing 2x glutamine concentrations.

| Condition | CHO_Feed1 | E3D_EffB | E3D_EffB2xGln |
|---|---|---|---|
| Feed Type | Feed 1 | Efficient B | Efficient B - 2X |
| | | | Glutamine |
| | | | Concentration |
| Volume (wv) % | 1) 5% | 10% | 10% |
| | 2) 7% | | |
| Schedule (days) | 1) 3-7 | 3, 6, 9 | 3, 6, 9 |
| | 2) 8-13 | | |

### Results & Discussion

For a better comparison, the two experimental conditions were also compared with the control (CHO Feed 1) from the previous Example. In the normal E3D Efficient B condition, a peak VCD (10.55×10⁶ cells/mL) was reached at day 8, followed by cell viability beginning to decline below 90% on the same day. In the double glutamine E3D Efficient B condition, peak VCD (9.38×10⁶ cells/mL) was reached at day 6 with a sharp drop in cell viability beginning day 7. Glucose in the E3D EffB and the E3D EffB2×Gln conditions was similar and fluctuated for both with an average low of 1.7 g/L and a high of 6.0 g/L. For the E3D EffB, glutamine was the lowest of all conditions and gradually decreased like the control (4.0 to 0.2 mmol/L). For E3D2×Gln, glutamine stayed very high and also fluctuated as the culture progressed with low of 1.0 mmol/L to a high of 5.3 mmol/L. The accumulation of lactate (0.3 to 2.0 g/L) and ammonium byproducts was observed; however, ammonium for E3D EffB reached a high of 5.0 mmol/L, whereas E3DEffB2×Gln had an excess buildup with a high of 18.0 mmol/L. This buildup appeared to be correlated to that sharp drop in cell viability for E3DEffB2×Gln. Osmolality remained relatively constant between 290 and 340 mOsmo, while pH decreased from 7.5 to 6.6 for both conditions.

Based on the data, it can be concluded that feeding Efficient B with double the amount of Glutamine every third day did not have a significant effect in terms of IVCD and peak VCD (Table 8) compared to feeding with the Efficient Feed B normally every third day. Even though the fastest doubling time is in the double Glutamine E3D condition, the normal E3D condition is similar.

TABLE 8
Double Glutamine Efficient B Study Peak VCD, IVCD, and Doubling
Times in CHO Feed 1 (n = 3), E3D EffB (n = 3) and E3D EffB2xGln
(n = 3). Both Efficient Feed B conditions outperformed the control
conditions. E3D (without additional glutamine) conditions displayed
similar doubling times to the 2x glutamine Efficient B
condition while increasing peak VCD and IVCD.

| Condition | CHO_Feed1 | E3D_EffB | E3D_EffB2xGln |
|---|---|---|---|
| Peak VCD (×10⁶ | 5.46 | 10.55 | 9.38 |
| cells/mL) | | | |
| IVCD | 43.92 | 50.63 | 47.63 |
| (×10⁶ cells × | | | |
| hr/mL) | | | |
| Doubling Time | 33.0 | 24.5 | 23.4 |
| (hours) | | | |

### Example 9—BalanCD® CHO Feeds

BalanCD® CHO Feed 2, 3, and 4 (“Feed 2”, “Feed 3”, and “Feed 4”, respectively) were studied to see if the growth profiles will compare or improve on the standard feeding solution of BalanCD® CHO Feed 1. They are all chemically-defined and animal origin-free, and the solution is made by Irvine Scientific.

### Methodology/Experimental Design

For this experiment, there were four conditions with different feeding solutions at 1× starting concentration—all with a base medium of CD-C4. For the first condition, which is the control, a standard feeding strategy was used with BalanCD® CHO Feed 1 (“CHO Feed1”). For the second through fourth conditions, BalanCD® CHO Feed 2, 3, and 4 were fed at the same interval and percentage as the control, respectively (“CHO Feed2”, “CHO Feed 3”, and “CHO Feed 4”, respectively). For all conditions, GlcNAc was supplemented to each of the flasks on Days 0 and 6 of approximately 1% of the working volume. (Table 9)

TABLE 9
Experimental conditions for early screening of BalanCD ® Feed
2, 3 & 4 using feeding schedules.

| Condition | CHO_Feed1 | CHO_Feed 2 | CHO_Feed 3 | CHO_Feed 4 |
|---|---|---|---|---|
| Feed Type | Feed 1 | Feed 2 | Feed 3 | Feed 4 |
| Volume (wv) % | 1) 5% | 1) 5% | 1) 5% | 1) 5% |
| | 2) 7% | 2) 7% | 2) 7% | 2) 7% |
| Schedule (days) | 1) 3-7 | 1) 3-7 | 1) 3-7 | 1) 3-7 |
| | 2) 8-13 | 2) 8-13 | 2) 8-13 | 2) 8-13 |

### Results & Discussion

In the first shake flask control condition (CHO Feed 1), peak VCD (6.77×10⁶ cells/mL) was reached at day 10 along with cell viability beginning to drop below 90% on day 8, and a 45% cell viability was observed at the end of the 14-day process. Glucose concentrations accumulated in the culture and had a steady increase after day 7, from 5.2 g/L to 10.6 g/L. Glutamine diminished by day 12 as the culture progressed 3.8 mmol/L to 1.9 mmol/L. Byproduct accumulation was observed for lactate increasing from 0.3 g/L to 1.2 g/L. Another byproduct accumulation was observed for ammonium starting from 0.9 mmol/L to 2.2 mmol/L, with a peak of 4.7 mmol/L. Osmolality (301-347 mOsmo) remained relatively constant while pH decreased as the culture continued, with an initial pH of 7.4 to 6.8 at harvest.

In the next shake flask condition using BalanCD® CHO Feed 2 (CHO Feed 2), a peak VCD (7.48×10⁶ cells/mL) was reached at day 9, followed by cell viability beginning to decline below 90% by day 8. Unlike the control condition (CHO Feed 1), glucose gradually increased in a tighter range (5.0 to 6.5 g/L) and may be the cause of a higher peak VCD. While CHO Feed 1 had depleted glutamine in the last stages of the process, glutamine for Feed 2 had a steady range from day 12-14 (3.0 to 3.1 mmol/L). The accumulation of lactate (0.3 to 1.7 g/L) and ammonium (0.9 to 2.1 with a peak at 5.1 mmol/L) byproducts were observed throughout the 12-day culture period. Osmolality remained relatively constant between 301 and 323 mOsmo, while pH decreased from 7.5 to 6.8.

In the third condition using BalanCD® CHO Feed 3 (CHO Feed 3), a peak VCD (7.57×10⁶ cells/mL) was reached at day 8, followed by cell viability beginning to decline below 90% by day 8. Glucose followed a similar trend as BalanCD® CHO Feed 1, but with a narrower range of 4.4 g/L to a high of 8.7 g/L. Glutamine had a large change in trend where levels began to deviate from other conditions starting on day 4 and continually increased with a peak at 5.8 mmol/L by day 12. The accumulation of lactate (0.2 to 2.4 g/L) and ammonium (0.9 to 6.0 mmol/L) was observed to be higher than the first two conditions, which is consistent with the trends observed for carbon sources in Feed 3. Osmolality was slightly higher than the first two conditions and began to fluctuate between 303 and 434 mOsmo—from the day of first Feed (day 3) and throughout the rest of the culture process—while pH decreased from 7.5 to 6.9.

Lastly, in the fourth condition using BalanCD® CHO Feed 4 (CHO Feed 4), a peak VCD (5.14×10⁶ cells/mL) was reached at day 6, followed by cell viability declining below 90% on day 6. Glucose is observed to be the highest of the tested conditions with a low of 3.2 g/L and a high of 16.0 g/L by day 14. The NOVA BIOPROFILE® 400 cell analysis system was unable to detect glutamine levels due to its high concentration mid-way through the culture period, but glutamine was observed to be the highest of all conditions with a high of 6.4 mmol/L on day 6. The accumulation of lactate (0.3 to 1.7 g/L) was observed to be somewhat similar the other conditions. The accumulation of ammonium (0.9 to 8.5 mmol/L) was only observed until day 9 because concentrations were too high for the NOVA instrument to detect in the succeeding days. Unlike all the other conditions, osmolality was not constant and had a high peak at 740 mOsmo on day 14, while pH decreased from 7.5 to 6.5.

When comparing these conditions, CHO Feed 3 was observed to have a higher peak VCD; however, CHO Feed 2 had greater IVCD and a faster doubling time VCD (Table 10). It was observed that the glutamine was not being metabolized by cells fed BalanCD® CHO Feed 3 and 4, and this is suspected to be the cause of the early death phase for these conditions. Since the glucose values for most conditions follow similar trends, the results are inconclusive; however, the slightly lower levels of glucose throughout the culture period for Feed 2 may be the cause of the increase in VCD when compared to the control. BalanCD® CHO Feed 2 using the standard strategy may be beneficial strategy to use for a bioreactor run because of its potential to reach a higher IVCD and quicker doubling time, compared to BalanCD® CHO Feed 3.

TABLE 10
BalanCD ® CHO Feeds Peak VCD, IVCD, and Doubling Times
in CHO Feed 1 (n = 3), CHO Feed 2 (n = 3), CHO Feed
3 (n = 3) and CHO Feed 4 (n = 3). BalanCD ® Feed
2 outperformed the other BalanCD ® CHO Feeds, including baseline Feed 1 conditions.

| Condition | CHO_Feed1 | CHO_Feed 2 | CHO_Feed 3 | CHO_Feed 4 |
|---|---|---|---|---|
| Peak VCD (×10⁶ | 6.77 | 7.48 | 7.57 | 5.14 |
| cells/mL) | | | | |
| IVCD | 56.5 | 63.04 | 51.9 | 5.19 |
| (×10⁶ | | | | |
| cells × hr/mL) | | | | |
| Doubling Time | 35.0 | 28.7 | 31.7 | 33.3 |
| (hours) | | | | |

### Example 11—BalanCD® CHO Feed 4 Studies

BalanCD® CHO Feed 4 was further investigated. Example 10 showed that there is a possibility that the cells were being overfed using BalanCD® CHO Feed 4. With this in mind, the next experiment was conducted using a less concentrated version of BalanCD® CHO Feed 4.

### Methodology/Experimental Design

For this experiment, there were three conditions with different feeding strategies. All were in the medium CD-C4. The first condition was the control using a standard feeding strategy of BalanCD® CHO Feed 1 (“CHO F1”). The second condition fed BalanCD® CHO Feed 4 every other day at 4% working volume (“SF E2D F4”). This is the recommended strategy of Feed 4 suggested by Irvine Scientific. The third condition fed BalanCD® CHO Feed 4 every third day also at 4% working volume (“SF E3D F4”). The concentration of Feed 4 for both SF E2D F4 and SF E3D F4 was 0.8× as opposed to the previously used 1.0× because of the possibility of overfeeding. (Table 11)

TABLE 11
Experimental conditions evaluating different feeding
strategies utilizing BalanCD ® Feed 4.

| Condition | CHO F1 | SF_E2D_F4 | SF_E3D_F4 |
|---|---|---|---|
| Feed Type | Feed 1 | Feed 4 0.8X | Feed 4 0.8X |
| Volume (wv) % | 1) 5% | 4% | 4% |
| | 2) 7% | | |
| Schedule (days) | 1) 3-7 | 2, 4, 6, | 3, 6, 9, 12 |
| | 2) 8-13 | 8, 10, 12 | |

### Results & Discussion

This experiment was cut short because the experimental flasks reached a percent viability of below 10%. Data reported in this Example is reported through 9 days of culture.

In shake flask control condition (CHO F1), peak VCD (6.25×10⁶ cells/mL) was reached at day 7 along with a drop in viability. Glucose started to accumulate after day 4, which is also when VCD started to stabilize. In comparison, there is no correlation between the VCD and glutamine since glutamine levels fluctuated between the levels of 1.26 mmol/L and 1.87 mmol/L. The culture environment shows no abnormality since osmolality stayed within the range of 300-325 mOsmo and pH settled to 6.74.

There were no significant differences between two experimental conditions (SF_E2D_F4 and SF_E3D_F4); therefore, they are discussed together. Feed 4 reached peak VCD (9.90 and 11.32×10⁶ cells/mL for SF_E2D_F4 and SF_E3D_F4, respectively) at day 7. The percent viability dropped the next day (90.5 to 56.1% and 92.1 to 47.4% for SF_E2D_F4 and SF_E3D_F4, respectively). The drop in percent viability can be attributed to the glucose levels. Glucose levels reached below 1 g/L for both Feed 4 conditions by day 7. Usually, a healthy environment has at least 3 g/L of glucose in the culture. A waste product of glucose, lactate, started to decrease once glucose was depleted. There was a slight uptick in lactate for E2D_F4 due to feeding on day 8. A similar drop was not seen in glutamine. In fact, glutamine was observed to accumulate after day 5. Glutamine consumption briefly increased after day 8, which was attributed to the depletion of lactate. A waste product of glutamine, ammonium, was observed to rise after day 6. The osmolality trended up reaching about 370 which was slightly above the control. Increases in pH were observed lactate decreased, and the final value of pH was about 7.48. The elevated ammonium and pH levels are not likely to contribute to the decrease in VCD, but rather were likely a result of it.

When comparing the conditions through 9 days of culture, Feed 4 had the fastest doubling time, greater IVCD, and peak VCD, as shown in Table 12. Feed 4 did a better job of metabolizing glucose that results in a higher peak VCD.

TABLE 12
Feed 4 Peak VCD, IVCD, and Doubling Times in
SF F1 (n = 3), SF E2D F4 (n = 3),
and SF E3D F4 (n = 3) conditions. BalanCD ® Feed
4 conditions notably outperformed control conditions
for IVCD and peak VCD.

| Condition | SF_F1 | SF_E2D_F4 | SF_E3D_F4 |
|---|---|---|---|
| Peak VCD (×10⁶ | 6.25 | 9.90 | 11.32 |
| cells/mL) | | | |
| IVCD | 32.70 | 39.24 | 38.88 |
| (×10⁶ cells × hr/mL) | | | |
| Doubling Time | 32.70 | 32.86 | 32.64 |
| (hours) | | | |

### Example 12—Further BalanCD® CHO Feed 4 Studies

Based on the results of the Example 11, another study was conducted to investigate extending the stationary phase. In total, there were 5 conditions. The first condition was the control using BalanCD® CHO Feed 1 with a standard strategy (“CHO F1”). The second condition was 0.8× concentration of BalanCD® CHO Feed 4 with glucose and glutamine supplement after day 7 (“Supp F4”). The third condition was 0.8× concentration of Feed 4 every other day, transitioning to every day (“E2D_F4”). The fourth condition was 0.8× concentration of Feed 4 that was fed every third day with increasing percent working volume (“E3D_F4”). Finally, the last condition was 1× concentration of BalanCD® CHO Feed 4 fed every day (“1× F4”). (Table 13)

TABLE 13
Experimental conditions for different feeding strategies
utilizing BalanCD ® Feed 4.

| Condition | CHO_F1 | Supp_F4 | E2D_F4 | E3D_F4 | 1X_F4 |
|---|---|---|---|---|---|
| Feed Type | Feed 1 | 1) Feed 4 0.8X | Feed 4 0.8X | Feed 4 0.8X | Feed 4 1X |
| | | 2) Feed 4 1X W/ | | | |
| | | gluc & gln | | | |
| Volume | 1) 5% | 4% | 4% | 1) 4% | 4% |
| (wv) % | 2) 7% | | | 2) 6% | |
| | | | | 3) 8% | |
| Schedule | 1) 3-7 | 1) 3-7 | 3, 5, 6, | 1) 3 | 3-13 |
| (days) | 2) 8-13 | 2) 8-13 | 7-14 | 2) 5 | |
| | | | | 3) 7, 9, | |
| | | | | 11, 13 | |

### Results and Discussion

For the control condition (CHO F1), peak VCD was reached at day 8, followed by cell viability beginning to decline below 90% on the same day. Glucose declined steadily until day 3 where it started to stabilize around 5 g/L. Glutamine was depleted by day 4. Waste products such as lactate and ammonium stayed within healthy ranges. Osmolality and pH also stayed within expected ranges.

In the supplemented shake flasks (Supp F4), a peak VCD of 11.26×10⁶ cell/mL was observed on day 8. The following day percent viability dropped below 90% and continued to drop slowly. Glucose levels were maintained around 2.30 to 3.29 g/L, dropping briefly to 1.22 g/L. Glutamine does not seem to be metabolized, as accumulation was observed after day 7. Glutamine levels reach as high as 9.80 mmol/L. Lactate stabilized around 3 g/L, which is a normal level. Ammonium, increased after day 8. Ultimately, ammonium reached levels as high as 21.18 g/L (8 g/L is considered to be detrimental to VCD). Osmolality peaked at 578 mOsm/L, whereas healthy levels are between 280-340 mOsm/L. The pH stabilized around 6.8, which is a healthy number.

The last three conditions, E2D_F4, E3D_F4, and 1× F4, did not see much variation and are discussed together as “unsupplemented conditions”. The unsupplemented conditions reached a peak VCD of 12.54×10⁶ cell/mL on day 8. A large drop off in viability was observed the next day—percent viability went from above 90% to 50% in one day. The level of glucose dropped below 1 g/L by day 7. This is similar to Example 11, as glucose has a direct impact on VCD. Similarities between the unsupplemented conditions and Example 11 were also observed, as there is no correlation between glutamine and feeding strategy, lactate concentration stabilized then fell, and ammonium accumulated. Osmolality and pH both trends upwards. This again is similar to the previous Example.

As shown in Table 14, 1× F4 had the highest peak VCD and fastest doubling time. Of the experimental Feed 4 flasks, however, supplemented Feed 4 (Supp F4) performed the best in IVCD. It did not die as quickly as the unsupplemented conditions. Additional Examples are focused around supplementing Feed 4 by adding glucose and not glutamine. Glutamine creates an excess of ammonium and is generally not metabolized by the cells.

TABLE 14
Further Feed 4 study Peak VCD, IVCD, and Doubling Times
in CHO F1 (n = 3), Supp F4 (n = 3), E2D_F4 (n =
3), E3D_F4 (n = 3) and 1X_F4 (n = 3).
Despite a higher IVCD achieved in control conditions,
Feed 4 conditions demonstrated strong peak VCD throughout.

| Condition | CHO_F1 | Supp_F4 | E2D_F4 | E3D_F4 | 1X_F4 |
|---|---|---|---|---|---|
| Peak VCD | 8.07 | 11.26 | 11.56 | 12.54 | 13.72 |
| (×10⁶ | | | | | |
| cells/mL) | | | | | |
| IVCD | 69.55 | 65.54 | 46.78 | 44.04 | 60.54 |
| (×10⁶ cells × | | | | | |
| hr/mL) | | | | | |
| Doubling | 29.97 | 30.81 | 31.93 | 33.33 | 29.39 |
| Time (hours) | | | | | |

### Example 13—Glucose Corrections in Shake Flasks

A study was conducted to investigate the maintenance of glucose concentration using a bolus feed of BalanCD® CHO Feed 4 every other day.

### Methodology/Experimental Design

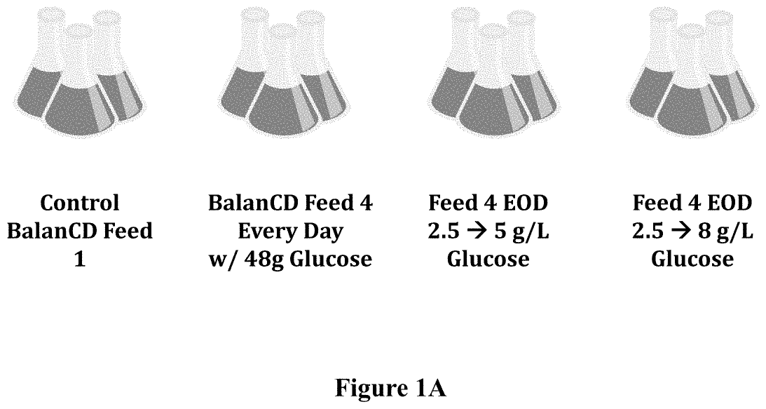

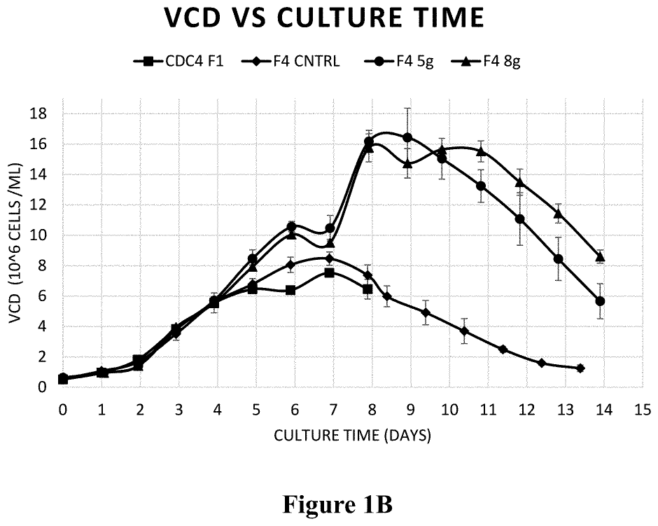

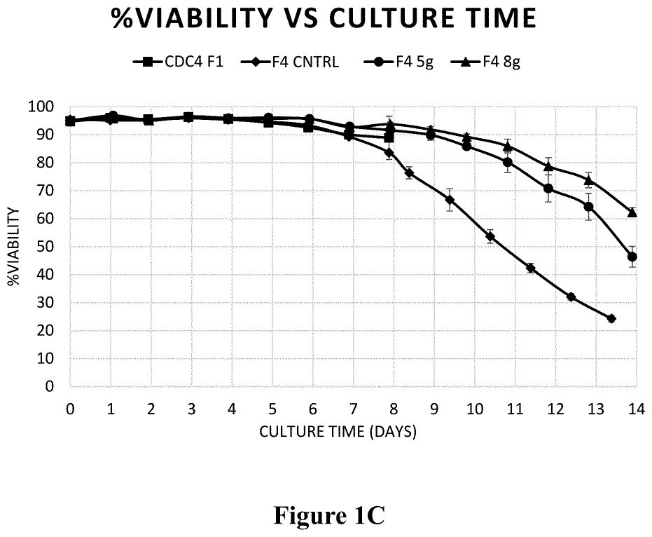

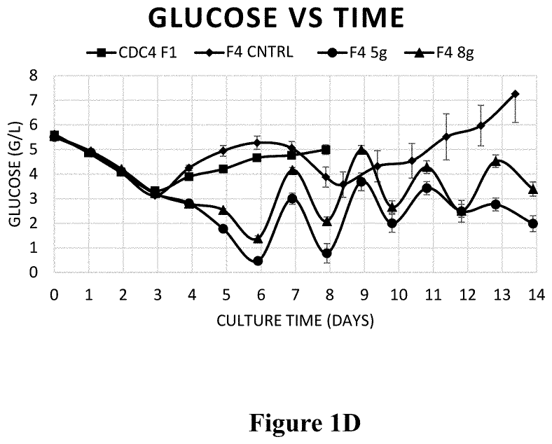

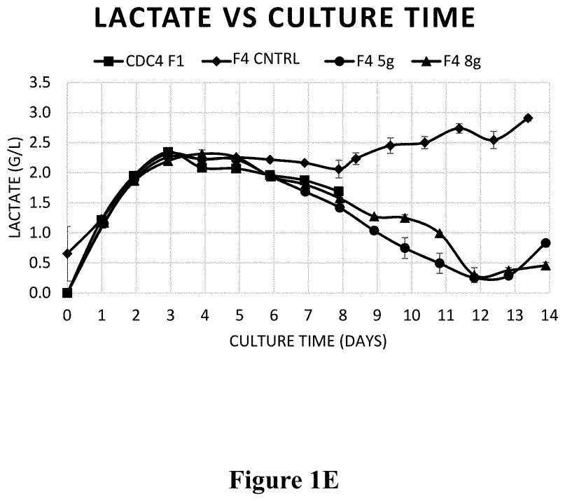

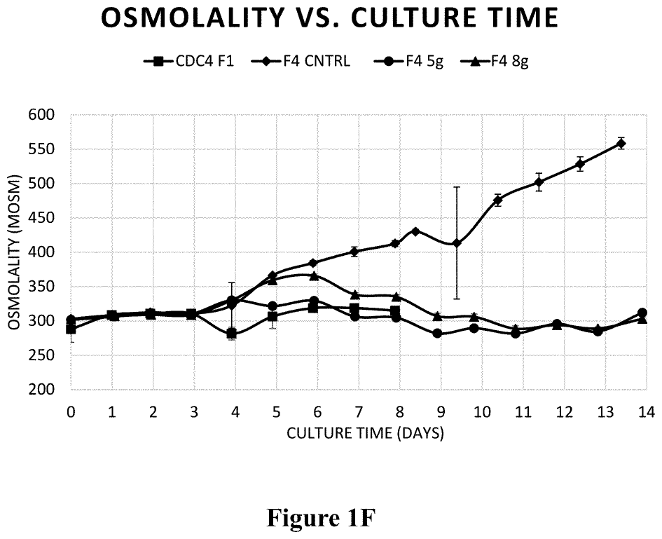

In total, there were 4 conditions. The first condition was the control using BalanCD® CHO Feed 1 with a standard strategy (“CDC4 F1”). The second condition was BalanCD® CHO Feed 4 fed every day with 48 g/L glucose (“F4 CNTRL”). The third condition was 0.8× concentration of Feed 4 every other day, with a correction to 5 g/L glucose (“F4 5 g”). The fourth condition was 0.8× concentration of Feed 4 that was fed every other day, with a correction to 8 g/L glucose (“F4 8 g”). (FIG. 1A, Table 15)

TABLE 15

| CDC4 F1 | Current Strategy |
|---|---|
| F4 CNTRL | Feed 4 w/48 g/l gluc ED |
| F4 5 g | Feed 4 EOD at 0.8X |
| | Correct to 5 g/l gluc |
| F4 8 g | Feed 4 EOD at 0.8X |
| | Correct to 8 g/l gluc |

### Results & Discussion

The results of this experiment are shown in FIG. 1B-1F. In each graph, CDC4 F1 data is shown as squares; F4 CNTRL data is shown as diamonds; F4 5 g data is shown as circles; F4 8 g data is shown as triangles. FIG. 1B is a plot of viable cell density (VCD) vs. culture time. FIG. 1C is a plot of percent viability vs. culture time. FIG. 1D is a plot of glucose concentration (g/L) vs. culture time. FIG. 1E is a plot of lactate concentration (g/L) vs. culture time. FIG. 1F is a plot of osmolality (mOsm) vs. culture time.

Table 16 shows the doubling time, IVCD, and Peak VCD for each of the four conditions. Glucose corrections maintained similar culture characteristics while showing positive improvements to growth characteristics. The glucose corrections are helpful for BalanCD® CHO Feed 4 strategy. Different glucose corrections were negligible.

TABLE 16

| Condition | Doubling time | IVCD | Peak VCD |
|---|---|---|---|
| CDC4 F1 | 24.24 | 35.64 | 7.53 |
| F4 CNTRL | 26.65 | 58.35 | 8.47 |
| F4 5 g/L | 28.11 | 123.78 | 16.43 |
| F4 8 g/L | 26.56 | 129.67 | 15.76 |

### Feed Performance Assessment

After the feed investigation phase, selected feeding strategies were scaled up to a MOBIUS® SUB STR (3 L) bioreactor for further evaluation. Along with validating improved process characteristics, product characteristics were evaluated for any potential increase in titer productivity while maintaining consistent product quality. The following Examples will provide a table of parameters, and relevant results from each experiment, followed by a brief discussion of result and further experiments if applicable.

### Example 14—Feed 2 Bioreactor Study

Based on previous Examples, Feed 2 conditions sometimes demonstrated stronger growth profiles. Thus, a scale up assessment was performed to validate process characteristics and potential improvements in product characteristics.

### Methodology/Experimental Design

For this experiment, two bioreactors (BRX) were run with a base medium of CD-C4. For the first condition, which is the control (BR F1), a standard feeding strategy was used with BalanCD® CHO Feed 1. For the second condition, the bioreactor with Feed 2 (BR F2) was used. (Table 17) Control shake flasks were also run with BalanCD® CHO Feed 1, and BalanCD® CHO Feed 2.

TABLE 17
Experimental conditions for MOBIUS ® BRX
run evaluating BalanCD ® Feed 2
using a standard feeding strategy.

| Condition | BR F1 | BR F2 |
|---|---|---|
| Feed Type | Feed 1 | Feed 2 |
| Volume (wv) % | 1) 5% | 1) 5% |
| | 2) 7% | 2) 7% |
| Schedule (days) | 1) 3-7 | 1) 3-7 |
| | 2) 8-13 | 2) 8-13 |

### Results & Discussion

In the control condition (BR F1), peak VCD (10.21×10⁶ cells/mL) was reached at day 9, and cell viability began to decrease. Glucose concentrations increased in the culture, whereas glutamine diminished as the culture progressed—from 5.99 g/L to 7.18 g/L and 4.39 mmol/L to 3.11 mmol/L, respectively. Byproduct accumulation was observed for lactate and ammonium, increasing from 0.46 g/L to 1.41 g/L and 1.07 mmol/L to 2.17 mmol/L, respectively. Osmolality fluctuated (233-397 mOsm) throughout the culture period but did not seem to have a significant effect. The pH did not fluctuate and stayed within a narrow range (6.8-7). The second condition followed similar process characteristics.

In the experimental condition for Feed 2 (BR F2), a peak VCD (9.43×10⁶ cells/mL) was reached at day 9, cell viability declined below 90% by day 11. Like the control condition (BR F1), glucose slowly increased (5.84 to 6.2 g/L) and glutamine gradually decreased (4.51 to 2.88 mmol/L). The accumulation of lactate (0.23 to 1.97 g/L) and ammonium (0.82 to 1.87 mmol/L) byproducts were observed throughout the 13-day culture period. Osmolality fluctuated throughout the culture period, but the fluctuations could not explain the drop in VCD. The pH was maintained around (6.8-7) and thus had no effect on the cell culture.

When comparing both conditions, BR F1 observed a faster doubling time, greater IVCD, and peak VCD (Table 18). BR F2 was slightly lower in each of those aspects, but it is worthy to note that BR F2 has a longer stationary phase as opposed to BR F1. Most values were fairly similar in terms of metabolites, but the lactate values for BR F2 were slightly higher.

TABLE 18
Bioreactor Feed 2 study in VCD, IVCD, and Doubling Times
in BR Feed 1 and BR Feed 2. Despite results from shake
flask conditions, the current control process outperformed
BalanCD ® Feed 2 experimental conditions.

| Condition | BR F1 | BR F2 |
|---|---|---|
| Peak VCD | 10.21 | 9.43 |
| (×10⁶ cells/mL) | | |
| IVCD | 65.04 | 64.35 |
| (×10⁶ cells × hr/mL) | | |
| Doubling Time (hours) | 32.12 | 38.58 |

### Example 15—Feed 4 Bioreactor Study

A bioreactor study comparing BalanCD® CHO Feed 1 versus Feed 4 was conducted as a result of the Feed 4 shake flask studies. The supplement is made by Irvine Scientific, chemically defined, animal component-free, and contains no protein hydrolysates or any other undefined components.

### Methodology/Experimental Design

Two three-liter, single-use MOBIUS® bioreactors were inoculated at a viable cell density (VCD) of 0.5 million cells per milliliter (mL) with an after-inoculation working volume of 1100 mL. Each bioreactor had an identical cell source, and three respective control shake flasks at an after-inoculation working volume of 50 mL. One bioreactor and its control shake flasks were fed with 5% of their working volume of Feed 1 from day 3 to day 8, and 7% from day 9 to day 14 (“BR F1” and “SF F1” for the bioreactor and shake flasks respectively). This is considered the control for this experiment, utilizing a standard feeding strategy. The other bioreactor and its control shake flasks were fed with 4% of their working volume of 0.8× Feed 4 from day 3 to day 6, and 4% of 1.0× Feed 4 with 2.0× glutamine and glucose concentrations from day 7 to day 14 (“BR F4” and “SF F4” for the bioreactor and shake flasks, respectively) (Table 19).

TABLE 19
Experimental conditions comparing the control fed-batch process to
an experimental Feed 4 process in MOBIUS ® BRX runs.

| Condition | BR F1 & SF F1 | BR F4 & SF F4 |
|---|---|---|
| Feed Type | Feed 1 | Feed 4 |
| Volume (wv) % | 1) 5%, 1.0x | 1) 4%, 0.8x |
| | 2) 7%, 1.0x | 2) 4%, 1.0x, |
| | | 2x gln & gluc |
| Schedule (days) | 1) 3-8 | 1) 3-6 |
| | 2) 9-13 | 2) 7-13 |

### Results & Discussion

In the control conditions (BR F1, SF F1), peak viable cell density was accomplished at about day 9 in the bioreactor the respective shake flasks (11.26 and 8.07×10⁶ cells/mL, respectively). Cell viability subsequently began to drop below 90% until harvest, where there was a 50.9% cell viability in the bioreactor and 50.3% in the shake flasks. Glucose concentrations stabilized around day 4 and began slightly accumulating after day 9, whereas glutamine was depleted at about day 6. Additionally, Feed 1 supported lower inhibitory metabolite production (than Feed 4) as observed after day 8 for the lactate and ammonium levels. As a result of these main metabolite trends, the osmolality remained relatively constant between 290-370 mOsmo, with day 2 and 4 of the bioreactor having a fluctuation of about 430 mOsm and then returning to the original range directly after. The pH was maintained in the bioreactor between 6.8 and 7.2 and started at 7.6 in the shake flasks and decreasing until 6.95 at harvest.

In the experimental condition (BR F4, SF F4), peak viable cell density was reached at day 8 in the bioreactor and day 7 in the shake flasks (14.2 and 8.07×10⁶ cells/mL, respectively). The cell viability proceeded to drop below 90% directly after these peaks, with a 12.0% viability at harvest in the bioreactor and 13.4% in the shake flasks. Glucose steadily decreased to about 1 g/L with a slight increase thereafter, however glutamine levels had a large accumulation after day 6. Feed 4 had an accumulation of inhibitory metabolites after day 8; both lactate and ammonium were accumulated. Consequently, the osmolality experienced a stark increase from 340.33-576.67 mOsmo starting at day 6 to harvest time in the bioreactor. The pH was similar to that of Feed 1.

When comparing the bioreactor conditions, Feed 4 displayed a faster doubling time and peak VCD (Table 20). Feed 1 resulted in a higher IVCD. It was observed that the glutamine concentration of the Feed 4 condition tended to rise drastically after the peak VCD was reached when compared to the Feed 1 strategy. Additionally, lactate and ammonium levels also rose higher in Feed 4 than Feed 1. These accumulations resulted in a higher osmolality.

TABLE 20
BalanCD ® CHO Feed 4 Study Peak VCD, IVCD, and
Doubling Times in Feed 1 Bioreactor (n = 1), Feed
1 Shake Flasks (n = 3), Feed 4 Bioreactor (n =
1) and Feed 4 Shake Flasks (n = 3). Despite a higher
IVCD achieved in the control conditions, Feed 4 conditions
demonstrated a strong exponential phase.

| Condition | BR F1 | SF F1 | BR F4 | SF F4 |
|---|---|---|---|---|
| Peak VCD (×10⁶ | 11.25 | 8.07 | 14.20 | 11.26 |
| cells/mL) | | | | |
| IVCD | 84.84 | 70.76 | 77.56 | 65.45 |
| (×10⁶ cells × | | | | |
| hr/mL) | | | | |
| Doubling Time | 37.3 | 27.9 | 29.8 | 28.7 |
| (hours) | | | | |

### Example 16—Glucose Corrections in Bioreactors

A study was conducted to validate promising growth characteristics and potential titer improvements of glucose corrections with BalanCD® CHO Feed 4 bolus feeding.

### Methodology/Experimental Design

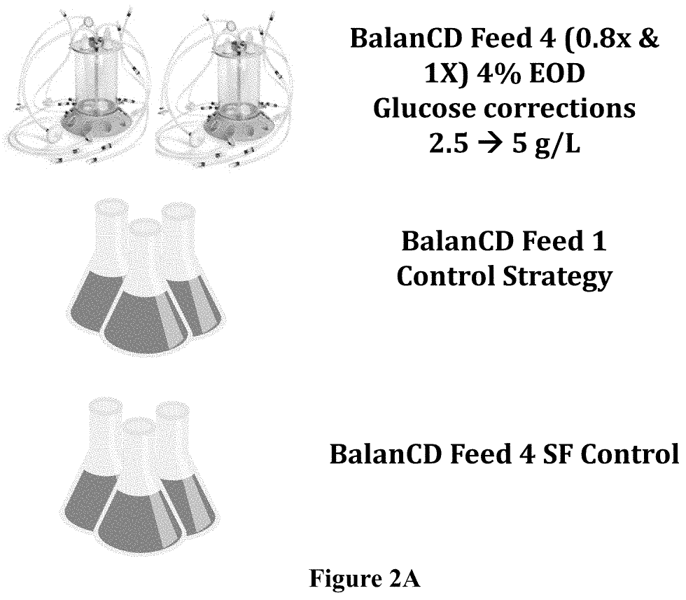

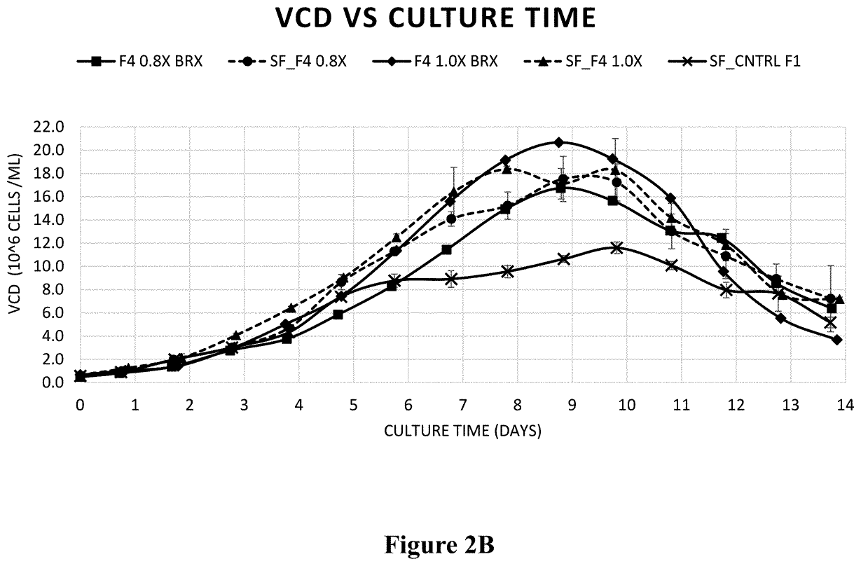

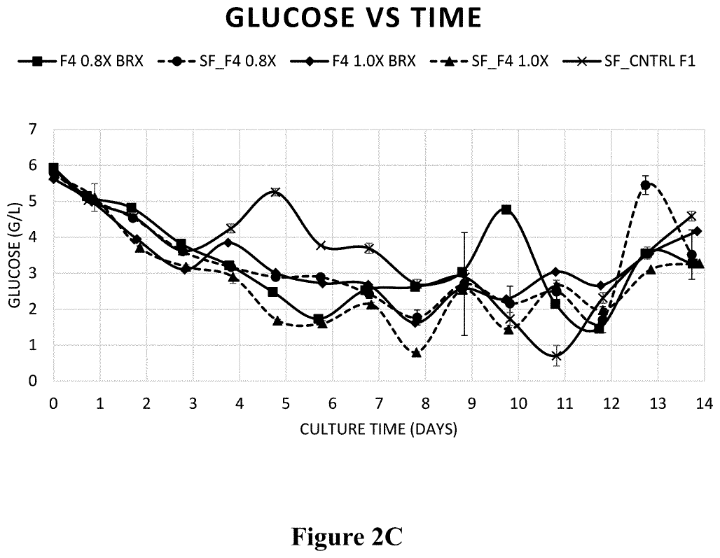

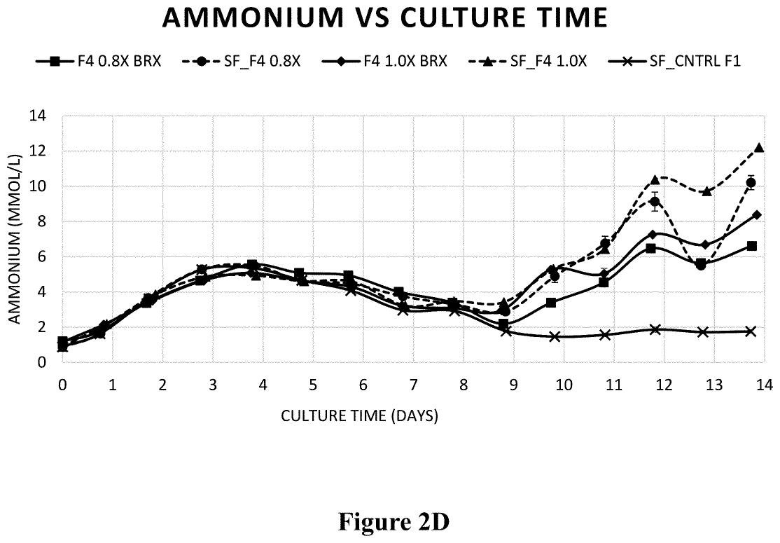

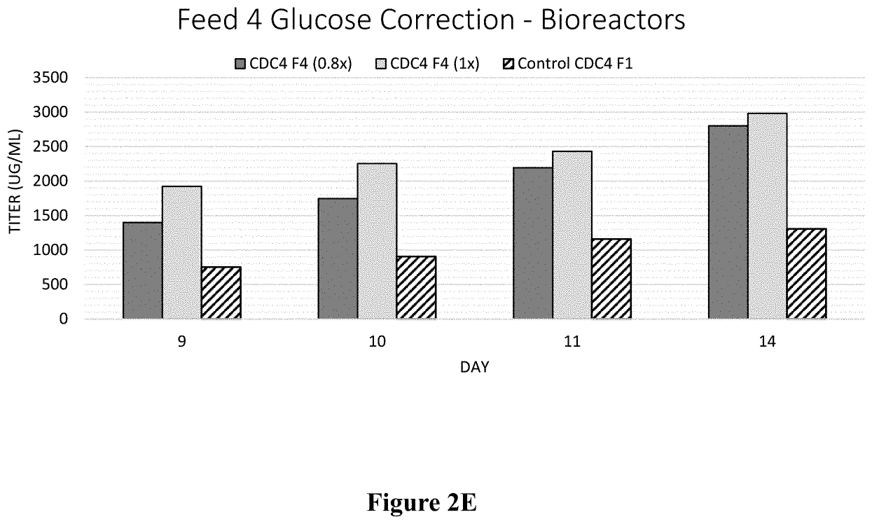

In total, there were 5 conditions. The first condition was the control using BalanCD® CHO Feed 1 with a standard strategy in a shake flask (“SF CNTRL F1”). The second and third condition was 0.8× concentration of Feed 4 every other day, with a correction to 5 g/L glucose in a bioreactor and in shake flasks (“F4 0.8X BRX” and “SF F4 0.8×”, respectively). The fourth and fifth conditions were 1.0X concentration of Feed 4 every other day, with a correction to 5 g/L glucose (“F4 1.0X BRX” and “SF F4 1.0×”, respectively). (FIG. 2A, Table 21)

TABLE 21

| | | F4 0.8X BRX and | BR F4 & |
|---|---|---|---|
| Condition | SF_CNTRL F1 | SF F4 0.8X | SF F4 |
| Feed Type | Feed 4 | Feed 4 | Feed 4 |
| Volume (wv) % | 1) 5%, 1.0x | 4% | 4% |
| | 2) 7%, 1.0x | | |
| Schedule (days) | 1) 3-8 | 3, 5, 7, | 3, 5, 7, |
| | 2) 9-13 | 9, 11, 13 | 9, 11, 13 |

### Results & Discussion

The results of this experiment are shown in FIG. 2B-2D. In each graph, SF CNTRL F1 data is shown as “X” markers with a solid line connection; F4 0.8X BRX data is shown as squares with a solid line connection; SF F4 0.8X data is shown as circles with a dashed line connection; F4 1.0X BRX data is shown as diamonds with a solid line connection; and SF F4 1.0X data is shown as triangles with a dashed line connection. FIG. 2B is a plot of viable cell density (VCD) vs. culture time. FIG. 2C is a plot of glucose concentration (g/L) vs. culture time. FIG. 2D is a plot of ammonium concentration (mmol/L) vs. culture time. FIG. 2E is a bar plot of product titer produced in a 0.8X Feed 4 bioreactor, a 1.0X Feed 4 bioreactor, and a control Feed 1 shake flask over time. In FIG. 2E the titer values (μg/mL) are as follows, Day 9: F4 0.8X, F4 1.0X, and CNTRL F1 are 1400, 1923, and 752, respectively; Day 10: F4 0.8X, F4 1.0X, and CNTRL F1 are 1747, 2255, and 907, respectively; Day 11: F4 0.8X, F4 1.0X, and CNTRL F1 are 2194, 2431, and 1160, respectively; and Day 14: F4 0.8X, F4 1.0X, and CNTRL F1 are 2801, 2981, and 1308, respectively.

Table 22 shows the doubling time, IVCD, and Peak VCD for the F4 0.8X BRX, F4 1.0X BRX, and SF CNTRL F1 conditions. The F4 1.0X conditions showed improved growth characteristics over the F4 0.8X conditions, with the notable exception of the control having an improved doubling time over both. A higher concentration of feed led to increased IVCD and peak VCD.

TABLE 22

| Condition | Doubling time | IVCD | Peak VCD |
|---|---|---|---|
| F4 (0.8X) Gluc | 27.25 | 102.75 | 16.74 |
| F4 (1.0X) Gluc | 32.20 | 137.29 | 20.67 |
| F1 CNTRL | 26.94 | 95.26 | 11.60 |

### Example 17—HyClone™ ActiPro™ Medium Bioreactor

A study was conducted to investigate the effects of medium adaptation and medium switch at experimental scale (production).

### Methodology/Experimental Design

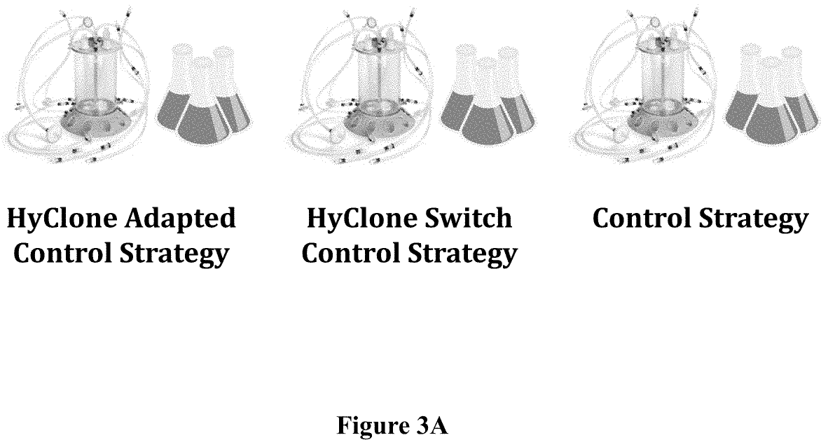

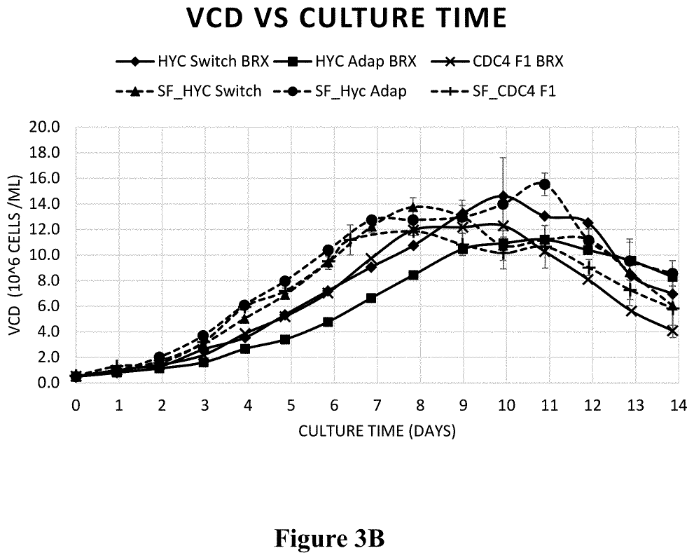

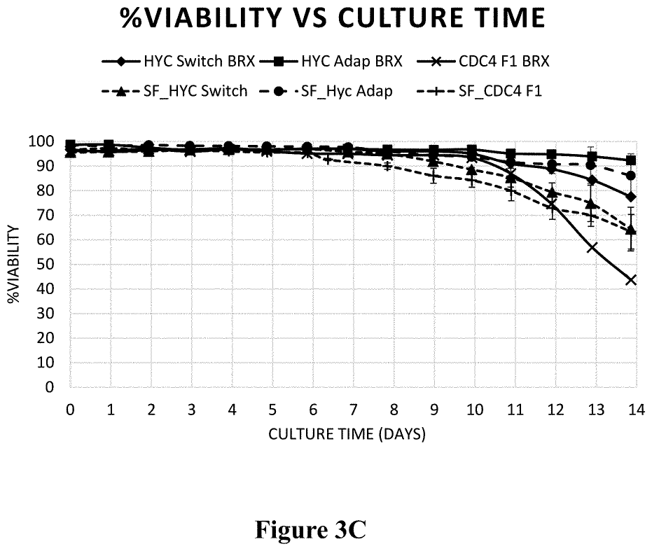

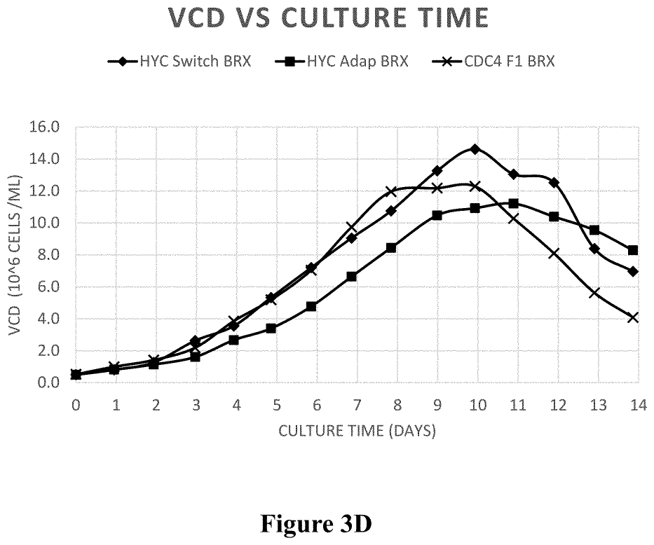

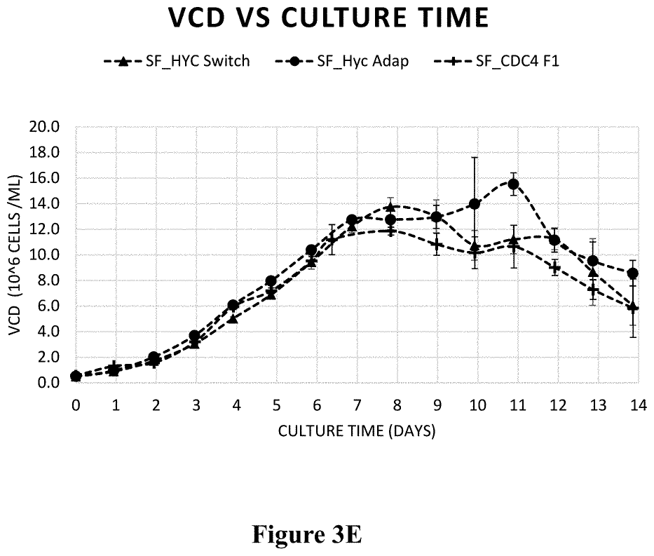

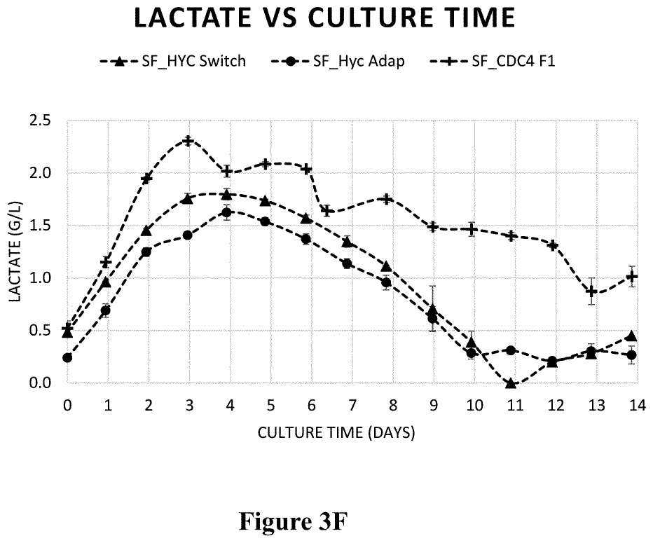

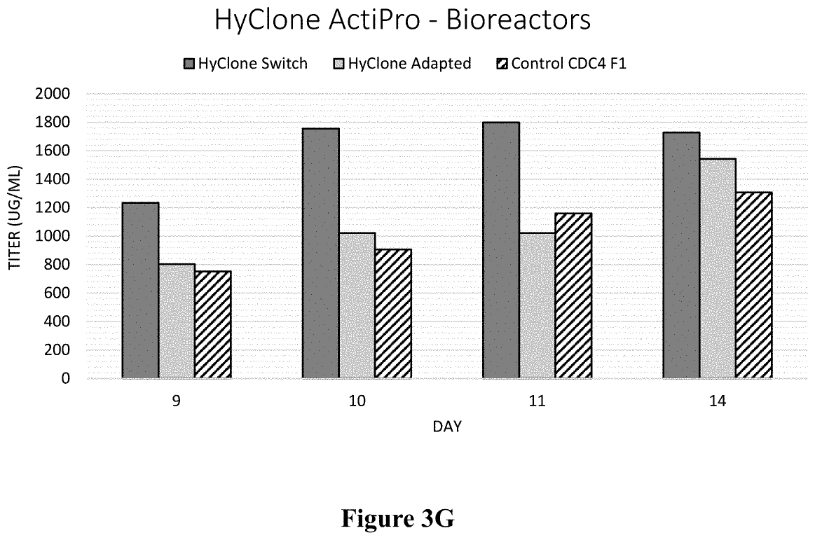

In total, there were 6 conditions. The first condition and the second condition was a standard strategy, in a bioreactor and in shake flasks (“CDC4 F1 BRX” and “SF CDC4 F1”, respectively). The third condition and the fourth condition was HyClone™ adapted control strategy, in a bioreactor and in shake flasks (“HYC Adap BRX” and “SF Hyc Adap”, respectively). The fifth condition and the sixth condition was HyClone™ switch strategy, in a bioreactor and in shake flasks (“HYC Switch BRX” and “SF HYC Switch”, respectively). (FIG. 3A, Table 23)

TABLE 23

| | CDC4 F1 | HYC Adap | HYC Switch |
|---|---|---|---|
| | BRX & | BRX & | BRX & |
| Condition | SF_CNTRL F1 | SF Hyc Adap | SF HYC Switch |
| Feed Type | Feed 1 | Feed 1 | Feed 1 |
| Volume | 1) 5%, 1.0x | 1) 5%, 1.0x | 1) 5%, 1.0x |
| (wv) % | 2) 7%, 1.0x | 2) 7%, 1.0x | 2) 7%, 1.0x |
| Schedule | 1) 3-8 | 1) 3-8 | 1) 3-8 |
| (days) | 2) 9-13 | 2) 9-13 | 2) 9-13 |

### Results & Discussion

The results of this experiment are shown in FIG. 3B-3F. In each graph, HYC Adap BRX data is shown as squares with a solid line connection; SF HYC Adap data is shown as circles with a dashed line connection; HYC Switch BRX data is shown as diamonds with a solid line connection; SF HYC Switch data is shown as triangles with a dashed line connection; CDC4 F1 BRX data is shown with “X” markers and a solid line connection; and SF CNTRL F1 data is shown with “+” markers and a dashed line connection. FIG. 3B is a plot of viable cell density (VCD) vs. culture time for all six conditions. FIG. 3C is a plot of cell viability vs. culture time for all six conditions. FIG. 3D is VCD vs. culture time for the bioreactor conditions. FIG. 3E is a plot of VCD vs. culture time for the shake flask conditions. FIG. 3F is a plot of lactate concentration (g/L) vs. culture time for the shake flask conditions. FIG. 3G is a bar plot of product titer produced in a HyClone™ Switch bioreactor, a HyClone™ Adapted bioreactor, and a control Feed 1 bioreactor over time.

In FIG. 3G the titer values (μg/mL) are as follows, Day 9: HYC Switch, HYC Adap, and CDC4 F1 are 1234, 803, and 752, respectively; Day 10: HYC Switch, HYC Adap, and CDC4 F1 are 1755, 1022, and 907, respectively; Day 11: HYC Switch, HYC Adap, and CDC4 F1 are 1799, 1022, and 1160, respectively; and Day 14: HYC Switch, HYC Adap, and CDC4 F1 are 1728, 1542, and 1308, respectively.

Table 24 shows the doubling time, IVCD, and Peak VCD for the HyClone™ Adapted, HyClone™ Switch, and CNTRL F1 conditions. A medium switch observed lower lactate metabolite levels. Experimental conditions also outperformed in terms of cell grown and maintenance of cell viability.

TABLE 24

| Condition | Doubling time | IVCD | Peak VCD |
|---|---|---|---|
| HyClone ™ Switch | 26.63 | 110.83 | 14.61 |
| HyClone ™ Adapted | 24.57 | 123.76 | 15.53 |
| CD-C4 F1 | 30.45 | 103.11 | 12.29 |

### Example 18—Efficient Feed B

A study was conducted to analyze and investigate the performance of cells with a separate feed in the same initial media.

### Methodology/Experimental Design

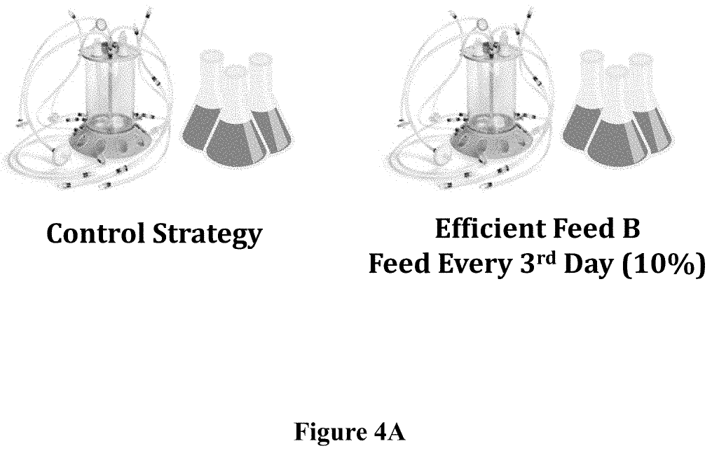

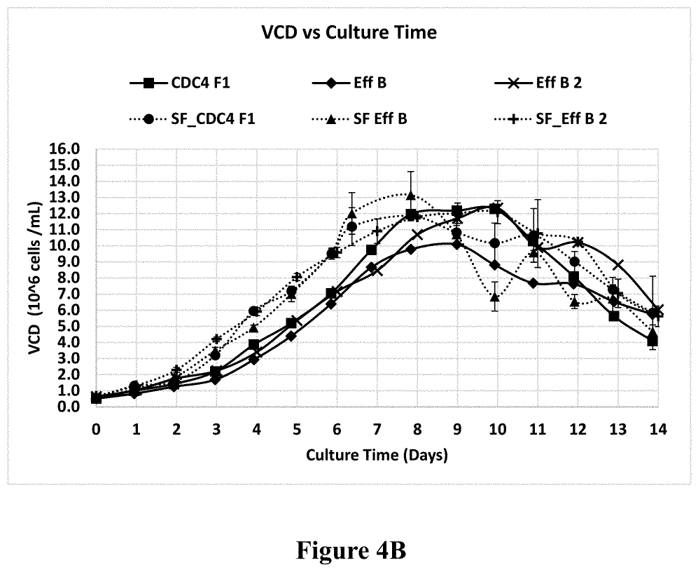

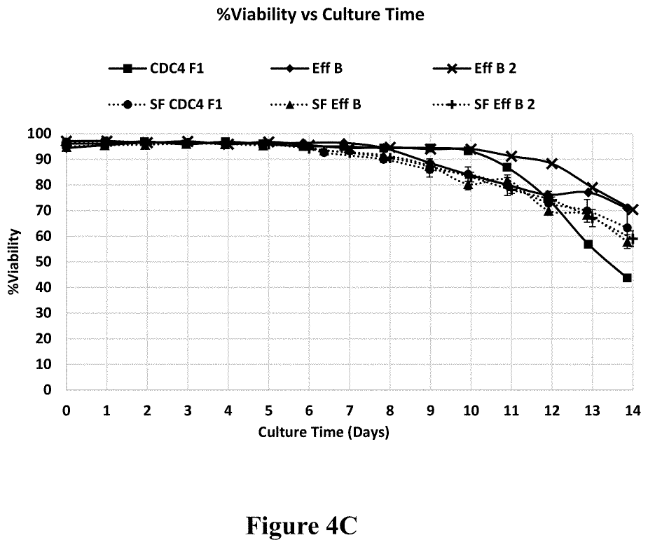

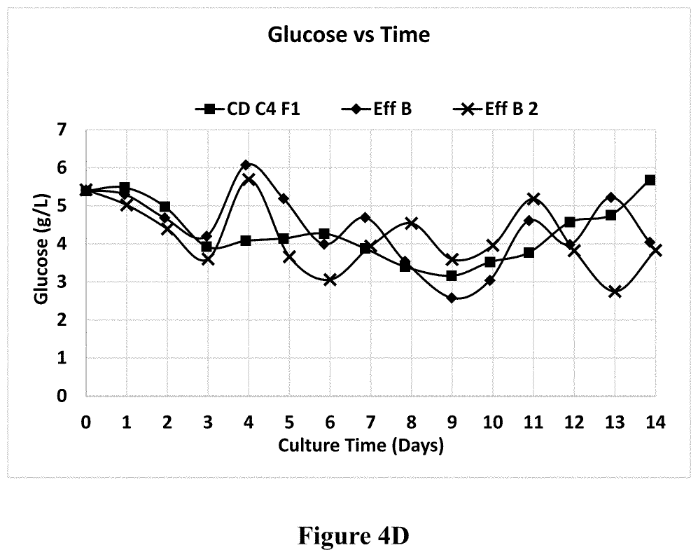

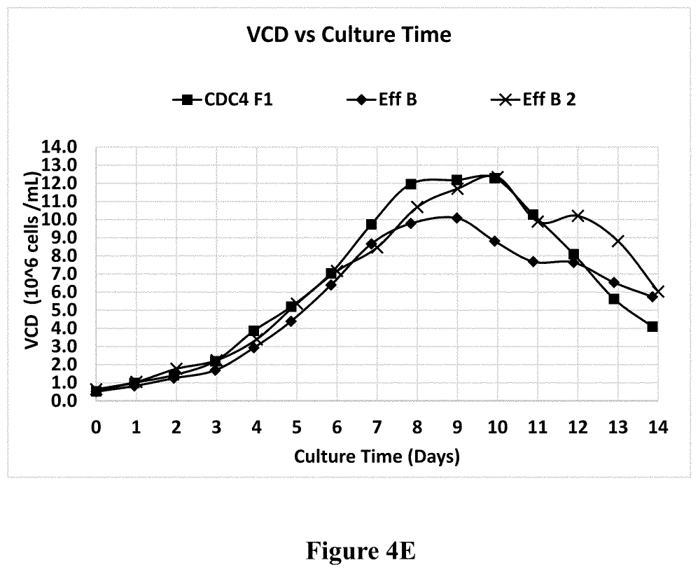

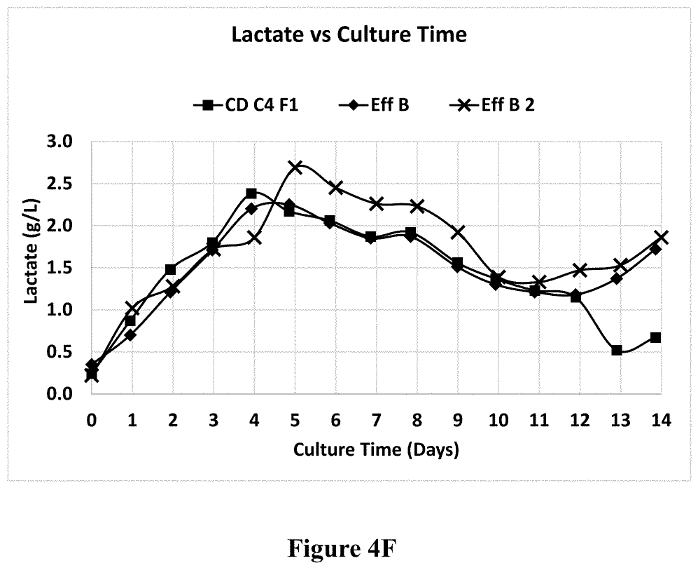

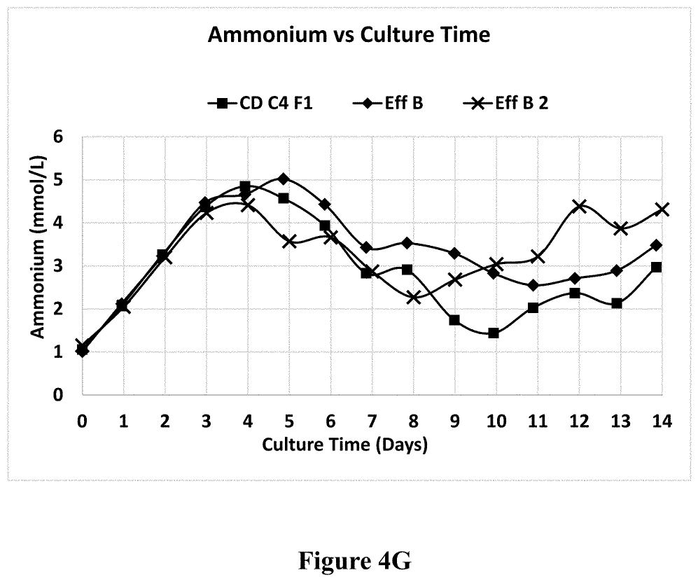

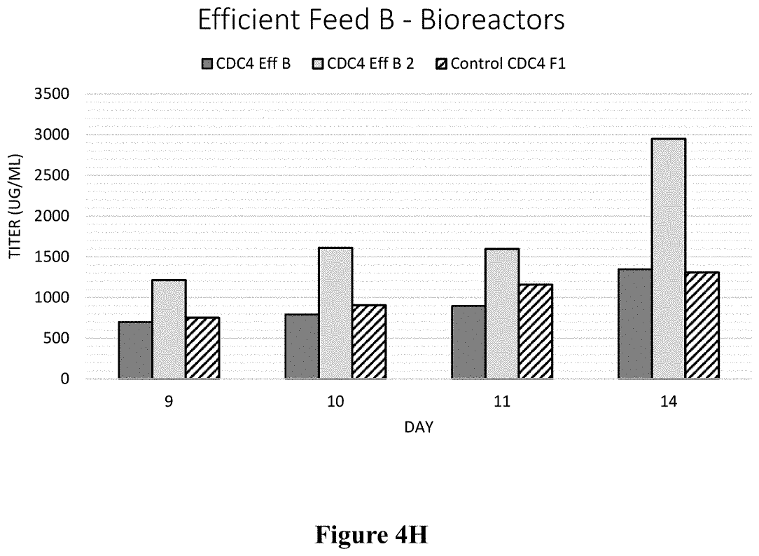

In total, there were six conditions. The first condition and the second condition was a standard strategy, in a bioreactor and in shake flasks (“CDC4 F1” and “SF CDC4 F1”, respectively). The third condition and the fourth condition was Efficient Feed B, fed every third day at 10% wv, in a bioreactor and shake flasks (“Eff B” and “SF Eff B”, respectively). The fifth condition and the sixth condition was Efficient Feed B 2 (a replicate run of Eff B), in a bioreactor and shake flasks (“Eff B 2” and “SF Eff B 2”, respectively). (FIG. 4A and Table 25)

TABLE 23

| | CDC4 F1 & | Eff B & | Eff B2 & |
|---|---|---|---|
| Condition | SF_CDC4 F1 | SF Eff B | SF Eff B2 |
| Feed Type | Feed 1 | Efficient Feed B | Efficient Feed B 2 |
| Volume | 1) 5%, 1.0x | 10 | 10 |
| (wv) % | 2) 7%, 1.0x | | |
| Schedule | 1) 3-8 | 3, 6, 9, 12 | 3, 6, 9, 12 |
| (days) | 2) 9-13 | | |

### Results & Discussion

The results of this experiment are shown in FIG. 4B-4G. In each graph, CDC4 F1 data is shown as squares with a solid line connection; SF CDC4 F1 data is shown as circles with a dashed line connection; Eff B data is shown as diamonds with a solid line connection; SF Eff B data is shown as triangles with a dashed line connection; Eff B 2 data is shown with “X” markers and a solid line connection; and SF Eff B 2 data is shown with “+” markers and a dashed line connection. FIG. 4B is a plot of viable cell density (VCD) vs. culture time for all six conditions. FIG. 4C is a plot of percent viability vs. culture time for all six conditions. FIG. 4D is a plot of glucose concentration (g/L) vs. culture time for the bioreactor conditions. FIG. 4E is a plot of VCD vs. culture time for the bioreactor conditions. FIG. 4F is a plot of lactate concentration (g/L) vs. culture time for the bioreactor conditions. FIG. 4G is a plot of ammonium concentration (mmol/L) vs. culture time for the bioreactor conditions. FIG. 4H is a bar plot of product titer produced in an Eff B bioreactor, an Eff B 2 bioreactor, and a control Feed 1 bioreactor over time.

In FIG. 4H the titer values (μg/mL) are as follows, Day 9: Eff B, Eff B 2, and CDC4 F1 are 699, 1214, and 752, respectively; Day 10: Eff B, Eff B 2, and CDC4 F1 are 792, 1611, and 907, respectively; Day 11: Eff B, Eff B 2, and CDC4 F1 are 897, 1596, and 1160, respectively; and Day 14: Eff B, Eff B 2, and CDC4 F1 are 1347, 2948, and 1308, respectively.

Table 26 shows the doubling time, IVCD, and Peak VCD for the Eff B, Eff B2, and CDC4 F1 conditions. Efficient Feed B with glucose corrections performed similarly to the control process. Efficient Feed B exhibits similar growth kinetics to BalanCD® CHO Feed 1. There were slightly improved IVCD and Peak VCD values on the second Eff B run.

TABLE 26

| Condition | Doubling time | IVCD | Peak VCD |
|---|---|---|---|
| CD C4 F1 | 35.73 | 93.08 | 12.29 |
| Eff B | 41.00 | 93.19 | 10.08 |
| Eff B 2 | 36.07 | 96.78 | 12.34 |

### Example 19—Product Characteristics

The evaluation of performance for various feeding strategies predominantly relied upon the analysis of growth characteristics, at least in part from the shake flasks data. Despite this important aspect, product titer and characteristics predominantly determine the effectiveness of an upstream platform. In this Example, titers and product quality data generated are presented for bioreactor runs (n=4), 2 control runs considered as baseline, and Feed 2 and Feed 4 experimental runs. Analysis was performed for samples on days 9, 10, 11, and 14.

After analysis of the product titer results, all samples fell within the target range of 386-789 mg/L. When comparing in-house control runs to experimental conditions, Feed 2 and Feed 4 had a greater titer at harvest (Table 27). When comparing VCD to titers among all conditions, little relation was observed. Within the present conditions, Feed 4 titer was the highest among evaluated conditions, resulting in a titer approximately >100 mg/L higher compared to the current control Feed 1 condition. Aside from titer, it is also considered to be beneficial to stay within the product quality limits to ensure successful lot release and similarity to the originator product.

TABLE 27
Overview of titer data generated from average baseline
and experimental Feed 2 and Feed 4 conditions in MOBIUS ® BRX
runs. Both experimental conditions produced a higher
harvest titer compared to baseline runs.

| | Feed 1 Ave. | Feed 2 | Feed 4 |
|---|---|---|---|
| Day | (mg/L) | (mg/L) | (mg/L) |
| 9 | 254.95 | 230.5 | 378.3 |
| 10 | 399.4 | 249.5 | 541.9 |
| 11 | 346.55 | 284.2 | 490.7 |
| 14 | 421 | 455.7 | 513.1 |

Product quality assessment was focused on the percentage of high mannose, afucosylation, and terminal galactose. In-house runs were compared to data generated from several satellite runs that were used to establish acceptance criteria for high mannose (8.1-17.1%), afucosylation (9-18%), and terminal galactose (11.6-27.5%). After analysis, all samples fell within acceptable release criterion, except for Feed 4, which had high mannose and low afucosylation (Table 28).

TABLE 28
Product quality characteristics (high mannose, afucose, terminal
galactose) for BRX runs compared to acceptable criteria
for standard batches. The Feed 4 BRX run was the only condition
to produce product out of specification (OOS).

| | High Mannose % | aFucose % | Terminal Galactose % |
|---|---|---|---|
| Standard 1 | 8.1-17.1 | 9.0-18.0 | 11.6-27.5 |
| Standard 2 | 8.29-10.68 | 9.54-11.96 | 17.19-18.35 |
| Feed 2 | 11.49 | 12.67 | 17.21 |
| Feed 4 | 3.28 | 5.98 | 25.71 |

According to a paper by Pacis et. al, the association of high osmolality with high mannose is not well understood; however, there is strong correlation between the two (Pacis E, Yu M, Autsen J, Bayer R, Li F. Effects of cell culture conditions on antibody N-linked glycosylation—what affects high mannose 5 glycoform. Biotechnol Bioeng. 2011; 108(10):2348-58). One hypothesis is that high OSMO media is negatively correlated to the product quality. An investigation of five CHO cell lines with high OSMO media (750 mOsm/kg) and low OSMO media (300 mOsm/kg) was carried out by Pacis et al. In all cell lines, Pacis's results showed increasing levels of high mannose during the length of the culture period, especially for high OSMO media. Other sources also state that increasing ammonia levels can inhibit intracellular pH enzymes due to the inhibition of glycosylation activities and results in increased levels high mannose glycoforms (Schneider M, Marison I W, von Stockar U. The importance of ammonia in mammalian cell culture. J Biotechnol. 1996 May 15; 46(3):161-85.)

Similar conclusions can be made about the correlation of lactate metabolism and pH with high OSMO media. Therefore, the contributing factors to the high OSMO levels are hypothesized to be generated from the accumulation of glucose, glutamine, ammonium, and lactate and may be the cause of the OOS product. OSMO buildup (>410 mOsm/kg) is associated with negative impact on the growth profile of CHO cells (Xu S, Hoshan L, Chen H. Improving lactate metabolism in an intensified CHO culture process: productivity and product quality considerations. Bioprocess Biosyst Eng. 2016 Nov. 1; 39(11):1689-702).

### Medium Adaptation Studies

### Example 20—EX-CELL® Advanced Medium Adaptation

Medium adaptation studies were explored to increase cell growth characteristics and product quality and yield. EX-CELL® Advanced CHO Fed-batch Medium was the first media to be studied for adaptation. This media is chemically-defined and contains no animal derived components. The manufactures indicated that this media both supported growth and productivity across a diverse set of CHO cell lines in fed-batch cultures, while still allowing for the flexibility of adjusting protein quality attributes.

### Methodology/Experimental Design

In all adaptation studies performed on the DG44 cell line, the determining factor of when the cells are considered adapted is based on doubling times. When three consecutive consistent doubling times are produced, the cell line is considered adapted to the new media. A method of direct adaptation was employed for these studies.

For the EX-CELL® adaptation, the following strategy was employed. Starting from a CD-C4 media culture, five 30 mL non-baffled working volume flasks were inoculated at a seeding density of 0.5 VCD in EX-CELL® media (supplemented with 6 mmol of glutamine). Three of these flasks were cultured in batch until death occurred to study the growth characteristics. Two of the flasks were pooled on day 3, used to passage the next five flasks with EX-CELL® media and freeze down cryovials (1 mL 1×10⁷). This pattern of passaging was repeated until three consecutive doubling times were observed.

### Results & Discussion

The EX-CELL® adaptation study was concluded after 13 passages with an average doubling time of ˜28 hours. A relatively low doubling time exhibited by passage 4 was suspected to be caused by sampling error.

Comparing the results from both CD-C4 and EX-CELL® cultures grown in batch, EX-CELL® improved on both peak VCD and IVCD values, in addition to comparable doubling times (Table 29).

Cell concentrations above 7 million were achieved usually around day 7, in addition to high viability being maintained till around day 8. Unfortunately, no reliable glutamine results were obtained; this is hypothesized to be an instrument error. Based on metabolite consumption, there was a limitation in available carbon source starting day 7; glucose levels steadily decreased during the culture and lactate was also consumed.

TABLE 29
Peak VCD, IVCD, and Doubling Times in CD-C4 (n =
3), and EX-CELL ® Advanced Medium (n=3) in
batch mode. A slightly higher peak VCD and IVCD was observed
in EX-CELL ® adapted cell lines, despite similar doubling times.

| Condition | CD-C4 | EX-CELL |
|---|---|---|
| Peak VCD (×10⁶ | 6.64 | 7.16 |
| cells/mL) | | |
| IVCD | 29.47 | 40.97 |
| (×10⁶ cells × hr/mL) | | |
| Doubling Time | ~28 | ~28 |
| (hours) | | |

### Example 21—EX-CELL® Fed-Batch

Due to the improvement on doubling time and IVCD for EX-CELL® adapted cells in Example 20, a fed-batch experiment was completed. EX-CELL® Advanced CHO Feed 1 was developed in conjunction with EX-CELL® Fed-Batch medium and therefore was chosen to feed the new adaptation.

### Methodology/Experimental Design

Three feeding strategies were chosen for this experiment. The first two conditions were done in EX-CELL® Fed-Batch medium with EX-CELL® adapted cells. One set of shake flasks were fed with Advanced CHO Feed 1 (“SF ACF1”) following the vendor's recommendation, 8% working volume starting day 3, then fed on every odd day. Next, EX-CELL® adapted cells were fed following a standard feeding strategy using BalanCD® CHO Feed 1 (“SF BF1”). Lastly, a control using a standard feeding strategy with CDC4 (“SF CDC4 F1”) adapted adalimuamb cell line were used as a control.

TABLE 30
Experimental conditions evaluating different feeding strategies
using EX-CELL ® adapted cell lines.

| Condition | SF_ACF1 | SF_BF1 | SF_CDC4 F1 |
|---|---|---|---|
| Feed Type | Advanced CHO | BalanCD ® CHO | BalanCD ® |
| | Feed 1 | Feed 1 | CHO Feed 1 |
| Volume (wv) % | 8% | 1) 5% | 1) 5% |
| | | 2) 7% | 2) 7% |
| Schedule (days) | Starting day 3, | 1) 5% | 1) 5% |
| | then every odd | 2) 7% | 2) 7% |
| | day | | |

### Results & Discussion

Selected results are shown in Table 31. Table 31 shows the doubling time and IVCD for the three experimental conditions. The lowest doubling time and the highest IVCD was seen in the EX-CELL® Advanced CHO Feed 1 (SF ACF1) condition compared to EX-CELL® BalanCD® CHO Feed 1 (SF BF1) and CDC4 BalanCD® CHO Feed 1 (SF CDC4 F1). The peak VCD for the 3 conditions was achieved by SF ACF1 at 5.78×10⁶ cells. Advanced CHO Feed 1 (SF ACF1) reached a higher VCD compared to the control (SF CDC4 F1) and BalanCD® CHO Feed 1 (SF BF1) reached the lowest VCD. Percent Viability between the 3 conditions did not represent any observable differences.

Glucose levels for the control (SF CDC4) and ACF1 (SF ACF1) conditions did not affect VCD, but when comparing BF1 (SF BF1) to the control, a higher glucose level and lower VCD was observed, indicating the glucose was not a limiting factor. Lactate levels deviated for the EX-CELL® conditions (SF ACF1 and SF BF1) starting day 8, as the relative lactate levels dropped compared to the control. The lactate difference did not appear to affect VCD, indicating that lactate is likely not an inhibitory factor. Glutamine levels for the EX-CELL® adapted cells (SF ACF1 and SF BF1) started and remained higher for days 0-7, and then were similar till the end of the run compared to the control. There is some data missing for days 7-9 due to equipment malfunction. Each condition deviated in glutamine concentration starting day 3. Although ACF1 and BF1 had higher ammonium through the course of the run compared to the control, no impact on VCD was observed. The pH and osmolality parameters did not have an observable effect on VCD.

ACF1, when compared to the standard feeding strategy, had a higher peak VCD and IVCD over the 14-day run as well as the lowest doubling time. The data from this experiment may provide a baseline for EX-CELL® adapted cells.

TABLE 31
Highest VCD, IVCD and DT for the EX-CELL ® adapted
experimental conditions compared to the control conditions.
EX-CELL ® adapted cells utilizing advanced CHO
Feed 1 outperformed the control and other experimental conditions.

| | SF_ACF1 | SF_BF1 | SF_CDC4F1 |
|---|---|---|---|
| Highest VCD | 5.74 | 5.49 | 5.08 |
| (10⁶ cells/mL) | | | |
| IVCD (10⁶ cell-hrs/mL) | 55.07 | 51.22 | 43.36 |
| DT(Hours) | 27.59 | 30.14 | 31.09 |

### Example 22—HyClone™ ActiPro™

An initial adaptation study was performed using HyClone™ ActiPro™ media by GE Healthcare Life Sciences to investigate changes to cell growth characteristics and product quality and yield. HyClone™ ActiPro™ media is chemically defined, animal-derived component-free (ADCF) and hydrolysate/peptide free. According to the manufacturer, the media is also optimized for high yield protein production in CHO batch and fed-batch processes.

### Methodology/Experimental Design

A direct adaptation was done with 30 mL working volume non-baffled shake flasks at a seeding density of (0.5×10⁶ cells/mL) from cells cultured in CD-C4. These batch cultures were incubated at 37° C. and 5% CO₂. On day three, two flasks with the highest viability were pooled and sub-cultured into 5 shake flasks following the same inoculation parameters. This procedure was maintained until three consistent doubling times are observed in three consecutive passages and thus, the cells were considered to be adapted. Growth curve data, doubling times and metabolites were collected and analyzed in comparison to the control of CD-C4 media grown cells. A frozen cell bank was established once adaptation was complete.

With the adapted cells, a batch study was done to characterize HyClone™ ActiPro™ cell growth compared to CD-C4 and see the effects of added Glutamine on cell growth. Two experimental variables were studied alongside a control CD-C4 batch growth. Three sets of triplicate non-baffled 30 mL shake flasks were inoculated at a seeding density of (0.5×10⁶ cells/mL) and cultured in: HyClone™ ActiPro™ with no added Glutamine (“HYC w/o Gln”), HyClone™ ActiPro™ with 3 mmol added Glutamine (“HYC w/Gln”), and CD-C4 media (“CD-C4”).

### Results and Discussion

In analyzing growth kinetics, cells cultured in HyClone™ w/o Gln showed the lowest overall peak viable cell density VCD (4.04×10⁶ cells/mL), and the cells cultured in CD-C4 showed the highest VCD (6.64×10⁶ cells/mL). HyClone™ w/Gln showed similar peak VCD values to CD-C4 at (6.31×10⁶ cells/mL). CD-C4 saw the lowest doubling time (27.8 hours), followed by HyClone™ w/Gln (29.6 hours), and lastly HyClone™ w/o Gln (36.6 hours) (Table 32). Integral viable cell density was the highest for HyClone™ w/Gln at 33.56 (×10⁶ cells-hours/mL), followed by CD-C4 at 29.47 (×10⁶ cells-hours/mL), and lowest for HyClone™ w/o Gln at 22.80 (×10⁶ cells-hours/mL) (Table 32). When comparing HyClone™ with CD-C4, a sustained stationary phase, as well as a higher percent viability, was observed in the HyClone™ w/Gln through the course of the experiment.

From days 1-5, HyClone™ w/Gln and CD-C4 exhibited similar glutamine consumption rates; after day 5, CD-C4 culture glutamine levels increased. The variation in glutamine levels is consistent with the composition of media and the differences in VCD. No significant difference was seen in glucose consumption.

Examining the metabolite levels, CD-C4 showed the highest peak lactate production (2.39 g/L). The HyClone™ experimental groups exhibited similar peak lactate production levels at around (1 g/L). Ammonium production was relatively similar for HyClone™ w/Gln and CD-C4—with levels peaking around 6.0 mmol/L, while and HyClone™ w/o Gln peaked at about 3.0 mmol/L. No significant differences were observed in Osmolality or pH.

The input levels of glucose and glutamine were compared to lactate levels to determine which input was more responsible for metabolite generation. A relatively higher lactate level was observed in CD-C4, while the two HyClone™ conditions were similar to each other. As there was no distinct variation in glucose levels, glutamine was concluded to be more responsible for metabolite generation.

In comparison of the three conditions, the highest VCD was observed in CD-C4, with HyClone™ w/Gln showing comparable peak VCD. HyClone™ w/Gln saw the highest IVCD as a result of the sustained stationary phase and relatively higher percent viability through the course of the experiment combined with a lower lactate level.

TABLE 32
HyClone ™ Adaptation Study and CD-C4 Peak
VCD, IVCD, and Doubling Time. HyClone ™ containing
glutamine outperformed HyClone ™ without
glutamine and control cell line in batch mode.

| Condition | HYC w/o Gln | HYC w/Gln | CD-C4 |
|---|---|---|---|
| Peak VCD (×10⁶ | 4.04 | 6.31 | 6.64 |
| cells/mL) | | | |
| IVCD | 22.80 | 33.56 | 29.47 |
| (×10⁶ | | | |
| cells × hr/mL) | | | |
| Doubling Time | 36.6 | 29.6 | 27.8 |
| (hours) | | | |

### Example 23—Feed Concentration Evaluation

A study was conducted to analyze and investigate the performance of cells with a different concentrations of Feed 4. Evaluations were conducted in bioreactors.

### Methodology/Experimental Design

The first condition was the control using BalanCD® CHO Feed 1 with a standard strategy (“CHO F1”). The second condition was 0.8× concentration of BalanCD® CHO Feed 4 (“0.8× F4”). The third condition was 1× concentration of BalanCD® CHO Feed 4 fed every day (“1× F4”). For all conditions, CDC4 was used as the base culture medium (Table 33).

TABLE 33

| Condition | CHO_F1 | 0.8X F4 | 1X_F4 |
|---|---|---|---|
| Feed Type | Feed 1 | Feed 4 0.8X | Feed 4 1X |
| Volume | 4% | 4% | 4% |
| (wv) % | | | |
| Schedule | 1) 3-7 | 3, 5, 7, | 3, 5, 7, |
| (days) | 2) 8-13 | 9 11, 13 | 9 11, 13 |

### Results & Discussion

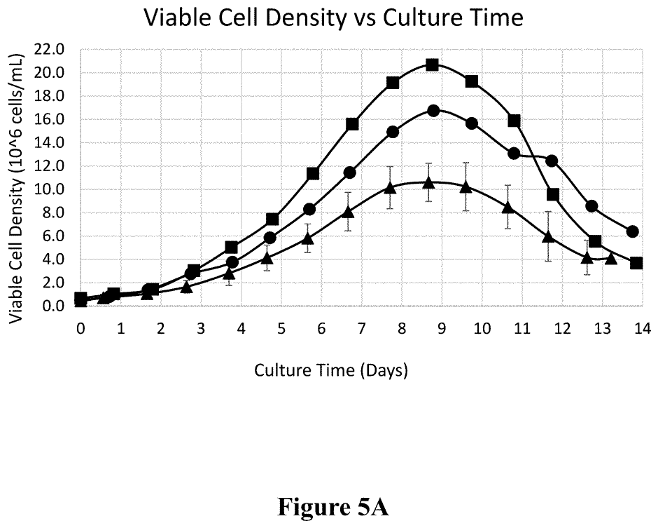

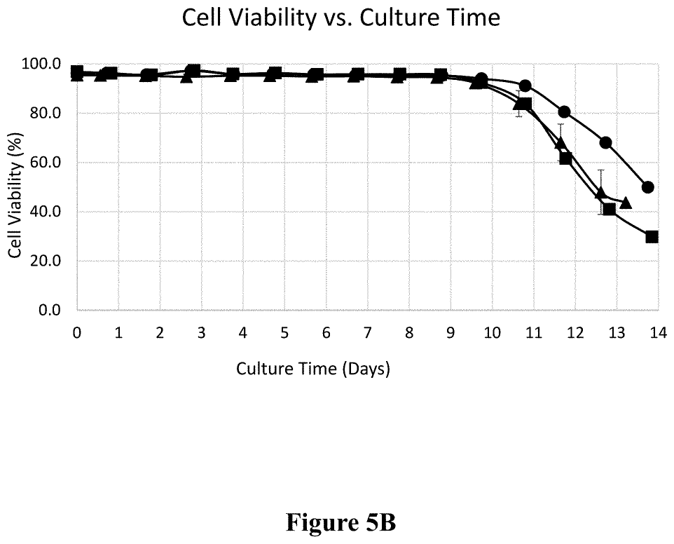

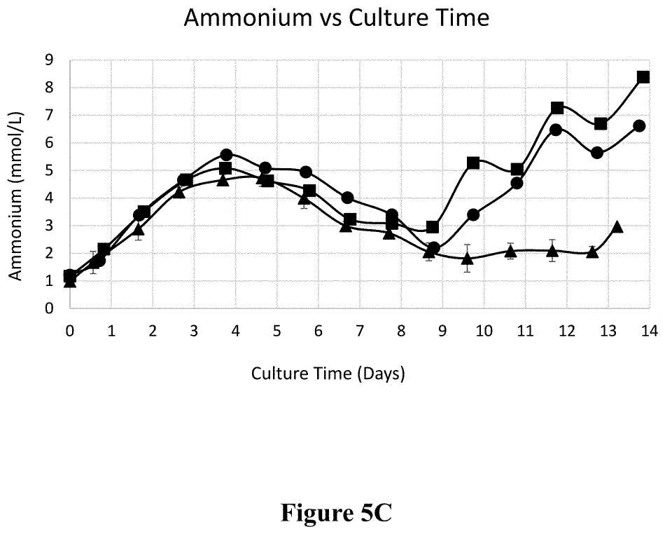

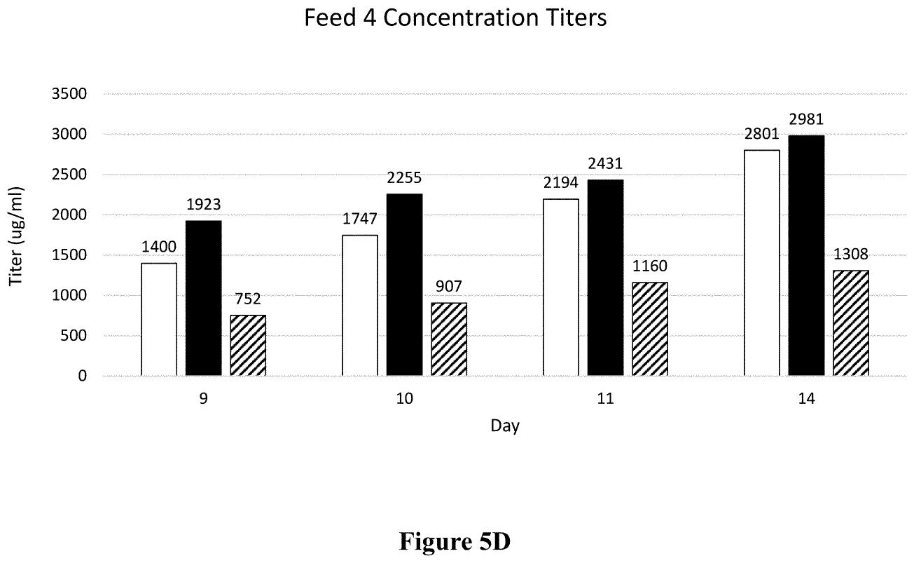

The results of this experiment are shown in FIG. 5A-5D. In FIGS. 5A-5C, CDC4 F1 data is shown as triangles with a solid line connection; 0.8× F4 data is shown as circles with a solid line connection; 1× F4 data is shown as squares with a solid line connection. FIG. 5A is a plot of viable cell density (VCD) vs. culture time. FIG. 5B is a plot of percent viability vs. culture time. FIG. 5C is a plot of ammonium concentration (mmol/L) vs. culture time. In the experimental conditions (0.8× F4 and 1× F4), substantial improvements were seen in peak cell density.

FIG. 5D is a bar plot of product titer for 0.8× F4 (white), 1× F4 (black), and control CDC4 F1 (striped) cultures over time. Cell viability was higher at days 10-14 for 0.8× F4. Ammonium levels began to rise on the same day that peak cell densities were reached. In FIG. 5D the titer values (μg/mL) are listed on the plot. In the experimental conditions (0.8× F4 and 1× F4), a 2-3-fold increase in titer was observed.

### Example 24—Culture Medium and Adaptation Evaluation

A study was conducted to investigate the effects of medium adaptation and medium switch for various culture media. Evaluations were conducted in bioreactors.

### Methodology/Experimental Design

In total, there were 6 conditions. Table 34 below lists the media, feed, and strategy for each condition.

TABLE 34

| | | Adapt or | |
|---|---|---|---|
| ID | Medium | Switch | Feed Strategy |
| M2A-CF | ExCell | Adapt | Feed 1, Control Strategy |
| M3A-CF | lactate-enriched | Adapt | Feed 1, Control Strategy |
| | CD-C4 | | |
| M1S-CF | Hyclone ActiPro | Switch | Feed 1, Control Strategy |
| M1S-NF | Hyclone ActiPro | Switch | Cell Boost 7a |
| | | | (2%)/7b(0.4%) |
| M1A-CF | Hyclone ActiPro | Adapt | Feed 1, Control Strategy |
| MC-CF | CD-C4 | CNTRL | Feed 1, Control Strategy |

The lacate enriched CDC4 medium contained 30 mM sodium lactate. The feed strategy for M1S-NF was Cell Boost 7a and 7b, feed with 2% of 7a and 0.4% of 7b from day 3 to day 13.

### Results & Discussion

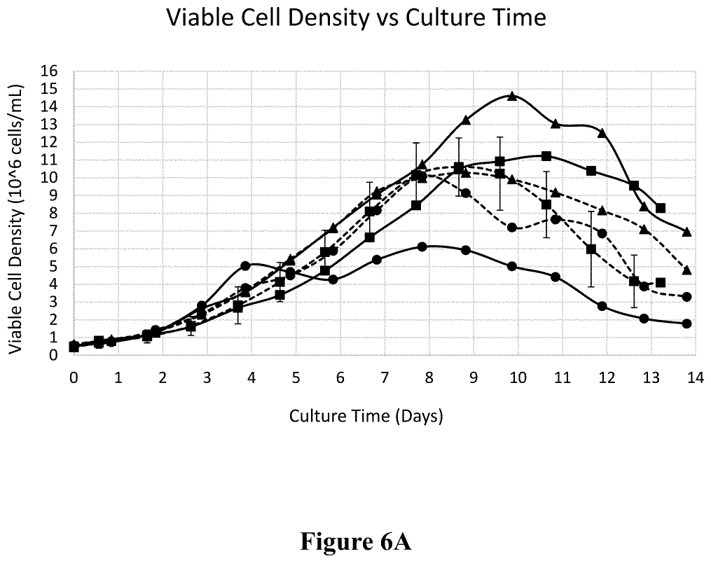

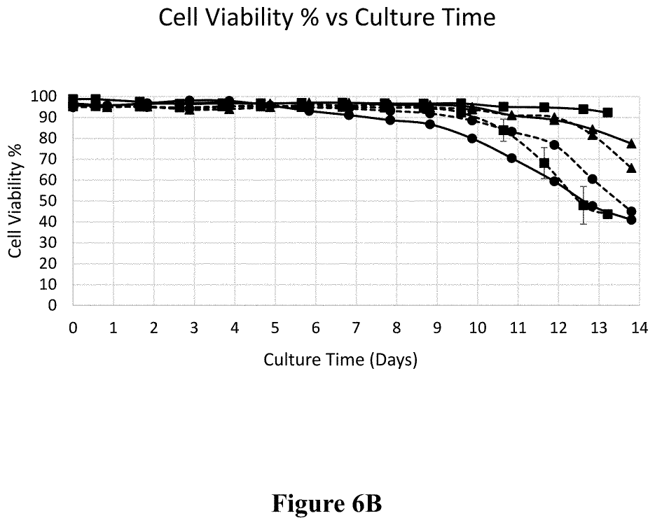

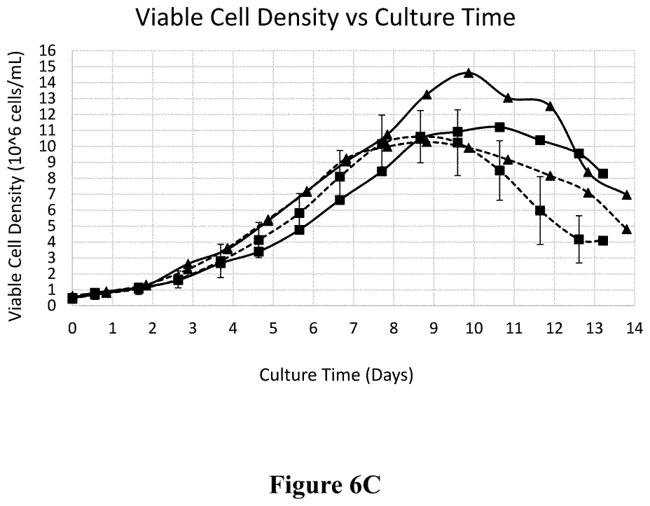

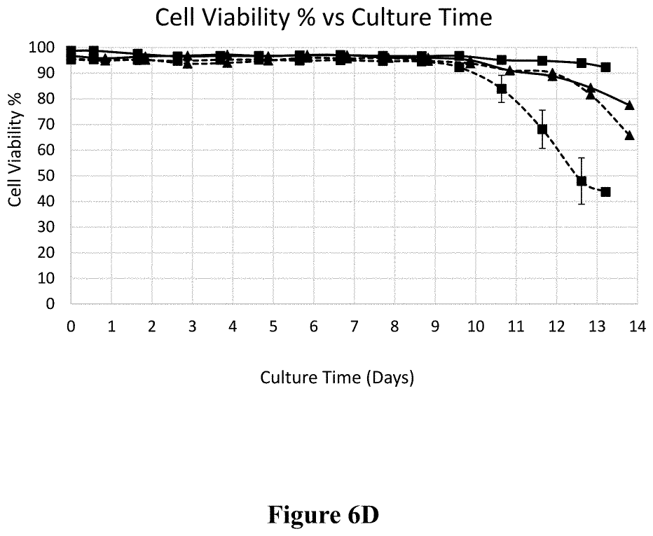

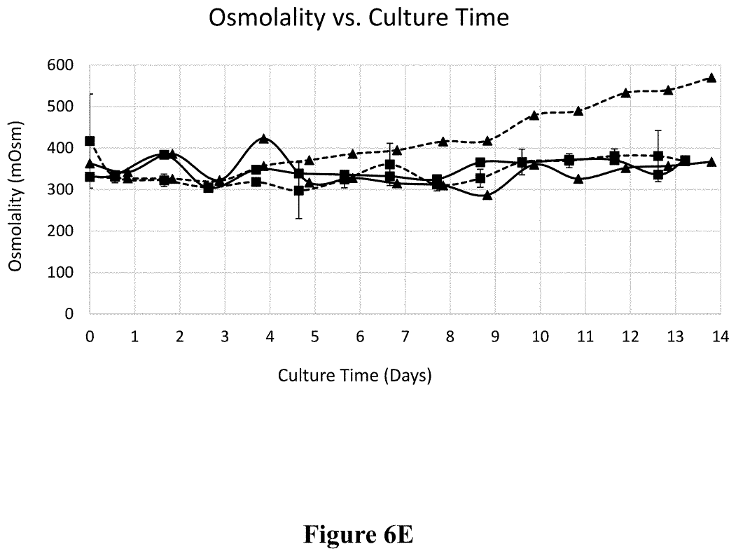

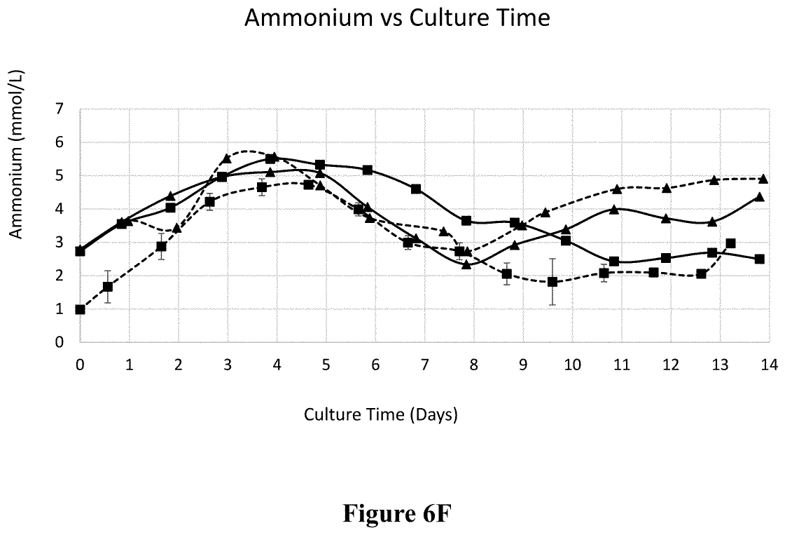

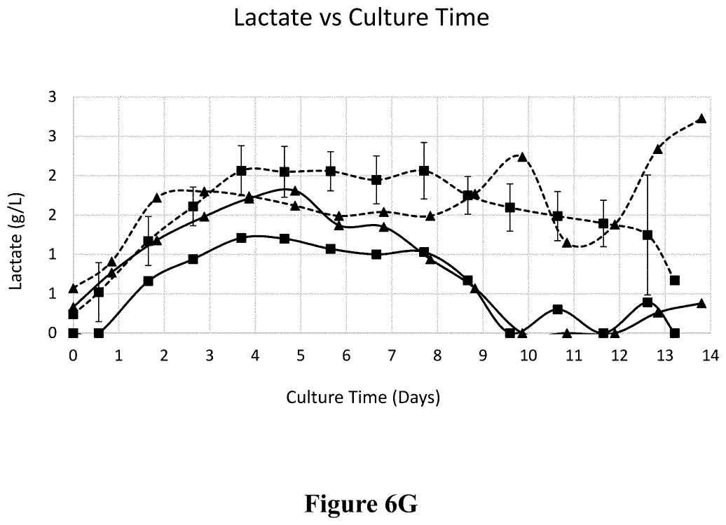

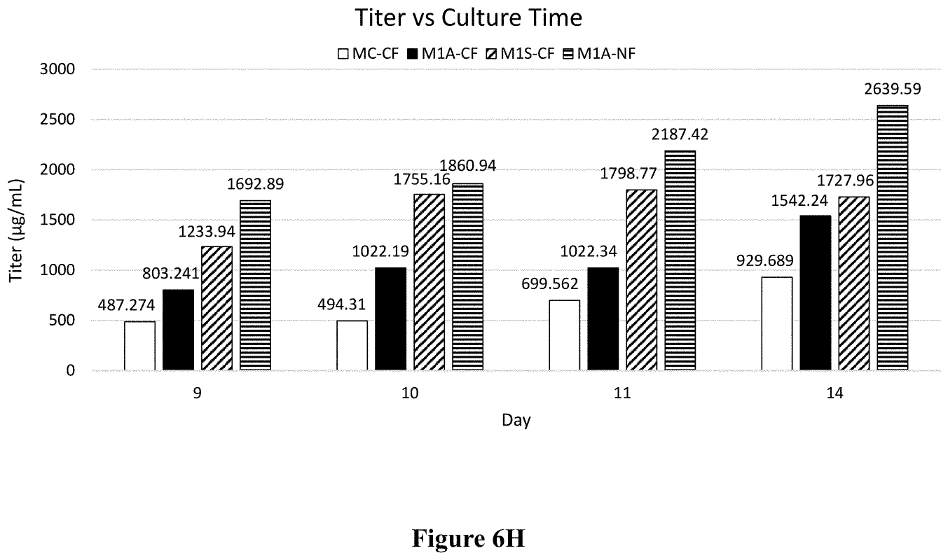

The results of this experiment are shown in FIG. 6A-6H. In FIGS. 6A-6G, M2A-CF data is shown as circles with a solid line connection; M3A-CF data is shown as circles with a dashed line connection; M1S-CF data is shown as triangles with a solid line connection; M1S-NF data is shown as triangles with a dashed line connection; M1A-CF data is shown as squares with a solid line connection; and MC-CF data is shown as squares with a dashed line connection.

FIG. 6A is a plot of viable cell density (VCD) vs. culture time for all conditions. FIG. 6B is a plot of percent viability vs. culture time for all conditions. Cell cultures with Hyclone ActiPro medium outperformed other cultures with different media. Specifically, cell cultures with Hyclone ActiPro medium had higher VCD and higher cell viability at termination of the culture run.

FIG. 6C is a plot of viable cell density (VCD) vs. culture time for conditions with Hyclone ActiPro or CD-C4 control media. FIG. 6D is a plot of percent viability vs. culture time for conditions with Hyclone ActiPro or CD-C4 control media. FIG. 6E is a plot of osmolality (mOsm) vs. culture time for conditions with Hyclone ActiPro or CD-C4 control media. FIG. 6F is a plot of ammonium concentration (mmol/L) vs. culture time for conditions with Hyclone ActiPro or CD-C4 control media. FIG. 6G is a plot of lactate concentration (g/L) vs. culture time for conditions with Hyclone ActiPro or CD-C4 control media. Cell cultures with Hyclone ActiPro medium sustained logner stationary phases. M1A-CF displayed strong growth characteristics. Ammonium and lactate accumulation contributed to increased osmolality in M1S-NF culture.

FIG. 6H is a bar plot of product titers over time in for conditions with Hyclone ActiPro or CD-C4 control media. MC-CF data is shown as white bars; M1A-CF data is shown as black bars; M1S-CF data is shown as diagonal-striped bars; and M1S-NF data is shown as horizontal-striped bars. In FIG. 6H, the titer values (μg/mL) are displayed on the figure. All conditions with Hyclone ActiPro had substantially higher titers than the condition with the control CDC4 medium. Surprisingly, higher titers were observed in conditions where cells were not adapted to the culture medium prior to being added to the bioreactor (M1S-CF, M1S-NF). Interestingly, despite less favorable growth kinetics, M1S-NF displayed 2-3 fold improvement in titer.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of producing a recombinant protein, the method comprising:
(a) providing a cell culture comprising a CHO cell in a liquid culture medium, wherein the CHO cell comprises a nucleic acid encoding a recombinant protein, wherein the cell culture has a volume;
(b) fed-batch culturing the cell culture of step (a) under conditions sufficient for the CHO cell to produce the recombinant protein, wherein:
the fed-batch culturing comprises adding a volume of a first feed culture medium comprising 0.8× to 1.0× BalanCD™ CHO Feed 4 on about day 2 to about day 5 of the culture and adding a volume of a second feed culture medium comprising 0.9× to 1.1× BalanCD™ CHO Feed 2 on about day 6 to about day 13; and
(c) recovering the recombinant protein from the CHO cell or the liquid culture medium.

2. The method of claim 1, wherein one or more of the liquid culture medium, the first feed culture medium, and the second feed culture medium further comprise(s) a concentration of N-acetylglucosamine sufficient to maintain a concentration of N-acetylglucosamine in the cell culture of about 2 mM to about 8 mM relative to the volume of the cell culture in step (a).

3. The method of claim 1, wherein the fed-batch culturing further comprises adding a volume of a supplement comprising a concentration of N-acetylglucosamine sufficient to maintain a concentration of N-acetylglucosamine in the cell culture of about 2 mM to about 8 mM.

4. The method of any one of claims 1-3, wherein the volume of the first feed culture medium added on about day 2 to about day 5 is about 4% to about 10% of the volume of the cell culture in step (a) per day.

5. The method of claim 4, wherein the volume of the first feed culture medium added on about day 2 to about day 5 is about 5% of the volume of the cell culture in step (a) per day.

6. The method of any one of claims 1-5, wherein the volume of the second feed culture medium added on about day 6 to about day 13 is about 4% to about 10% of the volume of the cell culture in step (a) per day.

7. The method of claim 6, wherein the volume of the second feed culture medium added on about day 6 to about day 13 is about 5% to about 7% of the volume of the cell culture in step (a) per day.

8. The method of any one of claims 1-7, wherein the first feed culture medium comprises about 0.8× BalanCD CHO Feed 4.

9. The method of any one of claims 1-8, wherein the second feed culture medium comprises about 1.0× BalanCD™ CHO Feed 2.

10. The method of any one of claims 1-9, wherein the fed-batch culturing further comprises adjusting the temperature of the culture on about day 7 to about day 8.

11. The method of claim 10, wherein the temperature of the culture is adjusted from a first temperature of about 35-38° C. to a second temperature of about 28-34.9° C.

12. The method of claim 10, wherein the temperature of the culture is adjusted from a first temperature of about 36.5° C. to a second temperature of about 34° C.

13. The method of any one of claims 1-12, wherein the fed-batch culturing further comprises maintaining the pH of the cell culture at about 6.7 to about 7.1.

14. The method of claim 13, wherein upon the cell culture obtains a pH of 6.9, the pH is maintained at about 6.88 to about 6.92.

15. The method of any one of claims 1-14, wherein the fed-batch culturing further comprises maintaining the dO₂ of 40%.

16. The method of any one of claims 1-15, wherein the fed-batch culturing further comprises agitating the cell culture at about 10 RPM to about 500 RPM.

17. The method of claim 16, wherein the fed-batch culturing further comprises agitating the cell culture at about 180 RPM to about 220 RPM.

18. The method of any one of claims 1-15, wherein the fed-batch culturing further comprises agitating the cell culture using an impeller tip speed of 0.4 m/s to about 4.0 m/s.

19. The method of any one of claims 1-15, wherein the fed-batch culturing further comprises agitating the cell culture using an impeller power consumption per volume of about 10 W/m³ to about 35 W/m³.

20. The method of any one of any one of claims 1-19, wherein the recovering in step (c) occurs on day 14.

21. The method of any one of claims 1-19, wherein the cell culture has a percent of cell viability and wherein the recovering in step (c) occurs when the percent of cell viability falls below a value selected from the group consisting of about 70%, about 60%, about 50%, about 40%, and about 30%.

22. The method of any one of claims 1-21, wherein the CHO cell is a DG44 cell.

23. The method of any one of claims 1-22, wherein the first feed culture medium and the second feed culture medium further comprises about 4 g/L glucose to about 6 g/L glucose.

24. The method of claim 23, wherein the first feed culture medium and the second feed culture medium comprises about 5 g/L glucose.

25. The method of any one of claims 1-24, wherein the recombinant protein is a fusion protein, antibody, or antibody fragment.

26. The method of any one of claims 1-25, further comprising:
generating the cell culture of step (a) comprising inoculating the liquid culture medium with a population of the CHO cells.

27. The method of claim 26, wherein the population of the CHO cells has not been previously cultured in the liquid culture medium.

28. The method of any one of claims 1-27, wherein the liquid culture medium is HyClone™ ActiPro™.

29. The method of any one of claims 1-27, wherein the liquid culture medium is CD-C4.

30. The method of any one of claims 1-29, further comprising:
purifying the recovered recombinant protein.

31. The method of claim 30, further comprising:
formulating the purified recombinant protein into a pharmaceutical composition.

32. A recombinant protein produced by the method of any one of claims 1-31.

33. A pharmaceutical composition produced by the method of claim 31.

34. A method of treating a subject in need thereof comprising administering to the subject a therapeutically effective amount of the recombinant protein of claim 32 or the pharmaceutical composition of claim 33.


SEQUENCE LISTING (2 sequences)

SEQ ID NO: 1 - protein, 110 aa, Artificial
  Ranibizumab Light Chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 2 - protein, 158 aa, Artificial
  Ranibizumab Heavy Chain
SGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES